(19) United States
(12) Patent Application Publication
(10) Pub. No.: US 2024/0325562 A1
(43) Pub. Date: Oct. 3, 2024

(54) INTERNALIZING BINDING MOLECULES

(71) Applicant: LinXis B.V., Amsterdam (NL)

(72) Inventors: Bart Antonius NIJMEIJER, Amsterdam (NL); Paulus Martinus Petrus VAN BERGEN EN HENEGOUWEN, Amsterdam (NL); Niels Jurriaan SIJBRANDI, Amsterdam (NL); Eugen MERKUL, Amsterdam (NL); Joey Armand MUNS, Amsterdam (NL); Jan Hendrik SCHOOTEN, Amsterdam (NL); Sebas Daniel PRONK, Amsterdam (NL)

(73) Assignee: LinXis B.V., Amsterdam (NL)

(21) Appl. No.: 18/245,066

(22) PCT Filed: Sep. 15, 2021
(86) PCT No.: PCT/NL2021/050559

§ 371 Date: Mar. 13, 2023

(30) Foreign Application Priority Data
Sep. 16, 2020 (NL) 2026478

(51) Int. Cl. A61K 47/68; A61K 49/00

(52) U.S. Cl. CPC A61K 47/6849; A61K 47/6843; A61K 49/0002

(57) ABSTRACT

The invention relates to the field of binding molecules comprising at least one single variable antibody domain, targeted at receptors present on fibrogenic effector cells, such as activated hepatic stellate cells in liver fibrosis, activated interstitial fibroblasts or vascular smooth muscle cells in lung fibrosis, activated mesangial- or interstitial fibroblasts in renal fibrosis, activated dermal fibroblasts in systemic sclerosis/scleroderma, activated fibroblasts in inflammatory bowel disease, or tumor-supporting stromal fibroblasts in sclerosing malignant tumors. More in particular, the invention relates to internalizing binding molecules for cell-specific targeted delivery of anti-fibrotic substances. The invention also relates to a binding molecule comprising at least two single variable antibody domains, each targeting a receptor on said fibrogenic effector cells. The invention further relates to nucleic acids encoding such binding molecules, a host cell for expression of such binding molecules and to methods for preparing such binding molecules. The invention further relates to pharmaceutical compositions that comprise such binding molecule and to uses of such binding molecules and/or compositions, in particular for prophylactic, therapeutic or diagnostic purposes.

### TECHNOLOGICAL FIELD

The invention relates to the field of binding molecules comprising at least two single variable antibody domains, targeted at receptors present on fibrogenic effector cells, which includes activated myo-fibroblasts and similar and/or related pro-fibrotic cells and/or contractile cells. More in particular, the invention relates to internalizing binding molecules for cell-specific targeted delivery of anti-fibrotic substances. The invention also relates to a binding molecule comprising at least two single variable antibody domains, each targeting a receptor that is selectively (over-) expressed on fibrogenic effector cells, which comprise the activated, contractile, or pro-fibrotic myo-fibroblast-like cells that derive from activated hepatic stellate cells in liver fibrosis, activated alveolar fibroblasts or -smooth muscle cells in lung fibrosis, activated mesangial or interstitial fibroblasts in kidney fibrosis, activated dermal fibroblasts in systemic sclerosis/scleroderma, activated intestinal fibroblasts in chronic bowel inflammation, or activated tumor stromal fibroblasts. The invention further relates to nucleic acids encoding such binding molecules; a host cell for expression of such binding molecules and to methods for preparing such binding molecules. The invention further relates to pharmaceutical compositions that comprise such binding molecules and to uses of such binding molecules and/or pharmaceutical compositions, in particular for prophylactic, therapeutic or diagnostic purposes.

### BACKGROUND OF THE INVENTION

Organ fibrosis is a leading cause of morbidity and mortality. Fibrosis is the result of an overshoot of the natural response of the body to ‘heal’ in response to injury. Key events in fibrosis include the emergence of fibrogenic effector cells. Depending on the affected tissue, such fibrogenic effector cells may stem from different types of resident mesenchymal progenitors. After injury, these progenitor cells may become activated through mediators such as transforming growth factor beta (TGFB) or the platelet-derived growth factors (PDGFs), to transdifferentiate into a more general myo-fibroblastic phenotype. These contractile, myo-fibroblast-like cells secrete extracellular matrix (ECM-) proteins and pro-fibrotic mediators and are the key effector cells in fibrosis, causing replacement of functional normal tissue with non-functional scar tissue.

Fibrosis may theoretically affect any organ. Prominent clinical manifestations of fibrosis include fibrosis of liver, kidney, lung, skin, and gut. Liver fibrosis generally occurs secondary to Non-Alcoholic Liver Disease (NALD), Alcoholic Liver Disease (ALD), biliary obstruction, or viral hepatitis, and generally progresses to an end stage known as cirrhosis. Renal fibrosis may be idiopathic, e.g. IgA nephropathy (IGAN), or secondary to e.g. Diabetes or Hypertension. Pulmonary fibrosis may be idiopathic or secondary to e.g. chronic obstructive pulmonary disease (COPD) or systemic sclerosis (SS). Fibrosis of the gut may be secondary to (chronic) inflammatory bowel disease (IBD), e.g. Crohn's disease (CD) or Colitis Ulcerosa (CU). Remarkably, even certain malignant tumors, known as sclerosing tumors, comprise a fibrotic component on which their growth may at least partially depend.

The primary consequence of fibrosis is the destruction of the natural tissue architecture and, consequently, loss of organ function. Fibrosis of liver or kidneys affects the normal function of these organs in detoxification and homeostasis, fibrosis of the lung affects oxygenation, and fibrosis of the gut affects digestive processes. Secondary consequences of fibrosis may arise from the increased resistance of the fibrotic vasculature, e.g. portal hypertension in liver cirrhosis and pulmonary hypertension in lung fibrosis. These secondary pathologies may spark acute life-threatening events, such as oesophageal bleeding in portal hypertension, or cardiac failure in pulmonary hypertension.

Because of its profound consequences, fibrosis is an extremely debilitating condition that greatly impacts Quality of Life (QoL). Treatment options for fibrosis are limited. Early stage secondary fibrosis may be prevented or ameliorated by treating the primary affliction, e.g. by treating the underlying injuries (e.g. by modulating lipid metabolism in ALD, or through antiviral therapy in viral hepatitis), or by treating the underlying inflammatory processes (e.g. by anti-inflammatory pharmacotherapy in inflammatory bowel disease). For established fibrosis however, no effective treatment exists, and in end-stage fibrosis transplantation remains the only life-saving intervention.

Advances in the understanding of the pathogenesis of liver fibrosis, and advances in the understanding of the molecular biology of the putative fibrogenic effector cells, have sparked the development of novel therapeutics. These (often small) molecules such as Rho-kinase inhibitors and JAK-2 kinase inhibitors has overall however been hampered by their cumbersome safety profile. Thus, the need for safe and effective therapeutics that target the heart of the fibrotic process remains large and unmet.

### SUMMARY OF THE INVENTION

The present invention provides for an improved therapeutic approach against medical conditions in which transdifferentiated mesenchymal cells play a central effector role. Based on scientific consensus, these medical conditions entail most clinically recognized forms of fibrosis including fibrosis of the liver, lung or kidney, systemic sclerosis, inflammatory bowel disease and even certain (sclerosing) types of tumors such as some cases of breast cancer, colon cancer, lung cancer or prostate cancer. The invention entails a binding molecule comprising at least one single variable antibody domain and at least one diagnostic and/or therapeutic molecule, or comprising at least two, and preferably two, single variable antibody domains and at least one diagnostic and/or therapeutic molecule, wherein at least one of the single variable antibody domains, preferably two single variable antibody domains, is/are able to specifically bind to a transmembrane receptor that is expressed on fibrogenic effector cells, such as for example activated hepatic stellate cells in liver fibrosis, activated alveolar fibroblasts or -smooth muscle cells in lung fibrosis, activated mesangial or interstitial fibroblasts in kidney fibrosis, activated dermal fibroblasts in systemic sclerosis/scleroderma, activated intestinal fibroblasts in chronic bowel inflammation, or even activated tumor stromal fibroblasts.

Such binding molecule enables targeting of such fibrogenic effector cells, preferably through binding to a platelet derived growth factor receptor-beta (PDGFRB; also referred to as platelet derived growth factor receptor beta and also abbreviated as PDGFBR PDGFRβ) or to an insulin-like growth factor 2 receptor (IGF2R), which are both highly (i.e. ‘over-’) expressed on such cells as compared to the progenitor cells or normal, non-activated cells. Indeed, (over-) expression of PDGFRB and/or IGF2R in various medical and experimental conditions with fibrotic pathobiology has been demonstrated for a series of diseases. (Over-) expression of PDGFRB is apparent in human liver fibrosis (Lambrecht et al, EBioMedicine, 2019; 43:501-512). (Over-) expression of IGF2R is also seen in experimental liver fibrosis (Van Beuge et al, Pharm. Res., 2011; 28:2045-54; Greupink et al, Pharm. Res., 2006; 23:1827-34). (Over-) expression of PDGFRB is demonstrated in human glomerular and interstitial renal fibrosis (Boor et al, EMBO Mol. Med., 2020; 12: e11021). (Over-) expression of PDGFRB is seen in cases of idiopathic and secondary pulmonary hypertension (Overbeek et al, Arthritis Res. Ther. 2011; 13: R61). (Over-) expression of PDGFRB is apparent in human idiopathic pulmonary fibrosis (Yamaguchi et al, Biochem. Biophys. Res. Commun. 2020; 528:269-275). (Over-) expression of PDGFRB is apparent in scleroderma/systemic sclerosis (Klareskog et al, Arthritis Rheum. 1990; 33:1534-41). (Over-) expression of PDGFRB is seen in inflammatory bowel disease (Yamaguchi et al, Tohoku J. Exp. Med. 2001; 195:21-33). (Over-) expression of PDGFRB is also apparent in some sclerosing malignant tumors, such as, amongst others, some tumors of the lung, colon, breast or prostate (Paulsson et al, Am. J. Pathol. 2009; 175:334-341).

An aspect of the invention relates to a binding molecule comprising at least two single variable antibody domains and at least one diagnostic molecule or therapeutic molecule, wherein the at least two single variable antibody domains are able to specifically bind to a transmembrane receptor expressed on a fibrogenic effector cell, which fibrogenic effector cell is characterized by the expression of alpha smooth muscle actin and/or stress fibers discernable by immune fluorescence microscopy, wherein the at least two single variable antibody domains are variable domains of a heavy chain only antibody (VHH), wherein the transmembrane receptor is an insulin-like growth factor 2 receptor (IGF2R) or wherein the transmembrane receptor is a platelet-derived growth factor beta receptor (PDGFRB). An aspect of the invention relates to a binding molecule comprising at least two single variable antibody domains and at least one diagnostic molecule or therapeutic molecule, wherein the at least two single variable antibody domains are able to specifically bind to a transmembrane receptor expressed on a fibrogenic effector cell, which fibrogenic effector cell is characterized by the expression of alpha smooth muscle actin and/or stress fibers discernable by immune fluorescence microscopy, such as any one or more of an activated: portal fibroblast, hepatic stellate cell, pericyte, alveolar smooth muscle cell, alveolar fibroblast, interstitial fibroblast, mesangial cell, dermal fibroblast, mucosal smooth muscle cell, intestinal (myo-) fibroblast, interstitial cells of Cajal, or any precursor of a tumor stromal fibroblast in cancer, preferably a fibrogenic effector cell selected from any one or more of an activated: alveolar smooth muscle cell in the lung, alveolar fibroblast in the lung and/or pericyte in the lung, interstitial fibroblast in the kidney, pericyte in the kidney and/or mesangial cell in the kidney, dermal fibroblast in the skin and/or pericyte in the skin, mucosal smooth muscle cell in the intestine, intestinal (myo-) fibroblast in the intestine, interstitial cell of Cajal in the intestine, or pericyte in the intestine, or any precursor of a tumor stromal fibroblast in cancer, wherein the at least two single variable antibody domains are variable domains of a heavy chain only antibody (VHH), wherein the transmembrane receptor is an insulin-like growth factor 2 receptor (IGF2R) or wherein the transmembrane receptor is a platelet-derived growth factor beta receptor (PDGFRB). Such a fibrogenic effector cell is characterized by the expression of alpha smooth muscle actin and/or stress fibers discernable by (immune fluorescence) microscopy or similar methods, preferably by immune fluorescence microscopy.

An embodiment is the binding molecule of the invention, wherein the at least one, preferably at least two, more preferably two, single variable antibody domain(s) is/are able to specifically bind to a transmembrane receptor expressed on a fibrogenic effector cell selected from any one or more of: an activated portal fibroblast or hepatic stellate cell in liver fibrosis, an activated alveolar fibroblasts or -smooth muscle cell in lung fibrosis, an activated mesangial or interstitial fibroblast in kidney fibrosis, an activated dermal fibroblast in systemic sclerosis/scleroderma, an activated intestinal fibroblast in chronic bowel inflammation, or an activated tumor stromal fibroblasts. The binding molecules are preferably bound to a drug or a cytotoxic molecule via, e.g., a linker comprising a Pt(II) transition metal complex or more conventional means of chemical binding such as maleimidocaproyl-based conjugation. In particular when the drug is a kinase inhibitor, such as a RHO-kinase, a JAK-2 inhibitor, or a neprilysin inhibitor, or a cytotoxic substance, a binding molecule according to the invention preferably provides to activated fibrogenic effector cells an overall reduction in contraction, an overall reduction in chemotaxis, and an overall reduction in pro-fibrotic activity such as the expression and secretion of extracellular matrix components, and chemo-attractants. This in turn will result in the attenuation and possibly reversal of the fibrotic process in question. The linker-based binding of the pharmacologically active small molecule drugs to the binding molecules effectively enforces specific delivery of the pharmacologically active small molecule drugs to activated myo-fibroblasts, contributing to reduced (off-target) toxicity which is generally seen with these kinase inhibitors and cytotoxic substances.

In said medical conditions, selective delivery of a drug to (activated) cells that play a crucial role in the onset, maintenance and exacerbation of said condition, e.g. activated hepatic stellate cells in liver fibrosis, activated alveolar fibroblasts or -smooth muscle cells in lung fibrosis, activated mesangial or interstitial fibroblasts in kidney fibrosis, activated dermal fibroblasts in systemic sclerosis/scleroderma, activated intestinal fibroblasts in chronic bowel inflammation, or even activated tumor stromal fibroblasts, is an interesting venue to pursue. The targeting of receptors such as PDGFRB and IGF2R, and preferably PDGFRB and IGF2R, that are upregulated on activated fibrogenic effector cells as compared to their normal, quiescent progenitors such as portal fibroblasts, hepatic stellate cells and pericytes in the liver, alveolar smooth muscle cells, alveolar fibroblasts or pericytes in the lung, interstitial fibroblasts, pericytes or mesangial cells in the kidney, dermal fibroblasts or pericytes in the skin, mucosal smooth muscle cells, intestinal (myo) fibroblasts, interstitial cells of Cajal, or pericytes in the intestine, or any precursor of tumor stromal fibroblasts in cancer, can be used to arrive at an effective medicinal treatment of said medical conditions or can be used prophylactically for said medical conditions. In the present invention, the targeting of PDGFRB and/or IGF2R (preferably either PDGFRB or IGF2R) is considered an important means as, through said targeting, the internalisation of a proteinaceous molecule comprising e.g. a Rho-kinase inhibitor, a JAK-2 kinase inhibitor, a neprilysin inhibitor, or a cytotoxic molecule is induced.

An aspect of the invention relates to a nucleic acid that encodes amino acid residues of at least part of a binding molecule of the invention, and preferably the amino acid residues of the binding molecule according to the invention. That is to say, the nucleic acid preferably encodes the amino acid residues of the binding molecule according to the invention, wherein the transmembrane receptor is an IGF2R, and wherein the binding molecule comprises a first VHH able to specifically bind to IGF2R and comprises a second VHH able to specifically bind to IGF2R, or wherein the transmembrane receptor is a PDGFRB, and wherein the binding molecule comprises a first VHH able to specifically bind to PDGFRB and comprises a second VHH able to specifically bind to PDGFRB. An aspect of the invention relates to a host cell for expression of amino acid residues of at least part of a binding molecule of the invention, and preferably the amino acid residues of the binding molecule according to the invention, comprising a nucleic acid according to the invention.

Also provided is a method for producing a binding molecule according to the invention, comprising culturing a host cell according to the invention, allowing for expression of at least part of said binding molecule, harvesting the binding molecule, and coupling the therapeutic or diagnostic molecule to said part of said binding molecule, optionally through a linker as defined above. An aspect of the invention relates to a method for producing a binding molecule according to the invention, comprising culturing a host cell of the invention, allowing for expression of amino acid residues of at least part of said binding molecule, preferably the amino acid residues of the binding molecule of the invention, harvesting the expressed amino acid residues of the at least part of the binding molecule or of the binding molecule, and coupling the therapeutic molecule or the diagnostic molecule to said amino acid residues of part of said binding molecule or of the binding molecule, optionally through a linker.

An aspect of the invention relates to the binding molecule according to the invention for use as a medicament. An aspect of the invention relates to the binding molecule according to the invention, for use in a method for the prevention and/or treatment of a disease or disorder associated with or characterized by the upregulation of PDGFRB and/or IGF2R expression.

As the binding molecule according to the invention comprising a therapeutic molecule is particularly suited for therapy of said medical conditions such as liver fibrosis, liver cirrhosis, lung fibrosis, kidney fibrosis, systemic sclerosis/scleroderma, chronic bowel inflammation, and cancer, preferably lung fibrosis, kidney fibrosis, systemic sclerosis/scleroderma, chronic bowel inflammation, and cancer, more preferably lung fibrosis and kidney fibrosis or systemic sclerosis/scleroderma, chronic bowel inflammation and cancer, the invention further provides a pharmaceutical composition comprising at least one binding molecule according to the invention and at least one pharmaceutically acceptable excipient. An aspect of the invention relates to a pharmaceutical composition comprising at least one binding molecule according to the invention and at least one pharmaceutically acceptable excipient. Preferably, the pharmaceutical composition comprises one binding molecule of the invention, or two. Such pharmaceutical composition may further comprise at least one other compound useful in the treatment of said medical conditions. An aspect of the invention relates to the pharmaceutical composition of the invention, for use as a medicament. An aspect of the invention relates to the pharmaceutical composition of the invention, for use in a method for the prevention and/or treatment of a disease or disorder associated with or characterized by the upregulation of PDGFRB and/or IGF2R expression. A pharmaceutical composition according to the invention is particularly useful as an acute treatment for secondary or tertiary complications of these conditions, e.g. to provide immediate relief of portal hypertension and its acute complications such as oesophageal bleeding in the case of end-stage liver cirrhosis, or the immediate relief of pulmonary hypertension and its acute complications such as cardiac failure in the case of end-stage pulmonary fibrosis. An aspect of the invention relates to a pharmaceutical composition comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor. An aspect of the invention relates to the pharmaceutical composition of the invention comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor, for use as an adjuvant treatment of secondary hemodynamic manifestations of fibrotic disease, such as portal hypertension and pulmonary hypertension. An aspect of the invention relates to the pharmaceutical composition of the invention comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor, for use as an adjuvant treatment of secondary hemodynamic manifestations of fibrotic disease, such as portal hypertension and pulmonary hypertension, wherein said use is in the treatment of an acute manifestation of: portal hypertension such as acute esophageal bleeding or pulmonary hypertension such as acute cardiac decompensation.

As the binding molecule according to the invention specifically binds to transmembrane receptors that are selectively over-expressed by fibrogenic effector cells, a binding molecule according to the invention comprising a diagnostic molecule such as a radio-tracer, is particularly suited for diagnosis of said medical conditions. The invention thus also provides a diagnostic composition comprising at least one binding molecule according to the invention comprising a diagnostic molecule, preferably an imaging agent, and a diluent and/or excipient. An aspect of the invention relates to a diagnostic composition comprising at least one binding molecule of the invention comprising a diagnostic molecule, wherein the diagnostic molecule is an imaging agent, and comprising a diluent and/or excipient. An aspect of the invention relates to the use of the binding molecule of the invention comprising a diagnostic molecule, wherein the diagnostic molecule is an imaging agent, or to the use of the diagnostic composition of the invention, in a method for diagnosing of a disease or disorder associated with or characterized by the upregulation of PDGFRB and/or IGF2R expression.

Particularly preferred binding molecules of the invention are biparatopic binding molecules comprising either a first and a second VHH domain binding to IGF2R, or comprising a first and a second VHH domain binding to PDGFRB. Preferably, such binding molecule comprises a half-life extender, preferably an albumin binding domain (ABD), for example an albumin binding VHH. For a preferred binding molecule of the invention, the therapeutic molecule is a kinase inhibitor, preferably selected from Rho-kinase inhibitors, JAK-2 inhibitors or neprilysin inhibitors, or Angiotensin II Receptor antagonists, such as losartan. More preferably, for the binding molecule of the invention the therapeutic molecule is a Rho-kinase inhibitor or a JAK-2 inhibitor. According to the invention, typically suitable therapeutic molecules for conjugating to the VHH domains of the binding molecule are Y27632 which is a ROCK (Rho-kinase) inhibitor, and pacritinib which is a JAK-2 inhibitor. For the binding molecule of the invention comprising a therapeutic molecule, the ratio between the therapeutic molecule and the two VHH domains can be 1 to 1 or for example any ratio selected from 2:1 and 64:1, such as 3:1, 4:1, 5:1, 6:1, 7:1, 8:1, 10:1, 12:1, 16:1, 20:1, 24:1, 30:1, 32:1, 40:1, 50:1 and any ratio in between. That is to say, a binding molecule comprises a single copy of the therapeutic molecule, or a binding molecule comprises multiple copies of the therapeutic molecule, such as two, three, four, six, eight, ten, twelve, sixteen copies.

Preferred biparatopic binding molecules of the invention, comprising two VHH domains, are 13E8-13F11, 13F11-13E8, 13E8-13F11-ABD, 13F11-13E8-ABD, 13E8-13F11 (Y27632), 13E8-13F11-ABD (Y27632), 13E8-13F11 (pacritinib), 13E8-13F11-ABD (pacritinib), 13F11-13E8 (Y27632), 13F11-13E8-ABD (Y27632), 13F11-13E8 (pacritinib), 13F11-13E8-ABD (pacritinib), comprising optionally (and preferred) an albumin binding domain (ABD) and optionally (and preferred) a therapeutic molecule such as Y27632 or pacritinib as indicated. Preferred are 13E8-13F11-ABD (pacritinib), 13E8-13F11-ABD (Y27632), 13F11-13E8-ABD (Y27632) and 13F11-13E8-ABD (pacritinib), more preferred are 13F11-13E8 (Y27632), 13F11-13E8-ABD (Y27632), 13F11-13E8 (pacritinib) and 13F11-13E8-ABD (pacritinib). Particularly preferred is the binding molecule 13F11-13E8 (Y27632) or 13F11-13E8-ABD (Y27632).

Preferred biparatopic binding molecules of the invention, comprising two VHH domains, are SP02P-SP26P, SP26P-SP02P, SP02P-SP26P-ABD, SP26P-SP02P-ABD, SP02P-SP26P(Y27632), SP02P-SP26P-ABD (Y27632), SP02P-SP26P (pacritinib), SP02P-SP26P-ABD (pacritinib), SP26P-SP02P(Y27632), SP26P-SP02P-ABD (Y27632), SP26P-SP02P (pacritinib), SP26P-SP02P-ABD (pacritinib), comprising optionally (and preferred) an albumin binding domain (ABD) and optionally (and preferred) a therapeutic molecule such as Y27632 or pacritinib as indicated. Preferred are SP02P-SP26P-ABD (Y27632), SP02P-SP26P-ABD (pacritinib), SP26P-SP02P-ABD (Y27632) and SP26P-SP02P-ABD (pacritinib), more preferred are SP02P-SP26P(Y27632), SP02P-SP26P-ABD (Y27632), SP02P-SP26P (pacritinib) and SP02P-SP26P-ABD (pacritinib).

Surprisingly, the inventors provided relatively high-affinity biparatopic binding molecules comprising a first and a second VHH domain, wherein the high affinity was apparent despite of the respective lower binding affinities of the two corresponding monomeric VHHs to respective substrates (target receptor), demonstrating a synergistic contribution of each of the two monomeric VHHs to the binding characteristics of the resulting biparatopic constructs, and hence superiority of the biparatopic design over monomeric VHHs. In particular the synergy is apparent for binding molecules targeting the IGF2R, such as binding molecules comprising VHH domains 13E8 and 13F11.

### BRIEF DESCRIPTION OF THE FIGURES

FIG. 1. Overview of the research process of (A) creating VHH libraries from blood of immunized llamas to (B) enrichment for binding VHH sequences through phage display selection and (C) final selection of binding clones for further characterization (figure adapted from Schoonooghe S. et al, Immunobiology 2012, 217:1266-1272).

FIG. 2: Binding assay of selected IGF2R-binding VHHs to human IGF2R ectodomain (ECD).

FIG. 3. Phage outputs from the first selection round of the PDGFRB libraries on human PDGFRB ectodomain (libraries 152 and 153) and rat PDGFRB ectodomain (libraries 154 and 155).

FIG. 4. Phage outputs from the second selection round of the PDGFRB libraries on SCC VII cells transfected with either human PDGFRB (libraries 152 and 153), or rat PDGFRB (libraries 154 and 155).

FIG. 5. Amino acid sequence of human PDGFRB-binding VHHs (CDRs underlined). Affinities are measured on human PDGFRB ectodomain (hPDGFRB-ECD), SCC VII cells transfected with human PDGFRB receptor (SCC-hPDGFRB) and the human hepatic stellate cell line (LX-2). Data of the competition assays is included to categorize the VHHs in different epitope groups.

FIG. 6. Amino acid sequence of rat PDGFRB-binding VHHs (CDRs underlined). Affinities are measured on rat PDGFRB ectodomain (rPDGFRB-ECD) and squamous cell carcinoma cells transfected with rat PDGFRB receptor (SCC-rPDGFRB). Data of the competition assays is included to categorize the VHHs in different epitope groups.

FIG. 7. Schematically displayed vector for the expression of monomeric VHH or VHH constructs. Expression cassette is expanded.

FIG. 8. Construction and characterization of bivalent and biparatopic VHH constructs directed against human IGF2R. A. Schematic representation of monovalent, bivalent and biparatopic VHH formats. In the bivalent and biparatopic formats, two VHHs are fused with a flexible (Gly-Gly-Gly-Gly-Ser) 3 linker (GGGGSGGGGSGGGGS; SEQ ID NO: 369). All formats contain a C-terminal cysteine for site-directed conjugation to thiol-reactive probes and a hexa-histidine-tag (HHHHHH; SEQ ID NO: 370) or EPEA-tag (EPEA; SEQ ID NO: 371) for affinity chromatography purification. B. Binding of Alexa Fluor 647-conjugated VHHs and VHH constructs to immobilized recombinant human IGF2R ectodomain (ECD). C. Binding of IRDye 800-(IRD800-) conjugated VHHs and VHH constructs to immobilized recombinant human IGF2R ectodomain (ECD). The R2-IRD800 construct is based on a non-binding VHH termed R2 (negative control). D. Bₘₐₓ and K_{D}+95% confidence intervals for selected VHHs.

FIG. 9. Binding of selected VHHs to PDGFRB-expressing cells. A) Binding of VHHs selected from libraries 1 and 2 to SCC VII cells transgenically expressing full-length human PDGFRB (SCC-hPDGFRB). B) Binding of VHHs selected from libraries 3 and 4 to SCC VII cells transgenically expressing full-length rat PDGFRB (SCC-rPDGFRB). In the binding assay, cell-bound VHH were detected using anti-VHH rabbit serum and IRDye800-conjugated donkey-anti-rabbit secondary antibody.

FIG. 10. Competition enzyme linked immunosorbent assay (ELISA) with VHHs directed against human IGF2R.

FIG. 11. A) Competition assay similar to the assay displayed in FIG. 10, with IRDye800-conjugated SP02P as the reporter VHH, and unconjugated SP01P, SP02P, SP05P, SP07P, SP13P, SP14P or SP20P as competitor VHH. B) Competition enzyme linked immunosorbent assay (ELISA) assay with IRDye800-conjugated SP26P as the reporter VHH, and unconjugated SP02P and SP26P as competitor VHHs, on human PDGFRB ECD.

FIG. 12. A) Binding of IRDye800-conjugated 13F11-13E8 (F11E8) to SCC cells transgenically expressing hIGF2R or hIGF2R. B-D) Binding of IRDye800-conjugated 13F11-13E8 (F11E8) and IRDye800-conjugates of its corresponding monomeric VHHs F11 and E8, to SCC cells transgenically expressing murine-(B), human-(D) or rat (C) IGF2R.

FIG. 13. Binding of 13F11-13E8-ABD (F11E8-ABD) to IGF2R and serum albumin to promote in vivo serum half life. A) Binding assay on recombinant human IGF2R ECD. B) Binding assay on serum albumin from different species. C) Serum levels of F11E8-ABD and F11E8 in rats after intravenous bolus injection (serum concentrations were assessed by ELISA with LOD of approximately 20 nM). D) Bio-distribution of radiolabeled F11E8 (upper) and F11E8-ABD (lower) in rats assessed at various time points after injection by means of whole-body Positron Emission Tomography (PET).

FIG. 14. Binding of 13F11-13E8-ABD conjugated to Alexa Fluor 488 (FE-A-A488) to activated primary rat hepatic stellate cells.

FIG. 15. Binding of PDGFRB-directed VHHs and biparatopic VHH constructs to PDGFRB. A) Binding of selected monomeric VHHs to human PDGFRB ECD. B) Binding of the biparatopic VHH construct SP28-SP26P to cells transgenically expressing human PDGFRB, rat PDGFRB, or human IGF2R, the latter serving as a negative control. C) Binding assay of biparatopic VHH SP02-SP26P on cells transgenically expressing human PDGFRB, rat PDGFRB, or human IGF2R, the latter serving as a negative control. D) Binding of IRDye800-conjugated SP02P-SP26P, and corresponding monomeric VHHs SP02P and SP26P, to murine PDGFRB ECD (left panel), rat PDGFRB ECD (middle panel), or human PDGFRB ECD (right panel).

FIG. 16. Internalization of IGF2R-binding VHHs and VHH constructs as assessed by fluorescence microscopy after acid wash (to remove non-internalized VHHs and VHH constructs). Pictures show internalized A647-conjugated 13F11 (13F11-A647) and 13F11-13E8 (13F11-13E8-A647) in various cells. A) A549 cells that endogenously express IGF2R. B) NIH 3T3 2.2 cells that transgenically express human IGF2R. C) Mock transfected NIH 3T3 2.2 cells not expressing IGF2R. D) Validation of the acid wash step for removal of surface-bound VHHs, executed on A549 cells.

FIG. 17. Internalization of IGF2R-binding VHH and VHH constructs as assessed by fluorescence internalization assay. A) Internalization of IRDye800-conjugated 13F11-13E8 (F11E8-IRD800) and its reciprocal 13E8-13F11 (E8F11-IRD800) on SCC cells transgenically expressing human IGF2R. B) Internalization of IRDye800-conjugated 13F11-13E8 (F11E8-IRD800) and its reciprocal 13E8-13F11 (E8F11-IRD800) on SCC cells transgenically expressing rat IGF2R. C) Internalization of IRDye800-conjugated 13F11-13E8 (F11E8-IRD800) and the corresponding monomeric VHHs 13F11 (F11-IRD800) and 13E8 (E8-IRD800) on SCC cells transgenically expressing murine IGF2R (left panel), rat IGF2R (middle panel), or human IGF2R (right panel).

FIG. 18. Internalization of biparatopic VHH constructs 13F11-13E8 (upper series of panels), 13E8-13F11 (middle series of panels) and the monovalent VHH 13F11 (lower series of panels) into human hepatic stellate cell-derived LX2 cells. VHH and VHH constructs were conjugated to the pH sensitive pHrodo red dye in order to visualize internalization by fluorescence microscopy.

FIG. 19. Internalization of SP02P-SP26P into cells transgenically expressing human PDGFRB (SCC-hPDGFRB, left panel) or endogenously expressing human PDGFRB (LX-2, right panel).

FIG. 20. Internalization of monovalent (SP02 or SP26) and biparatopic (SP02-SP26) VHHs conjugated to IRDye800 into SCC cells transgenically expressing rat PDGFRB (left panel) or human PDGFRB (right panel).

FIG. 21. Contraction assay on activated hepatic stellate cells (HSCs). Depicted is the measured area of the gel after 6 days of incubation with the NDCs. The contraction of the LX-2 cells without any inhibitor was set at 0% relaxation, while 1 μM BDM was set at 100% relaxation. Note: for the medium no contraction inhibition was observed.

FIG. 22. Expression of IGF2R in livers of cirrhotic rats (bile duct ligation; BDL) and healthy control rats (sham) as assessed by Immunofluorescence microscopy using A647-conjugated 13F11-13E8-ABD as the fluorescent probe.

FIG. 23. Expression of PDGFRB in livers of cirrhotic rats (bile duct ligation; BDL) and healthy control rats (sham) as assessed by Immunofluorescence microscopy using A647-conjugated SP02P-SP26P as the fluorescent probe.

FIG. 24. Change in portal pressure (PP) and mean arterial pressure (MAP) in BDL rats as a function of minutes after dosing for A) 13F11-13E8-ABD-Y27632 (test conjugate) and B) 13F11-13E8-ABD-ethyl (mock conjugate).

FIG. 25. In vivo targeting of IGF2R by the biparatopic VHH construct 13F11-13E8-ABD-Y27632 (test conjugate) in BDL rats and not in healthy SHAM controls. In situ detection of the infused test conjugate. Liver cryo-sections of BDL rats (left) and SHAM rats (right) probed for the presence of test conjugate with a VHH-specific detection antibody. The test conjugate is clearly detected in the cellular layers lining the obstructed sinusoids in the cirrhotic liver of BDL rats, and not in the healthy liver of SHAM rats.

### DETAILED DESCRIPTION OF THE INVENTION

First, it should be noted that all steps, methods, and techniques that are not specifically described in detail can be performed in a manner known per se, as will be clear to the skilled person, unless indicated otherwise. Reference is made to standard handbooks and the common general knowledge mentioned herein describing techniques for protein engineering, such as affinity maturation and other techniques for improving the specificity and other desired properties of binding moieties, present in a binding molecule according to the invention, such as immunoglobulin single variable domain antibody (ISVD), a variable domain of a heavy chain (VH), a variable domain of a heavy chain only antibody (VHH), a domain antibody (dAb), or a single domain antibody (sdAb). It must be noted that as used herein, the singular forms “a”, “an”, and “the” include plural references unless the context clearly indicates otherwise. Those skilled in the art will recognize or be able to ascertain using no more than routine experimentation, many equivalents to the specific embodiments of the invention described herein. Such equivalents are intended to be encompassed by the present invention. The term “and/or” wherever used herein includes the meaning of “and”, “or” and “all or any other combination of the elements connected by said term”. The term “about” or “approximately” as used herein means within 25%, preferably within 20%, more preferably within 15%, and most preferably within 10% of a given value or range. Throughout this specification and the claims which follow, unless the context requires otherwise, the word “comprise” and variations such as “comprises” and “comprising” will be understood to imply the inclusion of the stated feature, but not the exclusion of any other feature. When used herein the term “comprising” can be substituted with the term “containing” or “including” or “having”. In contrast, the word “consisting of”, in combination with (a) feature(s) means the inclusion of the feature(s) mentioned but the exclusion of any other feature(s).

Second, with “fibrotic afflictions”, “fibrotic medical conditions”, “fibrotic disease”, and “fibrosis” are meant those clinically recognized afflictions of which the pathobiology is known, or is reported to have, a causative, putative, or strongly suspected fibrogenic component. These afflictions include for example liver (or hepatic-) fibrosis and liver cirrhosis, lung fibrosis (or pulmonary fibrosis), kidney fibrosis (or renal fibrosis), systemic sclerosis/scleroderma, inflammatory bowel disease and/or certain sclerosing cancerous malignancies. These fibrotic afflictions also include any (secondary) clinical manifestations that are attributable to said fibrotic afflictions, such as portal hypertension or pulmonary (arterial and/or venous) hypertension in the cases of liver fibrosis and/or cirrhosis, and pulmonary fibrosis, respectively. Furthermore, these fibrotic afflictions also include any acute complications of such (secondary) clinical manifestations, such as oesophageal bleeding or cardiovascular or pulmonary failure in the cases of portal hypertension and pulmonary (arterial and/or venous) hypertension, respectively.

Third, with a ‘fibrogenic effector cell’ is meant those cells that are recognized mediators of fibrosis due to their activated phenotype which includes contractile, chemotactic and pro-fibrotic activity, such as enhanced extracellular matrix (ECM) protein production (e.g. collagen). Phenotypically, these cells can be characterized as mature activated myo-fibroblasts, typically expressing hallmark proteins such as alpha smooth muscle actin (αSMA), and typically displaying (e.g. actin-) stress fibers upon (immune fluorescent) microscopic evaluation.

Fourth, with “precursors of fibrogenic effector cells” are meant those, usually organ-specific, and usually mesenchymal, cells which through activation and trans-differentiation give rise to said fibrogenic effector cells, such as portal fibroblasts, hepatic stellate cells (HSC) and pericytes in the liver, alveolar smooth muscle cells (SMC), alveolar fibroblasts or pericytes in the lung, interstitial fibroblasts, pericytes or mesangial cells in the kidney, dermal fibroblasts or pericytes in the skin, mucosal smooth muscle cells, intestinal (myo-) fibroblasts, interstitial cells of Cajal, or pericytes in the intestine, or any precursor of tumor stromal fibroblasts in cancer. See for descriptions of such cell types and their activation in the context of organ fibrosis Klinkhammer et al. (Mol. Aspects Med., 2017; 62:44-62), Tsuchida et al. (Nat. Rev. Gastroenterol. Hepatol. 2017; 14:397-411), Boor et al. (Nat. Rev. Nephrol. 2010; 6:643-56) and Yamaguchi et al. (Biochem. Biophys. Res. Commun. 2020; 528:269-275).

In an embodiment, the invention provides a binding molecule comprising at least one single variable antibody domain, preferably at least two, such as two, single variable antibody domains, and at least one diagnostic or therapeutic molecule, wherein the at least one or at least two, preferably two single variable antibody domain(s) is/are able to specifically bind to a transmembrane receptor expressed on fibrogenic effector cells, which cells are preferably characterized by increased proliferation, contractility, chemotaxis, and enhanced extracellular matrix (ECM) protein production (e.g. collagen) as compared to their progenitors.

An aspect of the invention relates to a binding molecule comprising at least one single variable antibody domain, preferably at least two, such as two, single variable antibody domains, and at least one diagnostic or therapeutic molecule, wherein the at least one or at least two, preferably two single variable antibody domain(s) is/are able to specifically bind to a transmembrane receptor expressed on a fibrogenic effector cell.

An embodiment is the binding molecule of the invention, wherein the at least one single variable antibody domain, preferably at least two, such as two, single variable antibody domains, is/are able to specifically bind to a transmembrane receptor expressed on a fibrogenic effector cell. An aspect of the invention relates to a binding molecule comprising at least two single variable antibody domains and at least one diagnostic molecule or therapeutic molecule, wherein the at least two single variable antibody domains are able to specifically bind to a transmembrane receptor expressed on a fibrogenic effector cell, which fibrogenic effector cell is characterized by the expression of alpha smooth muscle actin and/or stress fibers discernable by immune fluorescence microscopy, wherein the at least two single variable antibody domains are variable domains of a heavy chain only antibody (VHH), wherein the transmembrane receptor is an insulin-like growth factor 2 receptor (IGF2R) or wherein the transmembrane receptor is a platelet-derived growth factor beta receptor (PDGFRB).

An aspect of the invention relates to a binding molecule comprising at least one single variable antibody domain, preferably at least two, such as two, single variable antibody domains, and at least one diagnostic molecule or therapeutic molecule, wherein the at least one or at least two, preferably two single variable antibody domain(s) is/are able to specifically bind to a transmembrane receptor expressed on a fibrogenic effector cell, which fibrogenic effector cell is characterized by the expression of alpha smooth muscle actin and/or stress fibers discernable by immune fluorescence microscopy, such as any one or more of an activated: portal fibroblast, hepatic stellate cell, pericyte, alveolar smooth muscle cell, alveolar fibroblast, interstitial fibroblast, mesangial cell, dermal fibroblast, mucosal smooth muscle cell, intestinal (myo-) fibroblast, interstitial cells of Cajal, or any precursor of a tumor stromal fibroblast in cancer, preferably a fibrogenic effector cell selected from any one or more of an activated: alveolar smooth muscle cell in the lung, alveolar fibroblast in the lung and/or pericyte in the lung, interstitial fibroblast in the kidney, pericyte in the kidney and/or mesangial cell in the kidney, dermal fibroblast in the skin and/or pericyte in the skin, mucosal smooth muscle cell in the intestine, intestinal (myo-) fibroblast in the intestine, interstitial cell of Cajal in the intestine, or pericyte in the intestine, or any precursor of a tumor stromal fibroblast in cancer. A therapeutic molecule comprised within a binding molecule according to the invention preferably induces relaxation of a fibrogenic effector cell, which is a sign of diminished activation, thereby decreasing and, without wishing to be bound by any theory, also reversing the overall fibrogenic process. One example of such therapeutic molecule is Y27632, a small molecule kinase inhibitor which has been shown to induce relaxation of activated hepatic stellate cells (HSC) in a working example of the current invention. In particular, in another working example, it was demonstrated that a binding molecule of the invention in particular interacts with the receptors on activated HSC in vivo, and induced selective lowering of portal hypertension in vivo. The examples demonstrate a proof of concept that a binding molecule of the invention can be used to counter fibrotic afflictions. Because of the well-established scientific knowledge that fibrogenic effector cells such as activated HSCs are pivotal effectors in the majority of fibrotic afflictions, the binding molecule of the invention holds therapeutic potential in all of such fibrotic afflictions, in any of their (secondary) clinical manifestations, and in acute complications of any of such (secondary) clinical manifestations.

An aspect of the invention relates to a binding molecule comprising at least two single variable antibody domains and at least one diagnostic molecule or therapeutic molecule, wherein the at least two single variable antibody domains are able to specifically bind to a transmembrane receptor expressed on a fibrogenic effector cell, which fibrogenic effector cell is characterized by the expression of alpha smooth muscle actin and/or stress fibers discernable by immune fluorescence microscopy, such as any one or more of an activated: portal fibroblast, hepatic stellate cell, pericyte, alveolar smooth muscle cell, alveolar fibroblast, interstitial fibroblast, mesangial cell, dermal fibroblast, mucosal smooth muscle cell, intestinal (myo-) fibroblast, interstitial cells of Cajal, or any precursor of a tumor stromal fibroblast in cancer, preferably a fibrogenic effector cell selected from any one or more of an activated: alveolar smooth muscle cell in the lung, alveolar fibroblast in the lung and/or pericyte in the lung, interstitial fibroblast in the kidney, pericyte in the kidney and/or mesangial cell in the kidney, dermal fibroblast in the skin and/or pericyte in the skin, mucosal smooth muscle cell in the intestine, intestinal (myo-) fibroblast in the intestine, interstitial cell of Cajal in the intestine, or pericyte in the intestine, or any precursor of a tumor stromal fibroblast in cancer, wherein the at least two single variable antibody domains are variable domains of a heavy chain only antibody (VHH), wherein the transmembrane receptor is an insulin-like growth factor 2 receptor (IGF2R) or wherein the transmembrane receptor is a platelet-derived growth factor beta receptor (PDGFRB). Preferred is the binding molecule comprising at least two single variable antibody domains and at least one diagnostic molecule or therapeutic molecule of the invention, wherein the fibrogenic effector cell is characterized by the expression of alpha smooth muscle actin and/or stress fibers discernable by immune fluorescence microscopy, such as any one or more of an activated: portal fibroblast, hepatic stellate cell, pericyte, alveolar smooth muscle cell, alveolar fibroblast, interstitial fibroblast, mesangial cell, dermal fibroblast, mucosal smooth muscle cell, intestinal (myo-) fibroblast, interstitial cells of Cajal, or any precursor of a tumor stromal fibroblast in cancer, more preferably a fibrogenic effector cell selected from any one or more of an activated: alveolar smooth muscle cell in the lung, alveolar fibroblast in the lung and/or pericyte in the lung, interstitial fibroblast in the kidney, pericyte in the kidney and/or mesangial cell in the kidney, dermal fibroblast in the skin and/or pericyte in the skin, mucosal smooth muscle cell in the intestine, intestinal (myo-) fibroblast in the intestine, interstitial cell of Cajal in the intestine, or pericyte in the intestine, or any precursor of a tumor stromal fibroblast in cancer.

A binding molecule of the current invention must be able to specifically bind to a transmembrane receptor expressed on fibrogenic effector cells. Examples of receptors that are expressed in sufficient amount on fibrogenic effector cells are, e.g., platelet-derived growth factor receptor beta (PDGFRB) and insulin-like growth factor 2 receptor (IGF2R, also known as cationic-independent mannose-6-phosphate receptor). Hence, in a preferred embodiment, a binding molecule according to the invention is provided, wherein the transmembrane receptor is a PDGFRB or an IGF2R. Certain embodiments relate to the binding molecule according to the invention, wherein the transmembrane receptor is an insulin-like growth factor 2 receptor (IGF2R). An embodiment is the binding molecule according to the invention, wherein the transmembrane receptor is a platelet-derived growth factor beta receptor (PDGFRB).

PDGF is one of numerous growth factors that regulate cell growth and division. In particular, PDGF plays a significant role in blood vessel formation, the growth of blood vessels from already existing blood vessel tissue, mitogenesis, i.e. proliferation, of mesenchymal cells such as fibroblasts, osteoblasts, tenocytes, vascular smooth muscle cells and mesenchymal stem cells as well as chemotaxis, the directed migration, of mesenchymal cells. PDGF is a dimeric glycoprotein that can be composed of two A subunits (PDGF-AA), two B subunits (PDGF-BB), or one of each (PDGF-AB). PDGF is a potent mitogen (a protein that encourages a cell to commence cell division, triggering mitosis) for cells of mesenchymal origin, including fibroblasts, smooth muscle cells and glial cells. In both mouse and human, the PDGF signalling network consists of five ligands, PDGF-AA, -BB, -AB, -CC, and -DD, and two receptors, PDGFR-alpha (PDGFRA) and PDGFR-beta (PDGFRB). All PDGFs function as secreted, disulphide-linked homodimers, but only PDGFA and PDGFB can form functional heterodimers. PDGF is overly synthesized upon activation of the progenitors of fibrogenic effector cells as may occur in conditions of organ tissue damage such as (and not restricted to) alcoholism or hepatitis (liver), diabetes or hypertension (kidney, lung), or chronic inflammation (bowel). As such, these progenitors are activated and transdifferentiate into fibrogenic effector cells giving rise to the genesis of connective tissue that is normally prevalent in wound healing. PDGFs are a required element in this process. In essence, the PDGFs allow a cell to skip the G1 checkpoints in order to divide. It has been shown that in monocytes-macrophages and fibroblasts, exogenously administered PDGF stimulates chemotaxis, proliferation, and gene expression and significantly augments the influx of inflammatory cells and (progenitors of) fibrogenic effector cells, accelerating extracellular matrix and collagen formation and thus reducing the time for the healing process to occur.

The mannose 6-phosphate/insulin-like growth factor-2 receptor (M6P/IGF2R) plays an important active role in early fibrogenesis by participating in the activation of latent transforming growth factor beta (TGFB), a potent activator and stimulus of (progenitors of) fibrogenic effector cells. IGF2R is a transmembrane glycoprotein which is involved in the clearance of extracellular ligands (IGF-II), activation of extracellular ligands (TGFβ) and in the sorting of newly synthesized, M6P-tagged, lysosomal enzymes from the trans-Golgi network (TGN) to lysosomes. Because of this function, IGF2R cycles between sorting endosomes, the endocytic recycling compartment, TGN, late endosomes and the plasma membrane (Maxfield & McGraw, Nat. Rev. Mol. Cell Biol. 2004 February; 5 (2): 121-32).

A binding molecule according to the invention can be used for said fibrotic afflictions, wherein fibrogenic effector cells expressing one of the transmembrane receptors described above play a role. In a working example, the invention provides proof of principle, on (activated) HSC in experimental liver fibrosis with (secondary) portal hypertension.

In particular when the binding molecule comprises a therapeutic molecule that acts inside the cell, it is preferred that the binding molecule is taken up by the cell, e.g. through ligand or antibody internalization. Ligand internalization is defined as a receptor-mediated endocytic process in which the cell will only take in an extracellular molecule (e.g., a (natural) ligand or an antibody) if it binds to its specific receptor protein on the cell's surface. Endocytosis is a process by which cells internalize non-particulate materials such as proteins or polysaccharides by engulfing them. Thus, in a preferred embodiment, a binding molecule according to the invention is provided, wherein binding of the binding molecule to the receptor enables induction of ligand or antibody internalization. An embodiment is the binding molecule according to the invention, wherein binding of the binding molecule to said receptor enables induction of ligand internalization or antibody internalization. Said receptor is either IGF2R or PDGFRB. An embodiment is the binding molecule according to the invention, wherein binding of the binding molecule to said receptor enables induction of ligand internalization or antibody internalization, and wherein binding of one of the single variable antibody domains to its antigen modulates the binding of the other single variable antibody domain to its antigen. As said, preferably, the single variable antibody domains are VHH domains according to the invention.

In a preferred embodiment, a binding molecule according to the invention is provided, wherein the binding molecule comprises an additional single variable antibody domain that is able to specifically bind to a transmembrane receptor expressed on an fibrogenic effector cells. Such binding molecule may comprise two single variable antibody domains that are able to bind two different antigens, e.g. epitopes on the same antigen, i.e., two different epitopes on one receptor. The latter is also called a biparatopic binding molecule. A binding molecule of the invention may also comprise two single variable antibody domains that are both specific for the same epitope. Such binding molecule, which is also known as bivalent binding molecule cannot bind to two epitopes on a single receptor (as generally, there are no two of those epitope sequence present in the sequence of one receptor (unless it is a dimer), but is able to bind the same epitope sequence on two receptors of the same type, e.g., two PDGF receptors that are in the vicinity of each other. The effect thereof is that the bivalent binding molecule cross-links the two receptors, thereby inducing internalization of the receptors and the binding molecule bound thereto. In case of a biparatopic binding molecule, binding results in receptor oligomerisation, which further enhances receptor internalization. In a preferred embodiment, therefore, a binding molecule according to the invention is provided, wherein the binding molecule is a multiparatopic, preferably a biparatopic binding molecule. Alternatively or combined with a multiparatopic specificity, it is preferred that the binding molecule is a multivalent, preferably a bivalent binding molecule. Alternatively, or combined with a multiparatopic specificity, a bivalent specificity, or both, it is preferred that the binding molecule is a multispecific, preferably a bispecific binding molecule. A binding molecule of the present invention can thus combine multiple single variable antibody domains, wherein, e.g., two are bivalent towards each other and, in addition, biparatopic and/or bispecific with respect to a third, fourth, fifth or even sixth single variable antibody domain. Although a binding molecule according to the invention can comprise more than six single variable antibody domains, it is preferred that a binding molecule according to the invention comprises 2-6, preferably 2-4, more preferably 2 single variable antibody domains, for ease of production and which have shown excellent results in in vitro and in vivo models, as defined previously and below.

A single variable antibody domain present in a binding molecule according to the invention can be any kind of single variable antibody domain. Classical single variable antibody domains are variable domains of (parts of) antibodies, such as a (single) domain antibody or an immunoglobulin single variable domain antibody. Preferably, the at least one single variable antibody domain and/or the additional single variable antibody domain are, independently from one another, selected from the group consisting of an immunoglobulin single variable domain antibody (ISVD), a variable domain of a heavy chain (VH), a variable domain of a heavy chain only antibody (VHH), a domain antibody (dAb), and a single domain antibody (sdAb). More preferably, both the at least one single variable antibody domain and the additional single variable antibody domain are single variable domain antibodies, preferably variable domains of heavy chain only antibodies. These smaller (parts of) antibodies are preferred, because, in particular in a tandem format (e.g. bivalent or biparatopic), they show high internalisation capacity and optimal tissue penetration because of their smaller size.

The term “immunoglobulin single variable domain” (“ISVD”), interchangeably used with “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins a heavy chain variable domain (VH) and a light chain variable domain (VL) interact to form an antigen binding site. In this case, the complementarity determining regions (CDRs) of both VH and VL will contribute to the antigen binding site, i.e. a total of six CDRs will be involved in antigen binding site formation. In contrast, the binding site of an immunoglobulin single variable domain is formed by a single VH or VL domain. Hence, the antigen binding site of an immunoglobulin single variable domain is formed by no more than three CDRs. The term “immunoglobulin single variable domain” hence does not comprise conventional immunoglobulins or their fragments which require interaction of at least two variable domains for the formation of an antigen binding site. This is also the case for embodiments of the invention which “comprise” or “contain” an immunoglobulin single variable domain. Thus, a binding molecule or a composition that “comprises” or “contains” an immunoglobulin single variable domain may relate to e.g. constructs comprising more than one immunoglobulin single variable domain. Alternatively, there may be further constituents other than the immunoglobulin single variable domains, e.g. auxiliary agents of different kinds, protein tags, colorants, dyes, etc. However, these terms do comprise fragments of conventional immunoglobulins wherein the antigen binding site is formed by a single variable domain. Generally, single variable domains will be amino acid sequences that essentially consist of four framework regions (FR1 to FR4, respectively) and three complementarity determining regions (CDR1 to CDR3, respectively). Such single variable domains and fragments most preferably comprise an immunoglobulin fold or are capable for forming, under suitable conditions, an immunoglobulin fold. As such, the single variable domain may e.g. comprise a light chain variable domain sequence (e.g. a VL-sequence) or a suitable fragment thereof; or a heavy chain variable domain sequence (e.g. a VH-sequence or a VHH sequence) or a suitable fragment thereof; as long as it is capable of forming a single antigen binding unit (i.e. a functional antigen binding unit that essentially consists of the single variable domain, such that the single antigen binding domain does not need to interact with another variable domain to form a functional antigen binding unit, as it is e.g. the case for the variable domains that are present in e.g. conventional antibodies and scFv fragments that need to interact with another variable domain—e.g. through a VH/VL interaction—to form a functional antigen binding domain). In one embodiment of the invention, the immunoglobulin single variable domains are light chain variable domain sequences (e.g. a VL-sequence) or heavy chain variable domain sequences (e.g. a VH-sequence); more specifically, the immunoglobulin single variable domains can be heavy chain variable domain sequences that are derived from a conventional four-chain antibody or heavy chain variable domain sequences that are derived from a heavy chain antibody (e.g. a VHH). For a general description of heavy chain antibodies and the variable domains thereof, reference is inter alia made to the prior art cited herein, as well as to the prior art mentioned on page 59 of WO 08/020079 and to the list of references mentioned on pages 41-43 of the International application WO 06/040153, which prior art and references are incorporated herein by reference. As described in these references, VHH sequences and partially humanized VHH sequences can in particular be characterized by the presence of one or more “Hallmark residues” in one or more of the framework sequences.

Preferred is the binding molecule according to the invention, wherein the transmembrane receptor is an IGF2R, and wherein the binding molecule comprises a first VHH able to specifically bind to IGF2R and comprises a second VHH able to specifically bind to IGF2R.

Typically, for the binding molecule according to the invention, wherein the transmembrane receptor is an IGF2R, the binding molecule comprises at least two single variable antibody domains, preferably two single variable antibody domains, that are, independently from one another, able to specifically bind to the transmembrane receptor IGF2R, expressed on the fibrogenic effector cell.

It is preferred that for the binding molecule according to the invention, wherein the transmembrane receptor is an IGF2R, the binding molecule is a multiparatopic binding molecule, preferably a biparatopic binding molecule. Preferably, binding of the first VHH comprised by the binding molecule to IGF2R enhances binding of the second VHH comprised by the binding molecule to IGF2R and preferably vice versa, and/or preferably wherein binding of the first VHH and the second VHH to IGF2R enhances internalization of the binding molecule compared to internalization of a second binding molecule comprising either the first VHH or the second VHH only.

An embodiment is the binding molecule according to the invention, wherein the binding molecule is a multiparatopic binding molecule, preferably a biparatopic binding molecule, wherein binding of the first VHH to IGF2R expressed on a cell of a first species and to IGF2R expressed on a cell of a second species enhances binding of the second VHH to IGF2R expressed on said cell of the first species and to IGF2R expressed on said cell of the second species, and preferably vice versa.

An embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB.

An embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule comprises at least two single variable antibody domains, preferably two single variable antibody domains, that are, independently from one another, able to specifically bind to the transmembrane receptor PDGFRB, expressed on the fibrogenic effector cell.

An embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule is a multiparatopic binding molecule, preferably a biparatopic binding molecule, preferably wherein binding of the first VHH comprised by the binding molecule to PDGFRB enhances binding of the second VHH comprised by the binding molecule to PDGFRB and preferably vice versa, and/or preferably wherein binding of the first VHH and the second VHH to PDGFRB enhances internalization of the binding molecule compared to internalization of a second binding molecule comprising either the first VHH or the second VHH only.

An embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule is a multiparatopic binding molecule, preferably a biparatopic binding molecule, wherein binding of the first VHH to PDGFRB expressed on a cell of a first species and to PDGFRB expressed on a cell of a second species enhances binding of the second VHH to PDGFRB expressed on said cell of the first species and to PDGFRB expressed on said cell of the second species, and preferably vice versa.

A further description of the VHHs, including humanization of VHHs, as well as other modifications, parts or fragments, derivatives or “VHH fusions”, multivalent constructs (including some non-limiting examples of linker sequences) and different modifications to increase the serum half-life of the VHHs and their preparations can be found e.g. in WO 08/101985 and WO 08/142164. One example is, e.g., the presence of a binding domain with affinity for human serum albumin.

Hence, in order to increase the half-life of the binding molecules according to the present invention, said binding molecules preferably comprise one or more other groups, residues, moieties or binding units that increase said half-life compared to a binding molecule without such groups, residues, moieties or binding units. These groups, residues, moieties or binding units are also referred to as half-life extenders.

The half-life extenders to be used in the binding molecules according to the present invention preferably increases the serum half-life of the binding molecules in humans with at least 1 hour, preferably at least 2 hours or longer. They may increase the half-life for more than 6 hours, such as more than 12 hours or even more than 24, 48 or 72 hours.

A particularly preferred binding unit to be used in the binding molecules according to the present invention is an albumin binding domain, an albumin binding VHH or an antibody Fc tail or fragment thereof.

An embodiment is the binding molecule according to the invention comprising at least two single variable antibody domains, wherein the at least two single variable antibody domains are separated by a linker amino acid sequence. The binding molecule either comprises a first VHH able to specifically bind to IGF2R and comprises a second VHH able to specifically bind to IGF2R, or comprises a first VHH able to specifically bind to PDGFRB and comprises a second VHH able to specifically bind to PDGFRB.

An embodiment is the binding molecule according to the invention, wherein the binding molecule comprises an N-terminal or a C-terminal cysteine or histidine residue, preferably an N-terminal or a C-terminal cysteine residue.

An embodiment is the binding molecule according to the invention, wherein the binding molecule comprises a half-life extender.

An embodiment is the binding molecule according to the invention, wherein the binding molecule comprises a half-life extender, wherein the half-life extender comprises or consists of one or more groups, residues, moieties or binding units that provide the binding molecule with increased half-life compared to a binding molecule without a half-life extender.

An embodiment is the binding molecule according to the invention, wherein the binding molecule comprises a half-life extender, wherein the half-life extender comprises or consists of one or more groups, residues, moieties or binding units that provide the binding molecule with increased half-life compared to a binding molecule without a half-life extender, wherein the groups, residues, moieties or binding units that provide the binding molecule with increased half-life are an albumin binding domain or an albumin binding VHH or a fragment thereof.

For the term “dAb” or “sdAb”, reference is e.g. made to Ward et al. 1989 (Nature 341 (6242): 544-6), to Holt et al. 2003 (Trends Biotechnol. 21 (11): 484-490) as well as to e.g. WO 04/068820, WO 06/030220, WO 06/003388.

It should also be noted that, although less preferred in the context of the present invention because they are not of mammalian origin, single variable domains can also be derived from certain species of shark (e.g., the so-called “IgNAR domains”, see e.g. WO 05/18629) as well as from mice (e.g. the so-called humabodies of Crescendo Biologics). The amino acid sequence and structure of an immunoglobulin sequence, in particular an immunoglobulin single variable domain, can be considered-without, however, being limited thereto—to be comprised of four framework regions or “FRs”, which are referred to in the art and herein as “Framework region 1” or “FR1”, as “Framework region 2” or “FR2”, as “Framework region 3” or “FR3”, and as “Framework region 4” or “FR4”, respectively. These framework regions are interrupted by three complementary determining regions or “CDRs”, which are referred to in the art as “Complementarity Determining Region 1” or “CDR1”, as “Complementarity Determining Region 2” or “CDR2”, and as “Complementarity Determining Region 3” or “CDR3”, respectively. The total number of amino acid residues in an immunoglobulin single variable domain can be in the region of 110-120, is preferably 112-115, and is most preferably 113. It should, however, be noted that parts, fragments, analogues or derivatives of an immunoglobulin single variable domain are not particularly limited as to their length and/or size, as long as such parts, fragments, analogues or derivatives meet the further requirements outlined herein and are also preferably suitable for the purposes described herein. Thus, in the meaning of the present invention, the term “immunoglobulin single variable domain” or “single variable antibody domain” comprises peptides which are derived from a non-human source, preferably from a camelid, preferably as a camel heavy chain antibody. They may be humanized, as previously described, e.g. in WO 08/101985 and WO 08/142164. Moreover, the term comprises binding molecules derived from non-camelid sources, e.g. mouse or human, which have been “camelized”, as previously described, e.g. in WO 08/101985 and WO 08/142164. The term “immunoglobulin single variable domain” encompasses immunoglobulin sequences of different origin, comprising mouse, rat, rabbit, donkey, human, and camelid immunoglobulin sequences. It also includes fully human, humanized or chimeric immunoglobulin sequences. E.g., it comprises camelid immunoglobulin sequences and humanized camelid immunoglobulin sequences or camelized immunoglobulin single variable domains, e.g. camelized dAb as described by Ward et al. (see e.g. WO 94/04678 and Davies and Riechmann 1994, Febs Lett. 339:285 and 1996, Protein Engineering 9:531).

As already said before, the at least one binding molecule can specifically bind to an extracellular domain of a receptor expressed on a fibrogenic effector cell. Preferably, the second single variable antibody domain, if present, is also able to specifically bind to a transmembrane receptor expressed on a fibrogenic effector cell. Particularly preferred is a binding molecule according to the invention comprising at least two single variable antibody domains capable of specifically binding to PDGFRB or comprising at least two single variable antibody domains capable of specifically binding to IGF2R.

Less preferred is a binding molecule according to the invention, comprising at least one single variable antibody domain capable of specifically binding to PDGFRB and at least one single variable antibody domain capable of specifically binding to IGF2R.

In one preferred embodiment, a binding molecule according to invention is provided, wherein binding of one of the single variable antibody domains to its antigen modulates the binding of the other single variable antibody domain to its antigen, preferably resulting in cluster induced endocytosis and fast receptor internalization.

The invention provides in a working example several VHHs that show excellent binding to and internalisation of the PDGFRB. A preferred embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule is a biparatopic binding molecule, wherein at least one, preferably both, of the first VHH and the second VHH is able to compete for binding to PDGFRB with any one or more of single domain antibodies having the sequence of SP01P with SEQ ID NO: 1, SP02P with SEQ ID NO: 9, SP05P with SEQ ID NO: 25, SP07P with SEQ ID NO: 33, SP08P with SEQ ID NO: 41, SP10P with SEQ ID NO: 49, SP12P with SEQ ID NO: 65, SP13P with SEQ ID NO: 73, SP14P with SEQ ID NO: 81, SP20P with SEQ ID NO: 113, SP26P with SEQ ID NO: 153 or SP28P with SEQ ID NO: 169. Preferably, the first VHH and the second VHH of the binding molecule do not both compete with the same single domain antibody having the sequence of SP01P, SP02P, SP05P, SP07P, SP08P, SP10P, SP12P, SP13P, SP14P, SP20P, SP26P or SP28P. In an embodiment, a binding molecule according to the invention is provided, comprising at least one single variable antibody domain that is able to compete with a single domain antibody having a sequence that is selected from any one of the sequences of SP02P or SP05P or SP14P or SP12P, depicted in Table 1 as SEQ ID Nos: 9, 25, 81, and 65, respectively, in specific binding to the PDGFRB. In an embodiment, the binding molecule comprises at least a second single variable antibody domain that is able to compete with a single domain antibody having one of the sequences of SP02P or SP05P or SP14P or SP12P, wherein preferably the first and second single variable antibody domain do not both compete with the same single domain antibody having one of the sequence of SP02P or SP05P or SP14P or SP12P. Alternatively and also preferred, the binding molecule comprises at least two single variable antibody domains wherein preferably the first and second single variable antibody domain both compete with the same single domain antibody having one of the sequences of SP02P or SP05P or SP14P or SP12P.

With the term “compete” is meant that in a competition assay, such as for instance described in Example 6 of the present invention, the addition of a binding molecule induces a significant decrease in binding of a given VHH, i.e., the VHH to which the competing is determined. “Significant” in this respect is preferably a decrease of >5%, preferably >10% when 250 nM of a binding molecule is added to 10 nM of a fluorescence labelled VHH in an ELISA. Example 8, e.g., shows a less than 5% less fluorescence intensity when 10 nM of labelled 13F11 and 250 nM of unlabelled 13E8 are allowed to bind to human IGF2R ectodomain in an ELISA, whereas substantially more decrease in fluorescence intensity is seen when 10 nM of labelled 13F11 and 250 nM of unlabelled 13A8, 13C11, 13G10, or 13A12 compete for the same or overlapping epitopes. From these data it is concluded that 13F11 and 13E8 do not compete with each other, whereas 13A8, 13C11, 13G10 and 13A12 compete with 13F11 in respect to binding to IGF2R. The same test conditions and threshold apply to a binding molecule directed to PDGFRB when determining whether a binding molecule competes with a given VHH.

TABLE 1
Amino acid sequence of single domain antibodies targeting PDGFRB. The estimated CDR
regions are underlined.

| Name | Amino acid sequence | SEQ ID NO: |
|---|---|---|
| SP01P | | 1 (whole VHH) |
| | EVQLVESGGGLAQAGGSLRLSCVAS | 2 (FR1) |
| | GNIDSANG | 3 (CDR1) |
| | MAWYRQAPGKQRELVAH | 4 (FR2) |
| | ITSGTS | 5 (CDR2) |
| | YYVASVEGRFTISRANAKDTWYLQMNSLKPEDTGVYYC | 6 (FR3) |
| | FPIGLSAHWSQ | 7 (CDR3) |
| | GTQVTVSS | 8 (FR4) |
| SP02P | | 9 (whole VHH) |
| | EVQLVESGGGLVQAGGSLRLSCAAS | 10 (FR1) |
| | ESISSIEQ | 11 (CDR1) |
| | MGWFRQAPGKQRALVAI | 12 (FR2) |
| | NTYGGRT | 13 (CDR2) |
| | VYTNSVEGRFTMSRDSAKNMVYLQMTSLEPEDTAVYYC | 14 (FR3) |
| | YAQTTAWRGGV | 15 (CDR3) |
| | WGQGTQVTVSS | 16 (FR4) |
| SP04P | | 17 (whole VHH) |
| | EVQLVESGGGLVQAGGSARLSCAAS | 18 (FR1) |
| | GSILSPNL | 19 (CDR1) |
| | MAWSRQAPGKQREVVAL | 20 (FR2) |
| | ATSDGIT | 21 (CDR2) |
| | TYATSVKGRFTISRDDAKNTVYLQMNSLKPEDTNLYTC | 22 (FR3) |
| | KYRALRAGAVDY | 23 (CDR3) |
| | WGQGTQVTVSS | 24 (FR4) |
| SP05P | | 25 (whole VHH) |
| | EVQLVESGGGLVQAGGSLRLSCAAS | 26 (FR1) |
| | GRTSFSGYA | 27 (CDR1) |
| | MAWFRQAPGKERDFVAS | 28 (FR2) |
| | ITWSYGYT | 29 (CDR2) |
| | YYTDSAQGRFTISRDSAKNTVYLEMNSLKPEDTAVYYC | 30 (FR3) |
| | AADPKASRFRILREYAY | 31 (CDR3) |
| | WGQGTQVTVSS | 32 (FR4) |
| SP07P | | 33 (whole VHH) |
| | EVQLVESGGGSVQTGGSLRLSCAAS | 34 (FR1) |
| | GRSFSTYA | 35 (CDR1) |
| | MAWFRQAPGKEREFVAA | 36 (FR2) |
| | INRRATDT | 37 (CDR2) |
| | VYADSAKGRFIISRDNDKNTVYLQMDSLKTEDTAVYYC | 38 (FR3) |
| | AAAKNAYDWRWDRLRDRDY | 39 (CDR3) |
| | WGQRGQGTQVTVSS | 40 (FR4) |
| SP08P | | 41 (whole VHH) |
| | EVQLVESGGGLVQAGGSLRLSCAAS | 42 (FR1) |
| | GRTFNGYA | 43 (CDR1) |
| | MAWFRQAPGKERDFVAS | 44 (FR2) |
| | ITWSYGYT | 45 (CDR2) |
| | YYADSAQGRFTISRDSAKNTVYLEMNSLKPEDTAVYYC | 46 (FR3) |
| | AADPKASRFRILRQYAH | 47 (CDR3) |
| | WGQGTQVTVSS | 48 (FR4) |
| SP10P | | 49 (whole VHH) |
| | EVQLVESGGGLVQAGGSLRLSCAAS | 50 (FR1) |
| | GRTFNGYA | 51 (CDR1) |
| | MGWFRQAPGKERDFVAA | 52 (FR2) |
| | ITWSYGYT | 53 (CDR2) |
| | HYAESVKGRFSISRDSANNMVYLEMNSLKPEDTAVYYC | 54 (FR3) |
| | AADPKASRFRTLRRYDY | 55 (CDR3) |
| | WGQGTQVTVSS | 56 (FR4) |
| SP11P | | 57 (whole VHH) |
| | EVQLVESGGGLVQAGDSLRLSCAAS | 58 (FR1) |
| | GRTFSSYP | 59 (CDR1) |
| | IAWFRQAPGKEREFVAA | 60 (FR2) |
| | ITSSG | 61 (CDR2) |
| | LTTYYANVVKGRFAISRDNAKDTVYLQMNSLKPEDTAVHYC | 62 (FR3) |
| | ATSNGFLSGRDIYQHNKYIY | 63 (CDR3) |
| | WGQGTQVTVSS | 64 (FR4) |
| SP12P | | 65 (whole VHH) |
| | EVQLVESGGGLVQAGGSLRLSCVIS | 66 (FR1) |
| | GRTSPTYP | 67 (CDR1) |
| | MGWFRQSPGNEREFVAS | 68 (FR2) |
| | INWSGGWR | 69 (CDR2) |
| | NYADSVKGRFTISRDDAKNTVYLQMNSLKPEDTAVYYC | 70 (FR3) |
| | ARGQYSSGTPVYAHEYAY | 71 (CDR3) |
| | WGQGTQVTVSS | 72 (FR4) |
| SP13P | | 73 (whole VHH) |
| | EVQLVESGGGLVQPGGSLRLSCAAS | 74 (FR1) |
| | ESIFSINY | 75 (CDR1) |
| | MAWYRQAPGKQRELVAF | 76 (FR2) |
| | SIDGSST | 77 (CDR2) |
| | NYVDSVRGRFTASRDNAENTLYLQMNSLKPEDTAVYYC | 78 (FR3) |
| | YAQGNTWAAGV | 79 (CDR3) |
| | WGQGTQVTVSS | 80 (FR4) |
| SP14P | | 81 (whole VHH) |
| | EVQLVESGGGLVQAGDSLRLSCVVS | 82 (FR1) |
| | GLTFSRYG | 83 (CDR1) |
| | MGWFRQATGKEREFVG | 84 (FR2) |
| | GISVGSSGT | 85 (CDR2) |
| | MYPNSVKGRFTISRDNAKSTVYLQMNSLKPEDTAVYFC | 86 (FR3) |
| | AAIDQGSFVQQRDYRY | 87 (CDR3) |
| | WGQGTQVTVSS | 88 (FR4) |
| SP15P | | 89 (whole VHH) |
| | EVQLVESGGGLVQAGGSLRLSCAAS | 90 (FR1) |
| | GRAFGGPY | 91 (CDR1) |
| | MAWFRQTPGKGREFVAA | 92 (FR2) |
| | ISPSSVYT | 93 (CDR2) |
| | FYQDSVKGRFTISRDNTKNTAYLQMNSLKPEDTAVYYC | 94 (FR3) |
| | AAEAAGEVRLETSYKY | 95 (CDR3) |
| | WGQGTQVTVSS | 96 (FR4) |
| SP17P | | 97 (whole VHH) |
| | EVQLVESGGGLVQPGGSLRLSCAAS | 98 (FR1) |
| | GLTFVNYA | 99 (CDR1) |
| | MGWFRQAPGKERELVAG | 100 (FR2) |
| | IASSGRI | 101 (CDR2) |
| | YYADSVAGRFTISRDNARNTVNLQMNSMKPEDTAVYYC | 102 (FR3) |
| | AGRRSFSSTSAADYNY | 103 (CDR3) |
| | WGQGTQVTVSS | 104 (FR4) |
| SP19P | | 105 (whole VHH) |
| | EVQLVESGGGLLQAGGSLTLSCAAS | 106 (FR1) |
| | GRTFNT | 107 (CDR1) |
| | MGWFRQASWKEREFVAS | 108 (FR2) |
| | IAWVGGSV | 109 (CDR2) |
| | FKSDSTKGRFTVSGDNAKNTVRLQMNSLKPEDTAVYYC | 110 (FR3) |
| | AARSGGTFDV | 111 (CDR3) |
| | WGQGTQVTVSS | 112 (FR4) |
| SP20P | | 113 (whole VHH) |
| | EVQLVESGGGLVQAGGSLRLSCVIS | 114 (FR1) |
| | GRTSPTYP | 115 (CDR1) |
| | MGWFRQSPGNEREFVAS | 116 (FR2) |
| | INWSGGWR | 117 (CDR2) |
| | NYADSVKDRFTISRDDAKNTVYLQMNSLKPEDTAVYYC | 118 (FR3) |
| | ARGQYSSGTPVYAHEYAY | 119 (CDR3) |
| | WGQGTQVTVSS | 120 (FR4) |
| SP21P | | 121 (whole VHH) |
| | EVQLVESGGGLVQPGGSLRLSCVVS | 122 (FR1) |
| | GSSFSSYT | 123 (CDR1) |
| | MGWFRRAPGKQRELVAG | 124 (FR2) |
| | VSTVGDT | 125 (CDR2) |
| | AAADSVKGRFTISRDNAKNTVYLQMNSLKPEDTAVYYC | 126 (FR3) |
| | NTYPHAYLE | 127 (CDR3) |
| | WGQGTQVTVSS | 128 (FR4) |
| SP22P | | 129 (whole VHH) |
| | EVQLVESGGGLVQAGGSLTLSCAAS | 130 (FR1) |
| | GRTFNT | 131 (CDR1) |
| | MGWFRQAPGKEREFVAS | 132 (FR2) |
| | IAWVGGSV | 133 (CDR2) |
| | FKSDSTKGRFTVSGDNAKNTVRLQMNSLKPEDTAVYYC | 134 (FR3) |
| | AARSGGTFDV | 135 (CDR3) |
| | WGQGTQVTVSS | 136 (FR4) |
| SP23P | | 137 (whole VHH) |
| | EVQLVESGGELVQAGGSLRLSCAVS | 138 (FR1) |
| | GRTLTSYP | 139 (CDR1) |
| | MGWFRQAPGKEREFVAA | 140 (FR2) |
| | ISWSGGDT | 141 (CDR2) |
| | MYADSVKGRFTISRDNSKNTVYLQMNSLKPEDTADYYC | 142 (FR3) |
| | ARRRYSSGISRHLVDYEY | 143 (CDR3) |
| | WGQGTQVTVSS | 144 (FR4) |
| SP25P | | 145 (whole VHH) |
| | EVQLVESGGGLVQPGGSLRLSCAAS | 146 (FR1) |
| | ESIFSINA | 14 (CDR1) |
| | MGWYRQTPGKQREMVAS | 148 (FR2) |
| | ITPGGFT | 149 (CDR2) |
| | IYADSVKGRFTISRDNAKNTLYLQMNNLRFEDTAVYYC | 150 (FR3) |
| | NAFAGSATSYHDFGS | 151 (CDR3) |
| | WGQGTQVTVSS | 152 (FR4) |
| SP26P | | 153 (whole VHH) |
| | EVQLVESGGGLVQAGGSLRLSCAAS | 154 (FR1) |
| | GRSFSSSV | 155 (CDR1) |
| | MGWFRQTPGKEREFVAA | 156 (FR2) |
| | TRWTAAST | 157 (CDR2) |
| | HYADSVKGRFTISRDHAENAVFLQMNSLKPEDTAVYYC | 158 (FR3) |
| | AAGNYLDTAQYRYNY | 159 (CDR3) |
| | WGQGTQVTVSS | 160 (FR4) |
| SP27P | | 161 (whole VHH) |
| | EVQLVESGGGLVQIGDSLRLSCAAS | 162 (FR1) |
| | GGTFNRYG | 163 (CDR1) |
| | MGWFRQAPGKEREFVAA | 164 (FR2) |
| | IRWDGVDT | 165 (CDR2) |
| | NYADFVKGRFTISRYNAKNTAYLQMNSLKPEDTAVYYC | 166 (FR3) |
| | AADRRGLYTKHAHRYDY | 167 (CDR3) |
| | WGQGTQVTVSS | 168 (FR4) |
| SP28P | | 169 (whole VHH) |
| | EVQLVESGGGLVQAGGSLRLSCAAS | 170 (FR1) |
| | GSIVSFNG | 171 (CDR1) |
| | INWYRQAPGKQREWVGG | 172 (FR2) |
| | ITQGGNT | 173 (CDR2) |
| | MYADSVRGRFTISRDNTKNTMYLQMNSLKPEDTAVYYC | 174 (FR3) |
| | RRPPVAS | 175 (CDR3) |
| | WGQGTQVTVSS | 176 (FR4) |
| SP29P | | 177 (whole VHH) |
| | EVQLVESGGGLVQAGDSLRLSCTVS | 178 (FR1) |
| | GRTFNTYV | 179 (CDR1) |
| | TGWFRQAPGKEREFVAA | 180 (FR2) |
| | IHQIGST | 181 (CDR2) |
| | YYRNSVKGRFTISRDGAKDTVYLQMNNLKPEDSAVYYC | 182 (FR3) |
| | AAGNGGYVMSDIAYGT | 183 (CDR3) |
| | WGQGTQVTVSS | 184 (FR4) |
| SP30P | | 185 (whole VHH) |
| | EVQLVESGGGLVQAGGSLRLSCAA | 186 (FR1) |
| | SRGTFGTDI | 187 (CDR1) |
| | MGWFRQAPGKEREFVAA | 188 (FR2) |
| | ISWRGANT | 189 (CDR2) |
| | YYGYSVKGRFTISRDNAKNTMYLQMADLKPEDTADYYC | 190 (FR3) |
| | GVHLNGTPYYYASGYRY | 191 (CDR3) |
| | WGQGTQVTVSS | 192 (FR4) |
| SP31P | | 193 (whole VHH) |
| | EVQLVESGGGLVQAGGSLRLSCAAS | 194 (FR1) |
| | RGTFGTYI | 195 (CDR1) |
| | MGWFRQAPGKEREFVAA | 196 (FR2) |
| | ISWRGANT | 197 (CDR2) |
| | YYGYSVKGRFTISRDNAKNTMYLQMADLKPEDTADYYC | 198 (FR3) |
| | GVHLNGTPYYYASGYRY | 199 (CDR3) |
| | WGQGTQVTVSS | 200 (FR4) |
| SP32P | | 201 (whole VHH) |
| | EVQLVESGGGTVQAGDSLRLSCTAS | 202 (FR1) |
| | GRTFSTYT | 203 (CDR1) |
| | MGWFRQAPGKERVVVAV | 204 (FR2) |
| | NTWNNFT | 205 (CDR2) |
| | VHQPSVKGRFTMSRDNTKNSIYLQMDSLKPEDTAVYYC | 206 (FR3) |
| | AASAKGTARYDY | 207 (CDR3) |
| | WGQGTQVTVSS | 208 (FR4) |
| SP33P | | 209 (whole VHH) |
| | EVQLVESGGGLVQAGGSLRLSCAAS | 210 (FR1) |
| | GSLRSINT | 211 (CDR1) |
| | MAWHRRAPGKEREWVAT | 212 (FR2) |
| | ITGSDET | 213 (CDR2) |
| | IVADSVKGRFAISRDAANNTLSLEMNGLKPEDTAVYYC | 214 (FR3) |
| | AAFTATLVPY | 215 (CDR3) |
| | WGQGTQVTVSS | 216 (FR4) |
| SP35P | | 217 (whole VHH) |
| | EVQLVESGGGLVQPGGSLRLSCAAS | 218 (FR1) |
| | GSLGSNNP | 219 (CDR1) |
| | MAWYRQAPGKERELVAS | 220 (FR2) |
| | ISSAYRT | 221 (CDR2) |
| | HYADFVKGRFTISRDSPKNTVSLQMNNLKPEDTAVYYC | 222 (FR3) |
| | GIFVSARNY | 223 (CDR3) |
| | WGKGTQVTVSS | 224 (FR4) |
| SP36P | | 225 (whole VHH) |
| | EVQLVESGGGLVQAGGSLRLSCTPS | 226 (FR1) |
| | ITTAGTTI | 227 (CDR1) |
| | MAWFRQAPGNEREFVAA | 228 (FR2) |
| | IWGSKTAYGD | 229 (CDR2) |
| | SMKGRLTISRDNVRDNGQKTVFLQMNNLQLQDTATYYC | 230 (FR3) |
| | AASSGGYVHSSTSYEI | 231 (CDR3) |
| | WGRGTQVTVSS | 232 (FR4) |

The invention further provides in a working example several VHHs that show excellent binding to and internalisation of the IGF2R. A preferred embodiment is the binding molecule according to the invention, wherein the transmembrane receptor is an IGF2R and wherein the binding molecule comprises a first VHH able to specifically bind to IGF2R and comprises a second VHH able to specifically bind to IGF2R, and wherein the binding molecule is a biparatopic binding molecule, wherein at least one, preferably both, of the first VHH and the second VHH is/are able to compete for binding to IGF2R with any one or more of single domain antibodies having the sequence of 13A8 with SEQ ID NO: 257, 13A12 with SEQ ID NO: 289, 13C11 with SEQ ID NO: 297, 13E8 with SEQ ID NO: 249, 13F11 with SEQ ID NO: 273 or 13G10 with SEQ ID NO: 305. Preferably, the first VHH and the second VHH do not both compete with the same single domain antibody having the sequence of 13A8, 13A12, 13C11, 13E8, 13F11 or 13G10. In an embodiment, a binding molecule according to the invention is provided, comprising at least one single variable antibody domain that is able to compete with a single domain antibody having the sequence of 13E8 or 13F11, depicted in Table 2 as SEQ ID Nos: 249 and 273, respectively, in specific binding to the IGF2R. In an embodiment, the binding molecule comprising at least a second single variable antibody domain that is able to compete with a single domain antibody having the sequence of 13E8 or 13F11, wherein preferably the first and second antibody do not both compete with the same single domain antibody having the sequence of 13E8 or 13F11. Alternatively and also preferred, the binding molecule comprises at least two single variable antibody domains wherein preferably the first and second single variable antibody domain both compete with the same single domain antibody having one of the sequences of 13E8 or 13F11.

TABLE 2
Amino acid sequences of the single domain antibodies targeting IGF2R are listed
in the table below. The estimated CDR regions are underlined.

| Name | Amino acid sequence | SEQ ID NO: |
|---|---|---|
| 13A2 | | 233 (whole VHH) |
| | EVQLVESGGGLVQAGGSLRLSCAASG | 234 (FR1) |
| | IIFSANTV | 235 (CDR1) |
| | GWYRQVPGKERDVVA | 236 (FR2) |
| | SITSGDSTYYG | 237 (CDR2) |
| | DSVRDRFTISRDNAKNTVYLQMNSLKPDDTAIYYC | 238 (FR3) |
| | RARTTDGSY | 239 (CDR3) |
| | WGQGTQVTVSS | 240 (FR4) |
| 13E5 | | 241 (whole VHH) |
| | EVQLVESGGGLVQAGGSLRLSCAASG | 242 (FR1) |
| | LTFSRYT | 243 (CDR1) |
| | MSWYRQAPGKEREVVA | 244 (FR2) |
| | AISSGDSTYHE | 245 (CDR2) |
| | DSVKGRFIISRDNAKNTVYLQMNSLKTEDTAVYYC | 246 (FR3) |
| | RANGPGTY | 247 (CDR3) |
| | WGQGTQVTVSS | 248 (FR4) |
| 13E8 | | 249 (whole VHH) |
| | EVQLVESGGGLVQAGGSLRLSCAAS | 250 (FR1) |
| | RSISP | 251 (CDR1) |
| | MGWYRQAPGKQRELVA | 252 (FR2) |
| | IMPSSGPPIYA | 253 (CDR2) |
| | DSVQGRFTISRDNAKNTVYLQMNSLKPEDTAVYYC | 254 (FR3) |
| | NVGGGY | 255 (CDR3) |
| | WGQGTQVTVSS | 256 (FR4) |
| 13A8 | | 257 (whole VHH) |
| | EVQLVESGGGLVPAGGSLRLSCAASG | 258 (FR1) |
| | RTFSNYA | 259 (CDR1) |
| | MGWFRQAPGKERKFVA | 260 (FR2) |
| | TISWSGGVTYY | 261 (CDR2) |
| | ADSVKGRFTISRDNAKNTVYLLMNSLKPEDTAVYYC | 262 (FR3) |
| | AAKRDSSSYDHRRYDY | 263 (CDR3) |
| | WGQGTQVTVSS | 264 (FR4) |
| 13A10 | | 265 (whole VHH) |
| | EVQLVESGGGLVQAGGSLRLSCSASG | 266 (FR1) |
| | SIFSINY | 267 (CDR1) |
| | MAWYRQEPGKEREVVA | 268 (FR2) |
| | SMSWRGDSTYL | 269 (CDR2) |
| | ADSVQGRFTISRNNAKNTMYLQMNSLKPEDTALYYC | 270 (FR3) |
| | KANNY | 271 (CDR3) |
| | WGQGTQVTVSS | 272 (FR4) |
| 13F11 | | 273 (whole VHH) |
| | EVQLVESGGRLVQPGDSLRLSCAASG | 274 (FR1) |
| | RTFSDNA | 275 (CDR1) |
| | MGWFRQAPGKERRFVA | 276 (FR2) |
| | GISWAGGSTYYS | 277 (CDR2) |
| | DSVKGRFTISRDNAKNTVYLQMNSLKPEDTAVYYC | 278 (FR3) |
| | AAGLRAWVQRMPKDYNY | 279 (CDR3) |
| | WGQGTQVTVSS | 280 (FR4) |
| 13G12 | | 281 (whole VHH) |
| | EVQLVESGGGLVQAGGSLRLSCAASG | 282 (FR1) |
| | RAFSISS | 283 (CDR1) |
| | MSWFRQAPGKERVVVA | 284 (FR2) |
| | SIAWSGDSTYYA | 285 (CDR2) |
| | DSVQGRFTISKDNAKNTLSLQMNSLKPEDTAVYYC | 286 (FR3) |
| | SAYTQVSVNNRY | 287 (CDR3) |
| | WGQGTQVTVSS | 288 (FR4) |
| 13A12 | | 289 (whole VHH) |
| | EVQLVESGGGLVQAGGSLRLSCAAS | 290 (FR1) |
| | GIIFGANT | 291 (CDR1) |
| | VGWYRQVPGKERDVVAS | 292 (FR2) |
| | ITSGDST | 293 (CDR2) |
| | YYGDSVRGRFTISRDNAKNTMYLQMNSLKPEDTAIYYC | 294 (FR3) |
| | RARTTDGSY | 295 (CDR3) |
| | WGQGTQVTVS | 296 (FR4) |
| 13C11 | | 297 (whole VHH) |
| | EVQLVESGGGLVQAGGSLRLSCAAS | 298 (FR1) |
| | GLTFSSYT | 299 (CDR1) |
| | MAWYRQAPGKEREVVAA | 300 (FR2) |
| | ISSGDSI | 301 (CDR2) |
| | YHEGSVQGRFIISRDNAKNTVYLQMNSLKAEDTAVYYC | 302 (FR3) |
| | RANGPGTY | 303 (CDR3) |
| | WGQGTQVTVSS | 304 (FR4) |
| 13G10 | | 305 (whole VHH) |
| | EVQLVESGGGLVQAGGSLRLSCAAS | 306 (FR1) |
| | GLTFSSYT | 307 (CDR1) |
| | MAWYRQAPGKEREVVAA | 308 (FR2) |
| | ISSGDSI | 309 (CDR2) |
| | YHEDSVQGRFIISRDNAKNTVYLQMNSLKAEDTAVYYC | 310 (FR3) |
| | RANGPGTY | 311 (CDR3) |
| | WGQGTQVTVSS | 312 (FR4) |
| 13A3 | | 313 (whole VHH) |
| | EVQLVESGGGLVQAGGSLRLSCAAS | 314 (FR1) |
| | GSIFRLNY | 315 (CDR1) |
| | MAWYRQAPGKEREVVA | 316 (FR2) |
| | SMSRRSDST | 317 (CDR2) |
| | YLADAVQGRFTISMNNAGNTMYLQMNSLKPEDTALYYC | 318 (FR3) |
| | KAND | 319 (CDR3) |
| | YWGQGTQVTVSS | 320 (FR4) |
| 13A10 | | 321 (whole VHH) |
| | EVQLVESGGGLVQAGGSLRLSCSAS | 322 (FR1) |
| | GSIFSINY | 323 (CDR1) |
| | MAWYRQEPGKEREVVA | 324 (FR2) |
| | SMSWRGDST | 325 (CDR2) |
| | YLADSVQGRFTISRNNAKNTMYLQMNSLKPEDTALYYC | 326 (FR3) |
| | KANNY | 327 (CDR3) |
| | WGQGTQVTVSS | 328 (FR4) |
| 13B7 | | 329 (whole VHH) |
| | EVQLVESGGGLVQAGGSLRLSCAAS | 330 (FR1) |
| | RSISP | 331 (CDR1) |
| | MGWYRQAPGKQRELVA | 332 (FR2) |
| | IMPSSGTPI | 333 (CDR2) |
| | YADSVQGRFTISRDNAKNTVYLQMNSLKPEDTAVYYC | 334 (FR3) |
| | NVGGG | 335 (CDR3) |
| | YWGQGTQVTVSS | 336 (FR4) |
| 13B12 | | 337 (whole VHH) |
| | EVQLVESGGGLVQAGGSLRLSCAAS | 338 (FR1) |
| | GIIFGANT | 339 (CDR1) |
| | VGWYRQVPGKERDVVA | 340 (FR2) |
| | SITSVDST | 341 (CDR2) |
| | YYGDSVRGRFTISRDNAKNTVYLQMNSLKPEDTAIYYC | 342 (FR3) |
| | RARTTDGS | 343 (CDR3) |
| | YWGQGTQVTVSS | 344 (FR4) |
| 13D11 | | 345 (whole VHH) |
| | EVQLVESGGGLVQAGGSLRLSCAAS | 346 (FR1) |
| | GSIFSINY | 347 (CDR1) |
| | MAWYRQAPGKEREVVA | 348 (FR2) |
| | SMSWRRDST | 349 (CDR2) |
| | YLADSVQGRFTISRNNAKNTMYLQMNSLKPEDTALYY | 350 (FR3) |
| | CKAND | 351 (CDR3) |
| | YWGQGTQVTVSS | 352 (FR4) |
| 13F4 | | 353 (whole VHH) |
| | EVQLVESGGGLVQAGGSLRLSCVAS | 354 (FR1) |
| | GSIFSINY | 355 (CDR1) |
| | MAWYRQTPGKEREVVA | 356 (FR2) |
| | SMSRRSDST | 357 (CDR2) |
| | YLADAVQGRFTISRNNAGNTMYLQMNSLKPEDTALYY | 358 (FR3) |
| | CKAND | 359 (CDR3) |
| | YWGQGTQVTVSS | 360 (FR4) |
| 13G12 | | 361 (whole VHH) |
| | EVQLVESGGGLVQAGGSLRLSCAASG | 362 (FR1) |
| | RAFSISS | 363 (CDR1) |
| | MSWFRQAPGKERVVVA | 364 (FR2) |
| | SIAWSGDSTYYA | 365 (CDR2) |
| | DSVQGRFTISKDNAKNTLSLQMNSLKPEDTAVYYC | 366 (FR3) |
| | SAYTQVSVNNRY | 367 (CDR3) |
| | WGQGTQVTVSS | 368 (FR4) |

A preferred embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule comprises the first VHH capable of specifically binding to PDGFRB, the first VHH comprising a CDR 1 sequence according SEQ ID NO: 155, a CDR 2 sequence according to SEQ ID NO: 157 and a CDR 3 sequence according to SEQ ID NO: 159, and comprises the second VHH capable of specifically binding to PDGFRB, the second VHH comprising any one of: a CDR 1 sequence according SEQ ID NO: 3, a CDR 2 sequence according to SEQ ID NO: 5 and a CDR 3 sequence according to SEQ ID NO: 7; or a CDR 1 sequence according SEQ ID NO: 11, a CDR 2 sequence according to SEQ ID NO: 13 and a CDR 3 sequence according to SEQ ID NO: 15; or a CDR 1 sequence according SEQ ID NO: 35, a CDR 2 sequence according to SEQ ID NO: 37 and a CDR 3 sequence according to SEQ ID NO: 39; or a CDR 1 sequence according SEQ ID NO: 75, a CDR 2 sequence according to SEQ ID NO: 77 and a CDR 3 sequence according to SEQ ID NO: 79; or any of the combinations of CDR1, CDR2 and CDR3 sequences wherein, independently, at most 4 amino acids, preferably at most 3, more preferably at most 2, most preferably at most 1, have/has been conservatively substituted, preferably independently selected from the following amino acid substitutions: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp or into Phe; and Val into Ile or into Leu, or any of the combinations of CDR1, CDR2 and CDR3 sequences wherein, independently, 85-99% of amino acids, preferably 90-99%, more preferably 95-99%, most preferably 97-99%, have been conservatively substituted.

A preferred embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule comprises the first VHH capable of specifically binding to PDGFRB, the first VHH comprising a CDR 1 sequence according SEQ ID NO: 155, a CDR 2 sequence according to SEQ ID NO: 157 and a CDR 3 sequence according to SEQ ID NO: 159, and comprises the second VHH capable of specifically binding to PDGFRB, the second VHH comprising a CDR 1 sequence according SEQ ID NO: 11, a CDR 2 sequence according to SEQ ID NO: 13 and a CDR 3 sequence according to SEQ ID NO: 15, or wherein, independently, at most 4 amino acids in one or two of the CDR 1 sequences, one or two of the CDR 2 sequences and/or one or two of the CDR 3 sequences, preferably at most 3, more preferably at most 2, most preferably at most 1, have/has been conservatively substituted, preferably independently selected from the following amino acid substitutions: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp or into Phe; and Val into Ile or into Leu, or wherein, independently, 85-99% of amino acids in one or two of the CDR 1 sequences, one or two of the CDR 2 sequences and/or one or two of the CDR 3 sequences, preferably 90-99%, more preferably 95-99%, most preferably 97-99%, have been conservatively substituted.

In an embodiment, a binding molecule according to the invention is provided, wherein the binding molecule comprises at least one single variable antibody domain capable of specifically binding to PDGFRB, the at least one single variable antibody domain comprising a CDR 1 sequence according SEQ ID NO: 83, a CDR 2 sequence according to SEQ ID NO: 85 and a CDR 3 sequence according to SEQ ID NO: 87; or a CDR 1 sequence according SEQ ID NO: 3, a CDR 2 sequence according to SEQ ID NO: 5 and a CDR 3 sequence according to SEQ ID NO: 7; or a CDR 1 sequence according SEQ ID NO: 11, a CDR 2 sequence according to SEQ ID NO: 13 and a CDR 3 sequence according to SEQ ID NO: 15; or a CDR 1 sequence according SEQ ID NO: 35, a CDR 2 sequence according to SEQ ID NO: 37 and a CDR 3 sequence according to SEQ ID NO: 39; or a CDR 1 sequence according SEQ ID NO: 75, a CDR 2 sequence according to SEQ ID NO: 77 and a CDR 3 sequence according to SEQ ID NO: 79; or a CDR 1 sequence according SEQ ID NO: 27, a CDR 2 sequence according to SEQ ID NO: 29 and a CDR 3 sequence according to SEQ ID NO: 31; or a CDR 1 sequence according SEQ ID NO: 43, a CDR 2 sequence according to SEQ ID NO: 45 and a CDR 3 sequence according to SEQ ID NO: 47; or a CDR 1 sequence according SEQ ID NO: 51, a CDR 2 sequence according to SEQ ID NO: 53 and a CDR 3 sequence according to SEQ ID NO: 55; or a CDR 1 sequence according SEQ ID NO: 115, a CDR 2 sequence according to SEQ ID NO: 117 and a CDR 3 sequence according to SEQ ID NO: 119; or a CDR 1 sequence according SEQ ID NO: 67, a CDR 2 sequence according to SEQ ID NO: 69 and a CDR 3 sequence according to SEQ ID NO: 71, or any of the combinations of CDR1, CDR2 and CDR3 sequences wherein, independently, at most 4 amino acids, more preferably at most 3, more preferably at most 2, most preferably at most 1, has been conservatively substituted, preferably independently selected from the following amino acid substitutions: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp or into Phe; and Val into Ile or into Leu.

An embodiment is the binding molecule according to the invention, wherein the transmembrane receptor is a PDGFRB, and wherein the binding molecule comprises a first VHH able to specifically bind to PDGFRB and comprises a second VHH able to specifically bind to PDGFRB, and wherein the binding molecule comprises the first VHH capable of specifically binding to PDGFRB, the first VHH having the sequence of SP26P with SEQ ID NO: 153, and comprises the second VHH capable of specifically binding to PDGFRB, the second VHH having the sequence of any one of: SP01P with SEQ ID NO: 1, SP02P with SEQ ID NO: 9, SP07P with SEQ ID NO: 33 and SP13P with SEQ ID NO: 73. Preferably, the binding molecule comprises the first VHH having the sequence of SP26P with SEQ ID NO: 153 and the second VHH having the sequence of SP02P with SEQ ID NO: 9.

An embodiment is the binding molecule according to the invention, wherein the transmembrane receptor is an IGF2R and wherein the binding molecule comprises a first VHH able to specifically bind to IGF2R and comprises a second VHH able to specifically bind to IGF2R, and wherein the binding molecule comprises the first VHH capable of specifically binding to IGF2R, the first VHH comprising a CDR 1 sequence according SEQ ID NO: 251, a CDR 2 sequence according to SEQ ID NO: 253 and a CDR 3 sequence according to SEQ ID NO: 255, and comprises the second VHH capable of specifically binding to IGF2R, the second VHH comprising any one of: a CDR 1 sequence according SEQ ID NO: 275, a CDR 2 sequence according to SEQ ID NO: 277 and a CDR 3 sequence according to SEQ ID NO: 279; or a CDR 1 sequence according SEQ ID NO: 259, a CDR 2 sequence according to SEQ ID NO: 261 and a CDR 3 sequence according to SEQ ID NO: 263; or a CDR 1 sequence according SEQ ID NO: 291, a CDR 2 sequence according to SEQ ID NO: 293 and a CDR 3 sequence according to SEQ ID NO: 295; or a CDR 1 sequence according SEQ ID NO: 299, a CDR 2 sequence according to SEQ ID NO: 301 and a CDR 3 sequence according to SEQ ID NO: 303; or a CDR 1 sequence according SEQ ID NO: 307, a CDR 2 sequence according to SEQ ID NO: 309 and a CDR 3 sequence according to SEQ ID NO: 311, or any of the combinations of CDR1, CDR2 and CDR3 sequences wherein, independently, at most 4 amino acids, preferably at most 3, more preferably at most 2, most preferably at most 1, have/has been conservatively substituted, or any of the combinations of CDR1, CDR2 and CDR3 sequences wherein, independently, 85-99% of amino acids, preferably 90-99%, more preferably 95-99%, most preferably 97-99%, have been conservatively substituted.

An embodiment is the binding molecule according to the invention, wherein the transmembrane receptor is an IGF2R and wherein the binding molecule comprises a first VHH able to specifically bind to IGF2R and comprises a second VHH able to specifically bind to IGF2R, wherein the binding molecule comprises the first VHH capable of specifically binding to IGF2R, the first VHH comprising a CDR 1 sequence according SEQ ID NO: 251, a CDR 2 sequence according to SEQ ID NO: 253 and a CDR 3 sequence according to SEQ ID NO: 255, and comprises the second VHH capable of specifically binding to IGF2R, the second VHH comprising a CDR 1 sequence according SEQ ID NO: 275, a CDR 2 sequence according to SEQ ID NO: 277 and a CDR 3 sequence according to SEQ ID NO: 279, or wherein, independently, at most 4 amino acids in one or two of the CDR 1 sequences, one or two of the CDR 2 sequences and/or one or two of the CDR 3 sequences, preferably at most 3, more preferably at most 2, most preferably at most 1, have/has been conservatively substituted, or wherein, independently, 85-99% of amino acids in one or two of the CDR 1 sequences, one or two of the CDR 2 sequences and/or one or two of the CDR 3 sequences, preferably 90-99%, more preferably 95-99%, most preferably 97-99%, have been conservatively substituted.

An embodiment is the binding molecule according to the invention comprising at least one single variable antibody domain capable of specifically binding to IGF2R, the at least one single variable antibody domain comprising a CDR 1 sequence according SEQ ID NO: 251, a CDR 2 sequence according to SEQ ID NO: 253 and a CDR 3 sequence according to SEQ ID NO: 255; or a CDR 1 sequence according SEQ ID NO: 275, a CDR 2 sequence according to SEQ ID NO: 277 and a CDR 3 sequence according to SEQ ID NO: 279; or a CDR 1 sequence according SEQ ID NO: 259, a CDR 2 sequence according to SEQ ID NO: 261 and a CDR 3 sequence according to SEQ ID NO: 263; or a CDR 1 sequence according SEQ ID NO: 291, a CDR 2 sequence according to SEQ ID NO: 293 and a CDR 3 sequence according to SEQ ID NO: 295; or a CDR 1 sequence according SEQ ID NO: 299, a CDR 2 sequence according to SEQ ID NO: 301 and a CDR 3 sequence according to SEQ ID NO: 303; or a CDR 1 sequence according SEQ ID NO: 307, a CDR 2 sequence according to SEQ ID NO: 309 and a CDR 3 sequence according to SEQ ID NO: 311, or any of the combinations of CDR1, CDR2 and CDR3 sequences wherein, independently, at most 4 amino acids, more preferably at most 3, more preferably at most 2, most preferably at most 1, has been conservatively substituted, preferably independently selected from the following amino acid substitutions: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp or into Phe; and Val into Ile or into Leu.

An embodiment is the binding molecule according to the invention comprising at least one single variable antibody domain capable of specifically binding to PDGFRB, wherein the at least one single variable antibody domain comprises or consists of any one of SEQ ID Nos: 81, 1, 9, 33, 73, 25, 41, 49, 65, or 113.

An embodiment is the binding molecule according to the invention, wherein the transmembrane receptor is an IGF2R and wherein the binding molecule comprises a first VHH able to specifically bind to IGF2R and comprises a second VHH able to specifically bind to IGF2R, and wherein the binding molecule comprises the first VHH capable of specifically binding to IGF2R, the first VHH having the sequence of 13E8 with SEQ ID NO: 249, and comprises the second VHH capable of specifically binding to IGF2R, the second VHH having the sequence of any one of: 13A8 with SEQ ID NO: 257, 13A12 with SEQ ID NO: 289, 13C11 with SEQ ID NO: 297, 13F11 with SEQ ID NO: 273 and 13G10 with SEQ ID NO: 305. Preferably, the binding molecule comprises the first VHH having the sequence of 13E8 with SEQ ID NO: 249 and the second VHH having the sequence of 13F11 with SEQ ID NO: 273.

An embodiment is the binding molecule according to the invention comprising at least one single variable antibody domain capable of specifically binding to IGF2R, wherein the at least one single variable antibody domain comprises or consists of any one of SEQ ID Nos: 249, 273, 257, 289, 297, or 305.

In an embodiment, a binding molecule according to the invention is provided, the binding molecule comprising at least 2 single variable antibody domains, wherein at least one single variable antibody domain comprises or consists of SEQ ID NOs: 81 and at least one single variable antibody domain comprises or consists of any one of SEQ ID NOs: 1, 9, 33, 73, 25, 41, 49, 65, or 113; or wherein at least one single variable antibody domain comprises or consists of any one of SEQ ID NOs: 1, 9, 33, or 73 and at least one single variable antibody domain comprises or consists of any one of SEQ ID NOs: 25, 41, 49, 65, or 113; or wherein at least one single variable antibody domain comprises or consists of any one of SEQ ID NOS: 25, 41, 49 and at least one single variable antibody domain comprises or consists of any one of SEQ ID NOs: 65 or 113. Such molecule includes a biparatopic anti-PDGFRB binding molecule.

An embodiment is the binding molecule according to the invention comprising at least 2 single variable antibody domains, wherein at least one single variable antibody domain comprises or consists of SEQ ID NOs: 249 and at least one single variable antibody domain comprises or consists of any one of SEQ ID NOs: 273, 257, 289, 297, or 305. Such molecule includes a biparatopic anti-IGF2R binding molecule.

An embodiment is the binding molecule according to the invention comprising at least 2 single variable antibody domains, wherein at least one single variable antibody domain comprises or consists of any one of SEQ ID NOs: 81, 1, 9, 33, 73, 25, 41, 49, 65, or 113 and at least one single variable antibody domain comprises or consists of any one of SEQ ID NOs: 249, 273, 257, 289, 297, or 305. Such binding molecule includes a bispecific binding molecule capable of binding to one epitope on the PDGFRB and one epitope on the IGF2R.

For the purposes of comparing two or more amino acid sequences, the percentage of “sequence identity” or “sequence similarity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence-compared to the first amino acid sequence—is considered as a difference at a single amino acid residue (position), i.e. as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the binding molecule. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as from WO 38/49185 and from the further references cited therein. Such conservative substitutions preferably are substitutions in which one amino acid within the following groups (a)-(e) is substituted by another amino acid residue within the same group: (a) small aliphatic, nonpolar or slightly polar residues: Ala, Val, Leu, Pro, Ile and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Ser, Thr, Sec and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp or into Phe; Val into Ile or into Leu.

Any amino acid substitutions applied to the binding molecules described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13:211, 1974 and Adv. Enzymol., 47:45-149, 1978, and on the analysis of hydrophobicity patterns in proteins developed by Eisenberg et al., Proc. Natl. Acad. Sci. USA 81:140-144, 1984; Kyte & Doolittle; J. Molec. Biol. 157:105-132, 1981, and Goldman et al., Ann. Rev. Biophys. Chem. 15:321-353, 1986, all incorporated herein in their entirety by reference.

The invention also encompasses optimized variants of these amino acid sequences. Generally, an “optimized variant” of an amino acid sequence according to the invention is a variant that comprises one or more beneficial substitutions such as substitutions increasing i) the degree of “humanization”, ii) the chemical stability, and/or iii) the level of expression.

In an embodiment, a binding molecule according to the invention comprising at least 2 single variable antibody domains is provided, wherein the at least two single variable antibody domains are separated by a linker amino acid sequence. In many instances, simple Gly-Ser linkers of 4-15 amino acids may suffice, but if greater flexibility of the amino acid chain is desired longer or more complex linkers may be used. Preferred linkers are (Gly₄Ser)n, (GSTSGS)n or any other linker that provides flexibility for protein folding. The binding domains may be separated only by a linker, but other useful amino acid sequences may be introduced between the binding domains or at the N-terminus or at the C-terminus of the first or last binding domain sequence, respectively. Thus, in one embodiment, a binding molecule according to the invention is provided, further comprising an amino acid sequence encoding a linker. Such linker sequence preferably provides flexibility within the molecule and increases the distance that can be bridged by the binding molecule in order to (cross) link two epitopes. Further, such linker may decrease steric hindrance that may occur when one of the single variable antibody domains has bound to its first target and the second single variable antibody domain must find and bind the second target.

It has been observed that a binding molecule according to the invention is internalized when a single variable antibody domain within the molecule binds its target with sufficient affinity. In a preferred embodiment, therefore, a binding molecule according to the invention is provided, wherein the at least one single variable antibody domain and, if present, additional single variable antibody domains are, independently from one another, capable of specifically binding to their respective receptor with a dissociation constant (K_{D}) of 10E-5 to 10E-12 moles/liter or less, and preferably of 10E-7 to 10E-12 moles/liter or less and more preferably of 10E-9 to 10E-12 moles/liter.

In the context of the present invention, “binding to and/or having affinity for” a certain antigen has the usual meaning in the art as understood e.g. in the context of antibodies and their respective antigens. In particular embodiments of the invention, the term “binds to and/or having affinity for” means that the at least one single variable antibody domain specifically interacts with an antigen. The term “specificity” refers to the number of different types of antigens or antigenic determinants to which a particular immunoglobulin sequence, antigen-binding moiety or antigen-binding molecule (such as a binding molecule of the invention) can bind. The specificity of an antigen-binding molecule can be determined based on affinity and/or avidity towards the target molecule(s) relative to affinity and/or avidity towards non-target molecules. The affinity, represented by the equilibrium constant for the dissociation of an antigen with an antigen-binding protein (K_{D}), is a measure for the binding strength between an antigenic determinant and an antigen-binding site on the antigen-binding protein: the lower the value of the K_{D}, the stronger the binding strength between an antigenic determinant and the antigen-binding molecule (alternatively, the affinity can also be expressed as the affinity constant (K_{A}), which is 1/K_{D}). Methods for determining the K_{D} will be clear to the skilled person, and for example include the techniques mentioned herein. The affinity denotes the strength or stability of a molecular interaction. The affinity is commonly given as by the K_{D}, or equilibrium dissociation constant, which has units of mol/liter (or M). The affinity can also be expressed as an association constant, K_{A}, which equals 1/K_{D} and has units of liter/mol. In the present specification, the stability of the interaction between two molecules (such as an amino acid sequence, immunoglobulin sequence, or binding molecule of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value.

The K_{D} value characterizes the strength of a molecular interaction also in a thermodynamic sense as it is related to the free energy of binding. As will be clear to the skilled person (for example on the basis of the further disclosure herein), affinity can be determined in a manner known per se, depending on the specific antigen of interest. Avidity gives a measure of the overall strength of an antibody-antigen complex. It is dependent on three major parameters: affinity of the antibody for the epitope (see above), valency of both the antibody and antigen, and structural arrangement of the parts that interact. Avidity is related to both the affinity between an antigenic determinant and its antigen binding site on the antigen-binding molecule and the number of pertinent binding sites present on the antigen-binding molecule. Typically, immunoglobulin sequences of the present invention (such as the amino acid sequences, ISVDs and/or binding molecules of the invention) will bind to their antigen with a K_{D} of 10E-5 to 10E-12 moles/liter or less, and preferably 10E-7 to 10E-12 moles/liter or less and more preferably 10E-9 to 10E-12 moles/liter. Any K_{D} value greater than 10E-5 M is generally considered to indicate non-specific binding. Preferably, a monovalent immunoglobulin sequence of the invention will bind to the desired antigen with an affinity less than 500 nM, preferably less than 200 nM, more preferably less than 10 nM, most preferably less than 500 pM. Binding specificity and binding affinity of an antigen binding protein or of an antigen-binding protein to an antigen or antigenic determinant can be determined in any suitable manner known per se, including, e.g., Scatchard analysis and/or competitive binding assays, such as radio-immunoassays (RIA), enzyme-linked immunoassays (ELISA) and sandwich competition assays, and the different variants thereof known per se in the art; as well as the other techniques mentioned herein. The K_{D} for biological interactions, such as the binding of a binding molecule of the invention to the cell associated antigen as defined herein, which are considered meaningful (e.g. specific), are typically in the range of 10E-10 M (0.1 nM) to 10E-6 M (1 HM).

The K_{D} can also be expressed as the ratio of the dissociation rate constant of a complex, denoted as k_{off}, to the rate of its association, denoted kₒₙ (so that K_{D}=K_{off}/kₒₙ and K_{A}=kₒₙ/k_{off}). The affinity of a molecular interaction between two molecules can be measured via different techniques known per se, such as the well-known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kₒₙ, K_{off} measurements and hence K_{D} (or K_{A}) values. This can for example be performed using the well-known Biacore instruments. Affinity measurements on transmembrane receptors that are expressed by cells are preferably performed by ELISA.

It will also be clear to the skilled person that the measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule. In such situation the measured affinity may be affected by the avidity of the interaction by the two molecules. Another approach that may be used to assess affinity is the 2-step ELISA procedure of Friguet et al. (J. Immunol. Methods, 77, 305-19, 1985). This method establishes a solution phase binding equilibrium measurement and avoids possible artefacts relating to adsorption of one of the molecules on a support such as plastic. However, the accurate measurement of K_{D} may be quite labour-intensive and as consequence, often apparent K_{D} values are determined to assess the binding strength of two molecules. It should be noted that as long as all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance.

A therapeutic or diagnostic molecule, if present, is also preferably bound to the remainder of the binding molecule by a linker and a spacer (i.e. a ‘linker-spacer’). Linkers are attached to elements of the proteinaceous targeting moiety, e.g. such as a binding molecule of the invention, such as lysine, histidine or cysteine residues. Spacers can be either cleavable or non-cleavable. The present invention provides conventional maleimide-based linker moieties but also a particular linker, further referred to as “Lx”, comprising a functional platinum (II) complex having two reactive groups such as, for instance but not limited to those described in international patent publication WO2013103301. Amongst other advantages, such as manufacturing advantages, Lx may confer superior stability to the binding molecules of the invention as compared to more conventional maleimide-based linkers (see Merkul et al, Expert Opin. Drug Deliv. 2019; 16:783-793). Hence, the linker and/or spacer preferably comprises or consists of a transition metal complex. In a preferred embodiment, the transition metal complex comprises Pt(II). In an even further preferred embodiment said linker comprises a cis-platinum (II) complex, more preferably a cis-platinum (II) complex comprising an inert bidentate moiety, wherein said bidentate moiety is preferably ethane-1,2-diamine.

As already mentioned before, reduction of the activation level of fibrogenic effector cells may attenuate fibrosis. In a preferred embodiment, therefore, a binding molecule according to the invention is provided, wherein the therapeutic molecule is able to inhibit the contractile, chemotactic, and/or pro-fibrotic ability of the fibrogenic effector cells, or wherein the therapeutic molecule is a cytotoxic molecule that may lead to the overall reduction of the numbers of fibrogenic effector cells by means of elimination.

An embodiment is the binding molecule according to the invention comprising at least two single variable antibody domains (VHH domains) and at least one therapeutic molecule, wherein the therapeutic molecule is able to inhibit any one or more of contractile activity, fibrogenic activity, chemotactic activity and pro-inflammatory activity of the effector cell. Preferably, the binding molecule comprises a single kind of therapeutic molecule, preferably covalently linked to a VHH domain. The binding molecule comprises at least one copy of the therapeutic molecule, such as 1-16 copies, preferably 1-8 copies, such as a single copy, two copies or four copies. Preferred is a single copy of the single kind of therapeutic molecule.

A preferred embodiment is the binding molecule according to the invention comprising at least two single variable antibody domains (VHH domains) and at least one therapeutic molecule, wherein the therapeutic molecule is a kinase inhibitor, preferably selected from Rho-kinase inhibitors, JAK-2 inhibitors or neprilysin inhibitors, or Angiotensin II Receptor antagonists, such as losartan, preferably wherein the therapeutic molecule is a Rho-kinase inhibitor or a JAK-2 inhibitor. Preferred therapeutic molecules conjugated to the VHH domains of the binding molecule are for example Y27632, pacritinib, sacubitril (at) and losartan. Y27632 is a ROCK (Rho-kinase) inhibitor. Pacritinib is a JAK-2 inhibitor, competing with JAK-2 for ATP binding, resulting in inhibition of JAK-2 activation, and inhibition of JAK-STAT signalling pathway. Sacubitril is a prodrug that is activated to sacubitrilat. Sacubitrilat is an inhibitor of neprilysin.

Kinase inhibitors, for example, can inhibit contraction of the cytoskeleton of activated myo-fibroblasts and activated pulmonary smooth muscle cells, which may be classified as fibrogenic effector cells. Examples of targeted kinases include kinases involved in a signalling pathway called the renin-angiotensin aldosterone system (RAAS) which plays a role in liver-, lung- and kidney fibrosis, and in portal and pulmonary hypertension (see e.g. Bargagli et al, Int. J. Mol. Sci., 2020; 21: E5663, Zhang et al, Adv. Exp. Med. Biol., 2019; 1165:671-691, and Tandon et al, J. Hepatol., 2010; 53:273-82). The RAAS plays a central role in the regulation of blood pressure by regulating vascular smooth muscle tone. Two intracellular kinases involved in RAAS are the tyrosine kinase Janus-kinase 2 (JAK2) and Rho associated coiled-coil containing protein kinase (ROCK). The expression of these protein kinases has been associated with several forms of human fibrosis as well as with portal- and pulmonary hypertension.

Within the RAAS, JAK2 becomes phosphorylated and activates Arhgef1, the nucleotide exchange factor responsible for activation of small GTPase RhoA, which in turn activates its downstream effector ROCK. ROCK is a regulator of the actomyosin cytoskeleton which promotes contractile force generation. ROCK phosphorylates and thereby inactivates myosin light chain phosphatase (MLCP), leading to increased myosin light chain phosphorylation and contraction. Therefore, use of a ROCK inhibitor, Y27632, would lead to a decrease in contraction induced by RAAS. In turn, because JAK2 plays a role in activating ROCK, its inhibition through SB1518 (also called pacritinib, which is a JAK2 inhibitor) can help to relax contracted fibrogenic effector cells. These small molecules are non-specific, thus they suffer dose-limiting toxicities rendering them unsuitable for systemic administration per se. A binding molecule of the invention bearing an efficacious amount of conjugated small-molecule drug is designed to overcome the limitations of the systemic administration of the small-molecule per se. Preferably, the therapeutic molecule is a kinase inhibitor, preferably selected from the group consisting of Rho-kinase, JAK-2- or neprilysin inhibitors, more preferably selected from the group consisting of Y27632, SB1518 and LBQ657.

Particularly preferred binding molecules of the invention are biparatopic binding molecules comprising either a first and a second VHH domain binding to IGF2R, or comprising a first and a second VHH domain binding to PDGFRB. Preferably, such binding molecule comprises a half-life extender, preferably an albumin binding domain (ABD), for example an albumin binding VHH. For a preferred binding molecule of the invention, the therapeutic molecule is a kinase inhibitor, preferably selected from Rho-kinase inhibitors, JAK-2 inhibitors or neprilysin inhibitors, or Angiotensin II Receptor antagonists, such as losartan. More preferably, for the binding molecule of the invention the therapeutic molecule is a Rho-kinase inhibitor or a JAK-2 inhibitor. According to the invention, typically suitable therapeutic molecules for conjugating to the VHH domains of the binding molecule are Y27632 which is a ROCK (Rho-kinase) inhibitor, and pacritinib which is a JAK-2 inhibitor. For the binding molecule of the invention comprising a therapeutic molecule, the ratio between the therapeutic molecule and the two VHH domains can be 1 to 1 or for example any ratio selected from 2:1 and 64:1, such as 3:1, 4:1, 5:1, 6:1, 7:1, 8:1, 10:1, 12:1, 16:1, 20:1, 24:1, 30:1, 32:1, 40:1, 50:1 and any ratio in between. That is to say, a binding molecule comprises a single copy of the therapeutic molecule, or a binding molecule comprises multiple copies of the therapeutic molecule, such as two, three, four, six, eight, ten, twelve, sixteen copies.

Preferred biparatopic binding molecules of the invention, comprising two VHH domains, are 13E8-13F11, 13F11-13E8, 13E8-13F11-ABD, 13F11-13E8-ABD, 13E8-13F11 (Y27632), 13E8-13F11-ABD (Y27632), 13E8-13F11 (pacritinib), 13E8-13F11-ABD (pacritinib), 13F11-13E8 (Y27632), 13F11-13E8-ABD (Y27632), 13F11-13E8 (pacritinib), 13F11-13E8-ABD (pacritinib), comprising optionally (and preferred) an albumin binding domain (ABD) and optionally (and preferred) a therapeutic molecule such as Y27632 or pacritinib as indicated. Preferred are 13E8-13F11-ABD (pacritinib), 13E8-13F11-ABD (Y27632), 13F11-13E8-ABD (Y27632) and 13F11-13E8-ABD (pacritinib), more preferred are 13F11-13E8 (Y27632), 13F11-13E8-ABD (Y27632), 13F11-13E8 (pacritinib) and 13F11-13E8-ABD (pacritinib). Particularly preferred is the binding molecule 13F11-13E8 (Y27632) or 13F11-13E8-ABD (Y27632).

Preferred biparatopic binding molecules of the invention, comprising two VHH domains, are SP02P-SP26P, SP26P-SP02P, SP02P-SP26P-ABD, SP26P-SP02P-ABD, SP02P-SP26P(Y27632), SP02P-SP26P-ABD (Y27632), SP02P-SP26P (pacritinib), SP02P-SP26P-ABD (pacritinib), SP26P-SP02P(Y27632), SP26P-SP02P-ABD (Y27632), SP26P-SP02P (pacritinib), SP26P-SP02P-ABD (pacritinib), comprising optionally (and preferred) an albumin binding domain (ABD) and optionally (and preferred) a therapeutic molecule such as Y27632 or pacritinib as indicated. Preferred are SP02P-SP26P-ABD (Y27632), SP02P-SP26P-ABD (pacritinib), SP26P-SP02P-ABD (Y27632) and SP26P-SP02P-ABD (pacritinib), more preferred are SP02P-SP26P(Y27632), SP02P-SP26P-ABD (Y27632), SP02P-SP26P (pacritinib) and SP02P-SP26P-ABD (pacritinib).

Surprisingly, the inventors provided relatively high-affinity biparatopic binding molecules comprising a first and a second VHH domain, wherein the high affinity was apparent despite of the respective lower binding affinities of the two corresponding monomeric VHHs to respective substrates (target receptor), demonstrating a synergistic contribution of each of the two monomeric VHHs to the binding characteristics of the resulting biparatopic constructs, and hence superiority of the biparatopic design over monomeric VHHs. In particular the synergy is apparent for binding molecules targeting the IGF2R, such as binding molecules comprising VHH domains 13E8 and 13F11.

In an embodiment, the therapeutic molecule is a cytotoxic molecule, such as a taxane, an anthracycline, a vinca alkaloid, a calicheamicin, a maytansinoid, an auristatin, preferably auristatin F, a tubulysin, a camptothecin, a pyrrolobenzodiazepine, a duocarmycin or an amanitin.

Normally, binding to a transmembrane receptor induces an intracellular signalling cascade. For instance, binding of PDGF to the PDGFRB induces phosphorylation of the receptor itself and other proteins, thereby engaging intracellular signalling pathways that trigger cellular responses such as migration and proliferation. As the purpose of a binding molecule of the present invention is the transportation of its payload into the cytosol of target cells and not activation of the receptor that is used for transportation, it is preferred that the binding of the binding molecule to the extracellular domain of the transmembrane receptor does not induce the receptor's intracellular signalling cascade. It is further preferred that binding of the binding molecule to the transmembrane receptor leads to receptor mediated internalisation, endocytosis and release of the drug or cytotoxic molecule within the endolysosomal compartment.

In one preferred embodiment, a binding molecule according to the invention is provided, wherein the binding molecule comprises an N-terminal or a C-terminal cysteine or histidine residue, preferably an N-terminal or C-terminal cysteine that serves the purpose of conjugation to the linker molecule. In embodiments, the invention provides a biparatopic binding molecule comprising at least two binding molecules according to the invention.

As mentioned previously, a binding molecule according to the invention, comprising a therapeutic molecule, serves to deliver the therapeutic molecule to the cytosol of a target cell, e.g., a fibrogenic effector cell, thereby enabling the treatment of a corresponding fibrotic affliction. The invention, therefore, provides an internalising binding molecule according to the invention for use as a medicament.

The term “treat”, “treating”, or “treatment” refers to administering a therapy in an amount, manner, and/or mode effective to improve a condition, symptom, or parameter associated with a disease or to prevent progression of a disease, to either a statistically significant degree or to a degree detectable to one skilled in the art. In the case of therapeutic use, the treatment may improve, cure, maintain, or decrease duration of the disease or condition in the subject. In therapeutic uses, the subject may have a partial or full manifestation of the symptoms. In a typical case, treatment improves the disease or condition of the subject to an extent detectable by a physician or prevents worsening of the disease or condition. As used herein, the term “prevent” or “preventing” means mitigating a symptom of the referenced disorder. In particular, said term encompasses the complete range of therapeutically positive effects of administrating a binding molecule of the invention to a subject including reduction of, alleviation of, and relief from a (secondary) complication of a fibrotic affliction, e.g. oesophageal variceal haemorrhage, and the symptoms thereof. The term “prevention” includes the prevention or postponement of development of the disease, prevention or postponement of development of complications and/or a reduction in the severity of such complications and their symptoms that will or are expected to develop. These further include ameliorating existing symptoms, preventing additional symptoms and ameliorating or preventing the underlying causes of symptoms. As used herein, the terms “subject” and “patient” are used interchangeably. As used herein, the terms “subject” and “subjects” refer to an animal, e.g., a mammal including a non-primate (e.g., a cow, pig, horse, donkey, goat, camel, cat, dog, guinea pig, rat, mouse, sheep) and a primate (e.g., a monkey, such as a cynomolgus monkey, gibbon, orangutan, gorilla, chimpanzee, and a human). A “patient” preferably refers to a human. Said patient can include elderly, adults, adolescents and children, from any age, for instance children ranging from the age of 2 years to less than 12 years, adolescents ranging from 12 years to less than 18 years, adults ranging from 18 years to less than 65 years, and elderly from 85 years and up.

An aspect of the invention relates to the binding molecule according to the invention for use as a medicament. Preferably, such medicament is for use in a method for the prevention and/or treatment of a disease or disorder associated with or characterized by the increased expression of PDGFRB and/or IGF2R.

Preferably, such medicament is for use in a method for the prevention and/or treatment of fibrotic afflictions, preferably liver fibrosis, lung fibrosis, kidney fibrosis, systemic sclerosis/scleroderma, inflammatory bowel disease, and certain sclerosing cancerous malignancies (sclerosing) malignant tumors.

Preferably, such medicament is for use in a method for the prevention and/or treatment of (secondary) clinical manifestations that are attributable to said fibrotic afflictions, such as portal- or pulmonary (arterial and/or venous) hypertension in the cases of liver fibrosis and/or cirrhosis, and pulmonary fibrosis, respectively.

Preferably, such medicament is for use in a method for the prevention and/or treatment of acute complications of such (secondary) clinical manifestations, such as oesophageal bleeding or cardiovascular or pulmonary failure in the cases of portal hypertension and pulmonary (arterial and/or venous) hypertension, respectively.

For instance, portal hypertension (PH) is present in liver fibrosis and in all stages of liver cirrhosis. In liver cirrhosis, the activated HSCs contract around the sinusoids they reside on. This leads to PH where blood flow is obstructed through the portal venous system in the liver. The portal venous system is made up of the portal vein which merges a large volume of blood coming from the stomach, intestine, spleen, and pancreas, branching them through smaller vessels travelling throughout the liver. If the vessels in the liver are blocked due to liver damage, blood cannot flow properly through the liver resulting in high blood pressure. If PH worsens and blood flow is obstructed through the portal vein, blood is redirected through smaller blood vessels such as those in the oesophagus that are not designed to carry such large volumes of blood. This leads to dilated veins (varices) at the distal oesophagus and/or the proximal stomach which have an increased risk of rupturing (variceal haemorrhages), causing severe to fatal internal bleeding. A binding molecule according to the invention is particularly useful for targeting the cells that cause or exacerbate liver cirrhosis, liver fibrosis and/or portal hypertension.

An aspect of the invention relates to the binding molecule according to the invention, for use in a method for the prevention and/or treatment of a disease or disorder associated with or characterized by the upregulation of PDGFRB and/or IGF2R expression.

An embodiment is the binding molecule for use in a method for the prevention and/or treatment of a disease or disorder associated with or characterized by the upregulation of PDGFRB and/or IGF2R expression according to the invention, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, preferably any one or more of: lung fibrosis, renal fibrosis, systemic sclerosis/scleroderma, inflammatory bowel disease and a cancer relating to a (sclerosing) malignant tumor such as breast cancer, lung cancer, colon cancer and prostate cancer.

An embodiment is the binding molecule for use in a method for the prevention and/or treatment of a disease or disorder associated with or characterized by the upregulation of PDGFRB and/or IGF2R expression according to the invention, wherein the prevention and/or the treatment is the prevention and/or the treatment of a (secondary) clinical manifestation that is attributable to liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.

An embodiment is the binding molecule for use in a method for the prevention and/or treatment of a disease or disorder associated with or characterized by the upregulation of PDGFRB and/or IGF2R expression of the invention, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.

An aspect of the invention relates to a nucleic acid that encodes amino acid residues of at least part of a binding molecule of the invention, and preferably the amino acid residues of the binding molecule according to the invention. That is to say, the nucleic acid preferably encodes the amino acid residues of the binding molecule according to the invention, wherein the transmembrane receptor is an IGF2R, and wherein the binding molecule comprises a first VHH able to specifically bind to IGF2R and comprises a second VHH able to specifically bind to IGF2R, or wherein the transmembrane receptor is a PDGFRB, and wherein the binding molecule comprises a first VHH able to specifically bind to PDGFRB and comprises a second VHH able to specifically bind to PDGFRB. In one embodiment, the invention provides a nucleic acid that encodes at least part of a binding molecule according to the invention. Although one of the advantages of the present invention is the ease of production and the simplicity of the molecules of the invention, the choice for a single nucleic acid encoding all necessary functions in itself enables the relatively easy addition (to the extent that there is room in the chosen expression vectors, etc.) of other functionalities in the resulting binding molecule. Such nucleic acid enables production of a binding molecule according to the invention when transfected in a suitable host cell. Therefore, the invention provides a host cell for expression of at least part of a binding molecule according to the invention, comprising a nucleic acid according to the invention. An aspect of the invention relates to a host cell for expression of amino acid residues of at least part of a binding molecule of the invention, and preferably the amino acid residues of the binding molecule according to the invention, comprising a nucleic acid according to the invention.

An aspect of the invention relates to a method for producing a binding molecule according to the invention, comprising culturing a host cell of the invention, allowing for expression of amino acid residues of at least part of said binding molecule, preferably the amino acid residues of the binding molecule of the invention, harvesting the expressed amino acid residues of the at least part of the binding molecule or of the binding molecule, and coupling the therapeutic molecule or the diagnostic molecule to said amino acid residues of part of said binding molecule or of the binding molecule, optionally through a linker. In one embodiment, the invention provides a method for producing a binding molecule according to the invention, comprising culturing a host cell according to the invention, allowing for expression of at least part of said binding molecule, harvesting the binding molecule, and coupling the therapeutic or diagnostic molecule to said part of said binding molecule, optionally through a linker as defined previously.

A binding molecule of the invention can be produced by any commonly used method. Typical examples include the recombinant expression in suitable host systems, e.g. bacteria or yeast or mammalian cells. The binding molecules of the invention will undergo a suitable purification regimen prior to being formulated in accordance to the present invention. In general, the binding molecules of the invention are produced by living host cells that have been genetically engineered to produce the binding molecule. Methods of genetically engineering cells to produce proteins are well known in the art. See e.g. Ausubel et al., eds. (1990), Current Protocols in Molecular Biology (Wiley, New York). Such methods include introducing nucleic acids that encode and allow expression of the binding molecule into living host cells. These host cells can be bacterial cells, fungal cells, or animal cells grown in culture. Bacterial host cells include, but are not limited to, Escherichia coli cells. Examples of suitable E. coli strains include: BL21 (D3), HB101, DH5a, GM2929, JM109, KW251, NM538, IMM539, and any E. coli strain that fails to cleave foreign DNA. Preferred is E. coli strain BL21 (D3). Fungal host cells that can be used include, but are not limited to, Saccharomyces cerevisiae, Pichia pastoris and Aspergillus cells. A few examples of animal cell lines that can be used are CHO, VERO, BHK, HeLa, Cos, MDCK, HEK293, 3T3, and WI38. New animal cell lines can be established using methods well known by those skilled in the art (e.g., by transformation, viral infection, and/or selection). Optionally, the binding molecule can be secreted by the host cells into the medium. In some embodiments, the binding molecules can be produced in bacterial cells, e.g., in E. coli cells.

In one embodiment, the binding molecules are expressed in a yeast cell such as Pichia (see, e.g., Powers et al., J. Immunol. Methods 251:123-35 (2001)), Hansenula, or Saccharomyces. In one embodiment, binding molecules are produced in mammalian cells. Typical mammalian host cells for expressing the clone antibodies or antigen-binding fragments thereof include Chinese Hamster Ovary (CHO cells) (including dhfr-CHO cells, described in Urlaub and Chasin, Proc. Natl. Acad. Sci. USA 77:4216-4220 (1980), used with a DHF selectable marker, e.g., as described in Kaufman and Sharp, Mol. Biol. 159:601-621 (1982)), lymphocytic cell lines, e.g., NS0 myeloma cells and SP2 cells, COS cells, and a cell from a transgenic animal, e.g., a transgenic mammal. For example, the cell is a mammary epithelial cell. In addition to the nucleic acid sequences encoding the binding molecule, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5, 179,017). E.g., typically the selectable marker gene confers resistance to drugs, such as G418, hygromycin, or methotrexate, on a host cell into which the vector has been introduced. Standard molecular biology techniques can be used to prepare the recombinant expression vector, transfect the host cells, select for transformants, culture the host cells and recover the antibody molecule from the culture medium. E.g., the binding molecules of the invention can be isolated by affinity chromatography. In one embodiment, the binding molecule of the invention is purified as described in WO 10/058550. In an exemplary embodiment, the binding molecule is purified from one or more contaminants by: contacting a mixture of binding molecule and contaminant(s) with a Protein A-based support and/or an ion exchange support, under conditions that allow the binding molecule to bind to or adsorb to the support; removing one or more contaminants by washing the bound support under conditions where the binding molecule remains bound to the support, and selectively eluting the binding molecule from the support by eluting the adsorbed binding molecule with an elution buffer. The binding molecules of the invention can also be produced by a transgenic animal. E.g., U.S. Pat. No. 5,849,992 describes a method of expressing an antibody in the mammary gland of a transgenic mammal. A transgene is constructed that includes a milk-specific promoter and nucleic acids encoding the antibody molecule and a signal sequence for secretion. The milk produced by females of such transgenic mammals includes, secreted therein, the single domain of interest. The antibody molecule can be purified from the milk, or for some applications, used directly. The present invention encompasses methods of producing the formulations as defined herein.

The invention further provides a pharmaceutical composition comprising at least one binding molecule according to the invention and at least one pharmaceutically acceptable excipient. An aspect of the invention relates to a pharmaceutical composition comprising at least one binding molecule according to the invention and at least one pharmaceutically acceptable excipient. Preferably, the pharmaceutical composition comprises one binding molecule of the invention, or two.

A binding molecule of the invention can be administered or used for administration in the form of a liquid solution (e.g., injectable and infusible solutions). Such compositions can be administered by a parenteral mode (e.g., subcutaneous, intraperitoneal or intramuscular injection) or by inhalation. The phrases “parenteral administration” and “administered parenterally” as used herein mean modes of administration other than enteral and topical administration, usually by injection, and include subcutaneous (s.c.) or intramuscular administration as well as intravenous (i.v.), intracapsular, intraorbital, intracardiac, intradermal, intraperitoneal, transtracheal, subcuticular, subcapsular, subarachnoid, intraspinal, epidural, and intrasternal injection and infusion. Preferably, the second or further doses of a binding molecule of the invention described herein are administered subcutaneously or orally, for slow release and, hence, a sustained effect. Preferably and in particular in acute situations, it is preferred to administer a binding molecule of the invention orally as the binding molecules will be transported via the splanchnic circulation directly towards the diseased liver after uptake in the gut.

The present invention provides also formulations of binding molecules comprising at least one immunoglobulin single variable domain against PDGFRB or IGF2R, which are stable, and preferably suitable for pharmaceutical uses, including the preparation of medicaments (also called “pharmaceutical composition of the invention”). The term “pharmaceutical composition” refers to a preparation which is in such form as to permit the biological activity of the active ingredient (the binding molecule of the invention) to be effective, and which contains no additional components which are unacceptably toxic to a subject to which the formulation would be administered. Such formulations are preferably sterile. “Pharmaceutically acceptable” excipients (vehicles, additives) are those which can reasonably be administered to a subject mammal to provide an effective dose of the active ingredient employed.

The term “excipient” as used herein refers to an inert substance which is commonly used as a diluent, vehicle, preservative, lyoprotectant, surfactant, binder, carrier or stabilizing agent for compounds which impart a beneficial physical property to a formulation. The skilled person is familiar with excipients suitable for pharmaceutical purposes, which may have particular functions in the formulation, such as lyoprotection, stabilization, preservation, etc.

Non-limiting examples of agents that can be co-formulated with a binding molecule of the invention include, e.g., adjunctive treatment (e.g. corticosteroids such as (methyl) prednisolone or (methyl) prednisone, diuretics, albumin, vitamin K, antibiotics and nutritional therapy and systemic blood pressure lowering agents such as beta-blocking agents) and supportive therapy with red cell transfusion. Such combination therapies may advantageously utilize lower dosages of the administered therapeutic agents, thus avoiding possible toxicities or complications associated with the various monotherapies. In an embodiment, the present invention relates to a combination therapy of a binding molecule of the invention together with a systemic blood pressure lowering treatment. Preferably, the combination therapy is provided until the portal pressure is normalized. Portal pressure measurement helps to confirm the diagnosis and monitor the course of the disease and a possible need for additional treatments. The efficacy of any particular proteinaceous molecule of the invention or dosing regimen may be determined by methods available to those of skill in the art. Briefly, during a clinical trial, the patients may be observed by medical personnel and the state of disease is assessed by any combination of criteria. The improvement of a patient's disease state is determined based on these criteria at numerous time points and the combination of these determinations on a patient population is plotted to assess the efficacy of treatment. In exemplary embodiments, assessment of efficacy may be measured by any or all of the criteria set forth below:
Time-to-response of treatment
Number of subjects with complete remission.
Number of (subjects with) exacerbations (e.g. oesophageal bleedings in portal hypertension) or time to first exacerbation of EVH.
Improvement of organ function and improvement of disease-related signs and symptoms.
Total mortality within study drug treatment period (including tapering).
Determination of biomarkers of disease, such as portal pressure, pulmonary arterial or pulmonary venous pressure

The dosage of a binding molecule according to the invention is to be established through animal studies and clinical studies in so-called rising-dose experiments. Typically, the doses will be comparable with present day antibody dosages (at the molar level, the weight of the invented molecules may differ from that of antibodies). Typically, such dosages are 3-15 mg/kg body weight or 25-1000 mg per dose.

Especially in the more chronic stages of said fibrotic afflictions, the first applications of a binding molecule according to the invention will (at least initially) probably take place in combination with other treatments (standard care). Thus, the invention also provides a pharmaceutical composition comprising a binding molecule according to the invention and a conventional therapy e.g. a blood pressure lowering drug such as propranolol or surgical means to stop bleedings from e.g. varices in the oesophagus. Moreover, the current invention also provides a pharmaceutical composition comprising a binding molecule according to the invention for use in an adjuvant treatment, e.g. in said fibrotic afflictions. Additionally, the current invention also provides a pharmaceutical composition comprising a binding molecule according to the invention for use in a combination medicinal treatment of said fibrotic afflictions.

Therefore, the ideal therapeutic compound should allow the delivery of effector compounds which are much more potent than established small molecule pharmacological compounds, and which otherwise have a therapeutic window which is too narrow to allow their use as free drug. Preferably, stable conjugation of the effector compound to the targeting moiety avoids systemic toxicity by release of the (otherwise intolerable) free compound in the circulation. Once the molecule is internalized by the fibrogenic effector cell, the drug should be released to be able to mediate deactivation of said cell.

An aspect of the invention relates to the pharmaceutical composition of the invention, for use as a medicament.

An aspect of the invention relates to the pharmaceutical composition of the invention, for use in a method for the prevention and/or treatment of a disease or disorder associated with or characterized by the upregulation of PDGFRB and/or IGF2R expression.

Preferably, the pharmaceutical composition further comprises at least one other compound useful in the treatment of said medical conditions. An aspect of the invention relates to a pharmaceutical composition comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor.

An aspect of the invention relates to the pharmaceutical composition of the invention comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor, for use as an adjuvant treatment of secondary hemodynamic manifestations of fibrotic disease, such as portal hypertension and pulmonary hypertension.

An aspect of the invention relates to the pharmaceutical composition of the invention comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor, for use as an adjuvant treatment of secondary hemodynamic manifestations of fibrotic disease, such as portal hypertension and pulmonary hypertension, wherein said use is in the treatment of an acute manifestation of: portal hypertension such as acute esophageal bleeding or pulmonary hypertension such as acute cardiac decompensation.

A pharmaceutical composition according to the invention is preferably for use as an adjuvant treatment of variceal bleeding in the presence of portal hypertension in liver fibrosis and cirrhosis and/or for use in the lowering of pulmonary arterial or venous pressure in (e.g. lung fibrosis with) pulmonary hypertension.

Thus, in summary:

An aspect of the invention relates to a binding molecule according to the invention, for use in a method for the prevention and/or treatment of a disease or disorder associated with or characterized by the upregulation of PDGFRB and/or IGF2R expression.

An embodiment is the pharmaceutical composition for use in a method for the prevention and/or treatment of a disease or disorder associated with or characterized by the upregulation of PDGFRB and/or IGF2R expression according to the invention, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, preferable any one or more of: lung fibrosis, renal fibrosis, systemic sclerosis/scleroderma, inflammatory bowel disease and a cancer relating to a (sclerosing) malignant tumor such as breast cancer, lung cancer, colon cancer and prostate cancer.

An embodiment is the pharmaceutical composition for use in a method for the prevention and/or treatment of a disease or disorder associated with or characterized by the upregulation of PDGFRB and/or IGF2R expression according to the invention, wherein the prevention and/or the treatment is the prevention and/or the treatment of a (secondary) clinical manifestation that is attributable to said liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.

An embodiment is the pharmaceutical composition for use of the invention, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.

An embodiment is the binding molecule for use according to the invention, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer or prostate cancer, preferably any one or more of: lung fibrosis, renal fibrosis, systemic sclerosis/scleroderma, inflammatory bowel disease and a cancer relating to a (sclerosing) malignant tumor such as breast cancer, lung cancer, colon cancer or prostate cancer.

An embodiment is the binding molecule of the invention for use according to the invention, wherein the prevention and/or the treatment is the prevention and/or the treatment of a (secondary) clinical manifestation that is attributable to said liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer or prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.

An embodiment is the binding molecule of the invention for use according to the invention, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.

An aspect of the invention relates to a pharmaceutical composition comprising at least one binding molecule according to the invention and at least one pharmaceutically acceptable excipient.

An aspect of the invention relates to the pharmaceutical composition of the invention, for use in a method for the prevention and/or treatment of a disease or disorder associated with or characterized by the upregulation of PDGFRB and/or IGF2R expression.

An embodiment is the pharmaceutical composition of the invention for use according to the invention, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer or prostate cancer, preferable any one or more of: lung fibrosis, renal fibrosis, systemic sclerosis/scleroderma, inflammatory bowel disease and a cancer relating to a (sclerosing) malignant tumor such as breast cancer, lung cancer, colon cancer or prostate cancer.

An embodiment is the pharmaceutical composition of the invention for use according to the invention, wherein the prevention and/or the treatment is the prevention and/or the treatment of a (secondary) clinical manifestation that is attributable to said liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer or prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.

An embodiment is the pharmaceutical composition of the invention for use according to the invention, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.

An aspect of the invention relates to a pharmaceutical composition comprising at least one binding molecule according to the invention and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor.

An embodiment is the pharmaceutical composition according to the invention, for use as an adjuvant treatment of secondary hemodynamic manifestations of fibrotic disease, such as portal hypertension and pulmonary hypertension.

An embodiment is the pharmaceutical composition according to the invention, for use in the treatment of an acute manifestation of: portal hypertension such as acute esophageal bleeding or pulmonary hypertension such as acute cardiac decompensation.

An embodiment is the binding molecule according to the invention, comprising a diagnostic molecule, wherein the diagnostic molecule is an imaging agent. A binding molecule according to the invention, comprising a diagnostic molecule is in particular useful for diagnosis of, said medical conditions. In a preferred embodiment, therefore, a binding molecule according to the invention comprising a diagnostic molecule is provided, wherein the diagnostic molecule is an imaging agent. Provided is a diagnostic composition comprising at least one binding molecule according to the invention comprising a diagnostic molecule and a diluent and/or excipient. An aspect of the invention relates to a diagnostic composition comprising at least one binding molecule of the invention comprising a diagnostic molecule, wherein the diagnostic molecule is an imaging agent, and comprising a diluent and/or excipient. An aspect of the invention relates to the use of the binding molecule of the invention comprising a diagnostic molecule, wherein the diagnostic molecule is an imaging agent, or to the use of the diagnostic composition of the invention, in a method for diagnosing of a disease or disorder associated with or characterized by the upregulation of PDGFRB and/or IGF2R expression.

In embodiments of the present invention the binding molecule according to the present invention comprises at least two single variable domains, that are independently from one another, able to specifically bind a PDGFRB or IGF2R receptor of an activated myo-fibroblast and which domains are linked via a linker comprising a cis-platinum (II) complex or a maleimide modified linker moiety to a therapeutic molecule chosen from a kinase inhibitor, such as a Rho-kinase inhibitor, Jak-2 kinase inhibitor, neprilysin inhibitor or angiotensin II Receptor antagonist, or a cytotoxic molecule, preferably such a kinase inhibitor. Preferably, the cis-platinum (II) complex of the linker comprises an inert bidentate moiety, most preferably ethane-1,2-diamine. Furthermore, in embodiments, single variable domains that specifically bind to an IGF2R receptor comprise a CDR 1 sequence according to SEQ ID NO: 275, a CDR 2 sequence according to SEQ ID NO: 277 and a CDR 3 sequence according to SEQ ID NO: 279 or comprise a CDR 1 sequence according to SEQ ID NO:251, a CDR 2 sequence according to SEQ ID NO: 253 and a CDR3 sequence according to SEQ ID NO: 255 (i.e. according to 13F11 and 13E8, respectively); in embodiments, single variable domains that specifically bind a PDGFRB receptor comprise a CDR 1 sequence according to SEQ ID NO: 11, a CDR 2 sequence according to SEQ ID NO: 13 and a CDR 3 sequence according to SEQ ID NO: 15 or a CDR 1 sequence according to SEQ ID NO: 155, a CDR 2 sequence according to SEQ ID NO: 157 and a CDR 3 sequence according to SEQ ID NO: 159 (i.e. according to SP02P and SP26P, respectively). Furthermore, said binding molecule preferably also comprises a so-called half-life extender, which is preferably chosen from an albumin binding domain, albumin binding VHH or an antibody Fc tail or fragment thereof, more preferably, is an albumin binding domain.

In the Examples below, amongst others, specific species of these (preferred) embodiments have been described in more detail.

### NUMBERED EMBODIMENTS I.-XLVIII ACCORDING TO THE INVENTION

i. A binding molecule comprising at least two single variable antibody domains and at least one diagnostic molecule or therapeutic molecule, wherein the at least two single variable antibody domains are able to specifically bind to a transmembrane receptor expressed on a fibrogenic effector cell, which fibrogenic effector cell is characterized by the expression of alpha smooth muscle actin and/or stress fibers discernable by immune fluorescence microscopy, such as any one or more of an activated: portal fibroblast, hepatic stellate cell, pericyte, alveolar smooth muscle cell, alveolar fibroblast, interstitial fibroblast, mesangial cell, dermal fibroblast, mucosal smooth muscle cell, intestinal (myo-) fibroblast, interstitial cells of Cajal, or any precursor of a tumor stromal fibroblast in cancer, preferably a fibrogenic effector cell selected from any one or more of an activated: alveolar smooth muscle cell in the lung, alveolar fibroblast in the lung and/or pericyte in the lung, interstitial fibroblast in the kidney, pericyte in the kidney and/or mesangial cell in the kidney, dermal fibroblast in the skin and/or pericyte in the skin, mucosal smooth muscle cell in the intestine, intestinal (myo-) fibroblast in the intestine, interstitial cell of Cajal in the intestine, or pericyte in the intestine, or any precursor of a tumor stromal fibroblast in cancer, wherein the at least two single variable antibody domains are variable domains of a heavy chain only antibody (VHH), wherein the transmembrane receptor is an insulin-like growth factor 2 receptor (IGF2R) or wherein the transmembrane receptor is a platelet-derived growth factor beta receptor (PDGFRB).
ii. The binding molecule according to embodiment i, wherein the transmembrane receptor is an insulin-like growth factor 2 receptor (IGF2R).
iii. The binding molecule according to embodiment i or ii, wherein the binding molecule comprises a first VHH able to specifically bind to IGF2R and comprises a second VHH able to specifically bind to IGF2R.
iv. The binding molecule according to embodiment ii or iii, wherein the binding molecule comprises at least two single variable antibody domains, preferably two single variable antibody domains, that are, independently from one another, able to specifically bind to the transmembrane receptor IGF2R, expressed on the fibrogenic effector cell.
v. The binding molecule according to embodiment iii or iv, wherein the binding molecule is a multiparatopic binding molecule, preferably a biparatopic binding molecule, preferably wherein binding of the first VHH comprised by the binding molecule to IGF2R enhances binding of the second VHH comprised by the binding molecule to IGF2R and preferably vice versa, and/or preferably wherein binding of the first VHH and the second VHH to IGF2R enhances internalization of the binding molecule compared to internalization of a second binding molecule comprising either the first VHH or the second VHH only.
vi. The binding molecule according to any one of the embodiments iii-v, wherein the binding molecule is a multiparatopic binding molecule, preferably a biparatopic binding molecule, wherein binding of the first VHH to IGF2R expressed on a cell of a first species and to IGF2R expressed on a cell of a second species enhances binding of the second VHH to IGF2R expressed on said cell of the first species and to IGF2R expressed on said cell of the second species, and preferably vice versa,
vii. The binding molecule according to any one of the embodiments iii-vi, wherein the binding molecule is a biparatopic binding molecule, wherein at least one, preferably both, of the first VHH and the second VHH is able to compete for binding to IGF2R with any one or more of single domain antibodies having the sequence of 13A8 with SEQ ID NO: 257, 13A12 with SEQ ID NO: 289, 13C11 with SEQ ID NO: 297, 13E8 with SEQ ID NO: 249, 13F11 with SEQ ID NO: 273 or 13G10 with SEQ ID NO: 305, wherein preferably the first VHH and the second VHH do not both compete with the same single domain antibody having the sequence of 13A8, 13A12, 13C11, 13E8, 13F11 or 13G10.
viii. The binding molecule according to any one of the embodiments iii-vii, comprising the first VHH capable of specifically binding to IGF2R, the first VHH comprising a CDR 1 sequence according SEQ ID NO: 251, a CDR 2 sequence according to SEQ ID NO: 253 and a CDR 3 sequence according to SEQ ID NO: 255, and comprising the second VHH capable of specifically binding to IGF2R, the second VHH comprising any one of: a CDR 1 sequence according SEQ ID NO: 275, a CDR 2 sequence according to SEQ ID NO: 277 and a CDR 3 sequence according to SEQ ID NO: 279; or a CDR 1 sequence according SEQ ID NO: 259, a CDR 2 sequence according to SEQ ID NO: 261 and a CDR 3 sequence according to SEQ ID NO: 263; or a CDR 1 sequence according SEQ ID NO: 291, a CDR 2 sequence according to SEQ ID NO: 293 and a CDR 3 sequence according to SEQ ID NO: 295; or a CDR 1 sequence according SEQ ID NO: 299, a CDR 2 sequence according to SEQ ID NO: 301 and a CDR 3 sequence according to SEQ ID NO: 303; or a CDR 1 sequence according SEQ ID NO: 307, a CDR 2 sequence according to SEQ ID NO: 309 and a CDR 3 sequence according to SEQ ID NO: 311, or any of the combinations of CDR1, CDR2 and CDR3 sequences wherein, independently, at most 4 amino acids, preferably at most 3, more preferably at most 2, most preferably at most 1, have/has been conservatively substituted, or any of the combinations of CDR1, CDR2 and CDR3 sequences wherein, independently, 85-99% of amino acids, preferably 90-99%, more preferably 95-99%, most preferably 97-99%, have been conservatively substituted.
ix. The binding molecule according to any one of the embodiments iii-vii, comprising the first VHH capable of specifically binding to IGF2R, the first VHH comprising a CDR 1 sequence according SEQ ID NO: 251, a CDR 2 sequence according to SEQ ID NO: 253 and a CDR 3 sequence according to SEQ ID NO: 255, and comprising the second VHH capable of specifically binding to IGF2R, the second VHH comprising a CDR 1 sequence according SEQ ID NO: 275, a CDR 2 sequence according to SEQ ID NO: 277 and a CDR 3 sequence according to SEQ ID NO: 279, or wherein, independently, at most 4 amino acids in one or two of the CDR 1 sequences, one or two of the CDR 2 sequences and/or one or two of the CDR 3 sequences, preferably at most 3, more preferably at most 2, most preferably at most 1, have/has been conservatively substituted, or wherein, independently, 85-99% of amino acids in one or two of the CDR 1 sequences, one or two of the CDR 2 sequences and/or one or two of the CDR 3 sequences, preferably 90-99%, more preferably 95-99%, most preferably 97-99%, have been conservatively substituted.
x. The binding molecule according to any one of the embodiments iii-viii, comprising the first VHH capable of specifically binding to IGF2R, the first VHH having the sequence of 13E8 with SEQ ID NO: 249, and comprising the second VHH capable of specifically binding to IGF2R, the second VHH having the sequence of any one of: 13A8 with SEQ ID NO: 257, 13A12 with SEQ ID NO: 289, 13C11 with SEQ ID NO: 297, 13F11 with SEQ ID NO: 273 and 13G10 with SEQ ID NO: 305, preferably comprising the first VHH having the sequence of 13E8 with SEQ ID NO: 249 and the second VHH having the sequence of 13F11 with SEQ ID NO: 273.
xi. The binding molecule according to embodiment i, wherein the transmembrane receptor is a platelet-derived growth factor beta receptor (PDGFRB).
xii. The binding molecule according to embodiment i or xi, wherein the binding molecule comprises a first VHH able to specifically bind to PDGFRB and comprises a second VHH able to specifically bind to PDGFRB.
xiii. The binding molecule according to embodiment xi or xii, wherein the binding molecule comprises at least two single variable antibody domains, preferably two single variable antibody domains, that are, independently from one another, able to specifically bind to the transmembrane receptor PDGFRB, expressed on the fibrogenic effector cell.
xiv. The binding molecule according to embodiment xii or xiii, wherein the binding molecule is a multiparatopic binding molecule, preferably a biparatopic binding molecule, preferably wherein binding of the first VHH comprised by the binding molecule to PDGFRB enhances binding of the second VHH comprised by the binding molecule to PDGFRB and preferably vice versa, and/or preferably wherein binding of the first VHH and the second VHH to PDGFRB enhances internalization of the binding molecule compared to internalization of a second binding molecule comprising either the first VHH or the second VHH only.
xv. The binding molecule according to any one of the embodiments xii-xiv, wherein the binding molecule is a multiparatopic binding molecule, preferably a biparatopic binding molecule, wherein binding of the first VHH to PDGFRB expressed on a cell of a first species and to PDGFRB expressed on a cell of a second species enhances binding of the second VHH to PDGFRB expressed on said cell of the first species and to PDGFRB expressed on said cell of the second species, and preferably vice versa,
xvi. The binding molecule according to any one of the embodiments xii-xv, wherein the binding molecule is a biparatopic binding molecule, wherein at least one, preferably both, of the first VHH and the second VHH is able to compete for binding to PDGFRB with any one or more of single domain antibodies having the sequence of SP01P with SEQ ID NO: 1, SP02P with SEQ ID NO: 9, SP05P with SEQ ID NO: 25, SP07P with SEQ ID NO: 33, SP08P with SEQ ID NO: 41, SP10P with SEQ ID NO: 49, SP12P with SEQ ID NO: 65, SP13P with SEQ ID NO: 73, SP14P with SEQ ID NO: 81, SP20P with SEQ ID NO: 113, SP26P with SEQ ID NO: 153 or SP28P with SEQ ID NO: 169, wherein preferably the first VHH and the second VHH do not both compete with the same single domain antibody having the sequence of SP01P, SP02P, SP05P, SP07P, SP08P, SP10P, SP12P, SP13P, SP14P, SP20P, SP26P or SP28P.
xvii. The binding molecule according to any one of the embodiments xii-xvi, comprising the first VHH capable of specifically binding to PDGFRB, the first VHH comprising a CDR 1 sequence according SEQ ID NO: 155, a CDR 2 sequence according to SEQ ID NO: 157 and a CDR 3 sequence according to SEQ ID NO: 159, and comprising the second VHH capable of specifically binding to PDGFRB, the second VHH comprising any one of: a CDR 1 sequence according SEQ ID NO: 3, a CDR 2 sequence according to SEQ ID NO: 5 and a CDR 3 sequence according to SEQ ID NO: 7; or a CDR 1 sequence according SEQ ID NO: 11, a CDR 2 sequence according to SEQ ID NO: 13 and a CDR 3 sequence according to SEQ ID NO: 15; or a CDR 1 sequence according SEQ ID NO: 35, a CDR 2 sequence according to SEQ ID NO: 37 and a CDR 3 sequence according to SEQ ID NO: 39; or a CDR 1 sequence according SEQ ID NO: 75, a CDR 2 sequence according to SEQ ID NO: 77 and a CDR 3 sequence according to SEQ ID NO: 79; or any of the combinations of CDR1, CDR2 and CDR3 sequences wherein, independently, at most 4 amino acids, preferably at most 3, more preferably at most 2, most preferably at most 1, have/has been conservatively substituted, or any of the combinations of CDR1, CDR2 and CDR3 sequences wherein, independently, 85-99% of amino acids, preferably 90-99%, more preferably 95-99%, most preferably 97-99%, have been conservatively substituted.
xviii. The binding molecule according to any one of the embodiments xii-xvi, comprising the first VHH capable of specifically binding to PDGFRB, the first VHH comprising a CDR 1 sequence according SEQ ID NO: 155, a CDR 2 sequence according to SEQ ID NO: 157 and a CDR 3 sequence according to SEQ ID NO: 159, and comprising the second VHH capable of specifically binding to PDGFRB, the second VHH comprising a CDR 1 sequence according SEQ ID NO: 11, a CDR 2 sequence according to SEQ ID NO: 13 and a CDR 3 sequence according to SEQ ID NO: 15, or wherein, independently, at most 4 amino acids in one or two of the CDR 1 sequences, one or two of the CDR 2 sequences and/or one or two of the CDR 3 sequences, preferably at most 3, more preferably at most 2, most preferably at most 1, have/has been conservatively substituted, or wherein, independently, 85-99% of amino acids in one or two of the CDR 1 sequences, one or two of the CDR 2 sequences and/or one or two of the CDR 3 sequences, preferably 90-99%, more preferably 95-99%, most preferably 97-99%, have been conservatively substituted.
xix. The binding molecule according to any one of the embodiments xii-xvii, comprising the first VHH capable of specifically binding to PDGFRB, the first VHH having the sequence of SP26P with SEQ ID NO: 153, and comprising the second VHH capable of specifically binding to PDGFRB, the second VHH having the sequence of any one of: SP01P with SEQ ID NO: 1, SP02P with SEQ ID NO: 9, SP07P with SEQ ID NO: 33 and SP13P with SEQ ID NO: 73, preferably comprising the first VHH having the sequence of SP26P with SEQ ID NO: 153 and the second VHH having the sequence of SP02P with SEQ ID NO: 9.
xx. A binding molecule according to any one of the embodiments i-xix comprising at least two single variable antibody domains, wherein the at least two single variable antibody domains are separated by a linker amino acid sequence.
xxi. A binding molecule according to any one of the embodiments i-xx, wherein the binding molecule comprises an N-terminal or a C-terminal cysteine or histidine residue, preferably an N-terminal or a C-terminal cysteine residue.
xxii. A binding molecule according to any one of the embodiments i-xxi, wherein the binding molecule comprises a half-life extender.
xxiii. A binding molecule according to embodiment xxii, wherein the half-life extender comprises or consists of one or more groups, residues, moieties or binding units that provide the binding molecule with increased half-life compared to a binding molecule without a half-life extender.
xxiv. A binding molecule according to embodiment xxiii, wherein the groups, residues, moieties or binding units that provide the binding molecule with increased half-life are an albumin binding domain or an albumin binding VHH or a fragment thereof.
XXV. A binding molecule according to any one of the embodiments i-xxiv, wherein binding of the binding molecule to said receptor enables induction of ligand internalization or antibody internalization.
xxvi. A binding molecule according to embodiment xxv, wherein binding of one of the single variable antibody domains to its antigen modulates the binding of the other single variable antibody domain to its antigen.
xxvii. A binding molecule according to any one of the embodiments i-xxvi, wherein the therapeutic molecule is able to inhibit any one or more of contractile activity, fibrogenic activity, chemotactic activity and pro-inflammatory activity of the effector cell.
xxviii. A binding molecule according to any one of the embodiments i-xxvii, wherein the therapeutic molecule is a kinase inhibitor, preferably selected from Rho-kinase inhibitors, JAK-2 inhibitors or neprilysin inhibitors, or Angiotensin II Receptor antagonists, such as losartan, preferably wherein the therapeutic molecule is a Rho-kinase inhibitor or a JAK-2 inhibitor.
xxix. Nucleic acid that encodes amino acid residues of at least part of a binding molecule, and preferably the amino acid residues of the binding molecule, according to any of the embodiments i-xxviii.
xxx. A host cell for expression of amino acid residues of at least part of a binding molecule, and preferably the amino acid residues of the binding molecule, according to any one of the embodiments i-xxviii. comprising a nucleic acid according to embodiment xxix.
xxxi. A method for producing a binding molecule according to any one of the embodiments i-xxviii, comprising culturing a host cell according to embodiment xxx, allowing for expression of amino acid residues of at least part of said binding molecule, preferably the amino acid residues of the binding molecule, harvesting the expressed amino acid residues of the at least part of the binding molecule or of the binding molecule, and coupling the therapeutic molecule or the diagnostic molecule to said amino acid residues of part of said binding molecule or of the binding molecule, optionally through a linker.
xxxii. Binding molecule according to any one of the embodiments i-xxviii for use as a medicament.
xxxiii. Binding molecule according to any one of the embodiments i-xxviii, for use in a method for the prevention and/or treatment of a disease or disorder associated with or characterized by the upregulation of PDGFRB and/or IGF2R expression.
xxxiv. Binding molecule for use according to embodiment xxxiii, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, preferably any one or more of: lung fibrosis, renal fibrosis, systemic sclerosis/scleroderma, inflammatory bowel disease and a cancer relating to a (sclerosing) malignant tumor such as breast cancer, lung cancer, colon cancer and prostate cancer.
xxxv. Binding molecule for use according to embodiment xxxiii or xxxiv, wherein the prevention and/or the treatment is the prevention and/or the treatment of a (secondary) clinical manifestation that is attributable to liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.
xxxvi. Binding molecule for use according to embodiment xxxv, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.
xxxvii. Pharmaceutical composition comprising at least one binding molecule according to any one of the embodiments i-xxviii and at least one pharmaceutically acceptable excipient.
xxxviii. Pharmaceutical composition of embodiment xxxvii, for use as a medicament.
xxxix. Pharmaceutical composition of embodiment xxxvii, for use in a method for the prevention and/or treatment of a disease or disorder associated with or characterized by the upregulation of PDGFRB and/or IGF2R expression.
xl. Pharmaceutical composition for use according to embodiment xxxix, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, preferable any one or more of: lung fibrosis, renal fibrosis, systemic sclerosis/scleroderma, inflammatory bowel disease and a cancer relating to a (sclerosing) malignant tumor such as breast cancer, lung cancer, colon cancer and prostate cancer.
xli. Pharmaceutical composition for use according to embodiment xxxix or xl, wherein the prevention and/or the treatment is the prevention and/or the treatment of a (secondary) clinical manifestation that is attributable to said liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.
xlii. Pharmaceutical composition for use of embodiment xl1, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.
xliii. Pharmaceutical composition comprising at least one binding molecule according to any one of the embodiments i-xxviii and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor.
xliv. A pharmaceutical composition according to embodiment xliii, for use as an adjuvant treatment of secondary hemodynamic manifestations of fibrotic disease, such as portal hypertension and pulmonary hypertension.
xlv. A pharmaceutical composition according to embodiment xliv, for use in the treatment of an acute manifestation of: portal hypertension such as acute esophageal bleeding or pulmonary hypertension such as acute cardiac decompensation.
xlvi. A binding molecule according to any of the embodiments i-xxviii, comprising a diagnostic molecule, wherein the diagnostic molecule is an imaging agent.
xlvii. Diagnostic composition comprising at least one binding molecule according to embodiment xlvi and a diluent and/or excipient.
xlviii. Use of the binding molecule of embodiment xlvi or the diagnostic composition of embodiment xlvii in a method for diagnosing of a disease or disorder associated with or characterized by the upregulation of PDGFRB and/or IGF2R expression.

Particularly preferred binding molecules of the invention are biparatopic binding molecules comprising either a first and a second VHH domain binding to IGF2R, or comprising a first and a second VHH domain binding to PDGFRB. Preferably, such binding molecule comprises a half-life extender, preferably an albumin binding domain (ABD), for example an albumin binding VHH. For a preferred binding molecule of the invention, the therapeutic molecule is a kinase inhibitor, preferably selected from Rho-kinase inhibitors, JAK-2 inhibitors or neprilysin inhibitors, or Angiotensin II Receptor antagonists, such as losartan. More preferably, for the binding molecule of the invention the therapeutic molecule is a Rho-kinase inhibitor or a JAK-2 inhibitor. According to the invention, typically suitable therapeutic molecules for conjugating to the VHH domains of the binding molecule are Y27632 which is a ROCK (Rho-kinase) inhibitor, and pacritinib which is a JAK-2 inhibitor. For the binding molecule of the invention comprising a therapeutic molecule, the ratio between the therapeutic molecule and the two VHH domains can be 1 to 1 or for example any ratio selected from 2:1 and 64:1, such as 3:1, 4:1, 5:1, 6:1, 7:1, 8:1, 10:1, 12:1, 16:1, 20:1, 24:1, 30:1, 32:1, 40:1, 50:1 and any ratio in between. That is to say, a binding molecule comprises a single copy of the therapeutic molecule, or a binding molecule comprises multiple copies of the therapeutic molecule, such as two, three, four, six, eight, ten, twelve, sixteen copies.

Preferred biparatopic binding molecules of the invention, comprising two VHH domains, are 13E8-13F11, 13F11-13E8, 13E8-13F11-ABD, 13F11-13E8-ABD, 13E8-13F11 (Y27632), 13E8-13F11-ABD (Y27632), 13E8-13F11 (pacritinib), 13E8-13F11-ABD (pacritinib), 13F11-13E8 (Y27632), 13F11-13E8-ABD (Y27632), 13F11-13E8 (pacritinib), 13F11-13E8-ABD (pacritinib), comprising optionally (and preferred) an albumin binding domain (ABD) and optionally (and preferred) a therapeutic molecule such as Y27632 or pacritinib as indicated. Preferred are 13E8-13F11-ABD (pacritinib), 13E8-13F11-ABD (Y27632), 13F11-13E8-ABD (Y27632) and 13F11-13E8-ABD (pacritinib), more preferred are 13F11-13E8 (Y27632), 13F11-13E8-ABD (Y27632), 13F11-13E8 (pacritinib) and 13F11-13E8-ABD (pacritinib). Particularly preferred is the binding molecule 13F11-13E8 (Y27632) or 13F11-13E8-ABD (Y27632).

Preferred biparatopic binding molecules of the invention, comprising two VHH domains, are SP02P-SP26P, SP26P-SP02P, SP02P-SP26P-ABD, SP26P-SP02P-ABD, SP02P-SP26P(Y27632), SP02P-SP26P-ABD (Y27632), SP02P-SP26P (pacritinib), SP02P-SP26P-ABD (pacritinib), SP26P-SP02P(Y27632), SP26P-SP02P-ABD (Y27632), SP26P-SP02P (pacritinib), SP26P-SP02P-ABD (pacritinib), comprising optionally (and preferred) an albumin binding domain (ABD) and optionally (and preferred) a therapeutic molecule such as Y27632 or pacritinib as indicated. Preferred are SP02P-SP26P-ABD (Y27632), SP02P-SP26P-ABD (pacritinib), SP26P-SP02P-ABD (Y27632) and SP26P-SP02P-ABD (pacritinib), more preferred are SP02P-SP26P(Y27632), SP02P-SP26P-ABD (Y27632), SP02P-SP26P (pacritinib) and SP02P-SP26P-ABD (pacritinib).

Surprisingly, the inventors provided relatively high-affinity biparatopic binding molecules comprising a first and a second VHH domain, wherein the high affinity was apparent despite of the respective lower binding affinities of the two corresponding monomeric VHHs to respective substrates (target receptor), demonstrating a synergistic contribution of each of the two monomeric VHHs to the binding characteristics of the resulting biparatopic constructs, and hence superiority of the biparatopic design over monomeric VHHs. In particular the synergy is apparent for binding molecules targeting the IGF2R, such as binding molecules comprising VHH domains 13E8 and 13F11.

### NUMBERED EMBODIMENTS 1-59 ACCORDING TO THE INVENTION

1. A binding molecule comprising at least one single variable antibody domain and at least one diagnostic molecule or therapeutic molecule, wherein the at least one single variable antibody domain is able to specifically bind to a transmembrane receptor expressed on a fibrogenic effector cell, generally characterized by the expression of alpha smooth muscle actin and/or stress fibers discernable by (immune fluorescence) microscopy or similar methods, such as any one or more of an activated: portal fibroblast, hepatic stellate cell, pericyte, alveolar smooth muscle cell, alveolar fibroblast, interstitial fibroblast, mesangial cell, dermal fibroblast, mucosal smooth muscle cell, intestinal (myo-) fibroblast, interstitial cells of Cajal, or any precursor of tumor stromal fibroblasts in cancer, preferably a fibrogenic effector cell selected from any one or more of an activated: portal fibroblast, hepatic stellate cell or pericyte in the liver, alveolar smooth muscle cell, alveolar fibroblast or pericyte in the lung, interstitial fibroblast, pericyte or mesangial cell in the kidney, dermal fibroblast or pericyte in the skin, mucosal smooth muscle cell, intestinal (myo-) fibroblast, interstitial cell of Cajal, or pericyte in the intestine, or any precursor of a tumor stromal fibroblast in cancer.
2. A binding molecule according to embodiment 1, wherein the binding molecule comprises at least two single variable antibody domains that are, independently from one another, able to specifically bind to the transmembrane receptor expressed on the fibrogenic effector cell.
3. A binding molecule according to embodiment 1 or 2, wherein the binding molecule is a multiparatopic binding molecule, preferably a biparatopic binding molecule.
4. A binding molecule according to any one of the embodiments 1-3, wherein the binding molecule is a multivalent binding molecule, preferably a bivalent binding molecule.
5. A binding molecule according to any one of the previous numbered embodiments 1-4, wherein the binding molecule is a multispecific binding molecule, preferably a bispecific binding molecule.
6. A binding molecule according to any one of the previous numbered embodiments 1-5, wherein the transmembrane receptor is a platelet-derived growth factor beta receptor (PDGFRB) or an insulin-like growth factor 2 receptor (IGF2R).
7. A binding molecule according to any one of the previous numbered embodiments 1-6, wherein the therapeutic molecule or diagnostic molecule is bound to the at least one single variable antibody domain by a linker and/or spacer.
8. A binding molecule according to any one of the previous numbered embodiments 1-7, wherein the linker and/or the spacer comprises or consists of a transition metal complex.
9. A binding molecule according to embodiment 8, wherein the linker and/or the spacer comprises a cis-platinum (II) complex.
10. A binding molecule according embodiment 8 or 9, wherein the linker and/or the spacer comprises a cis-platinum (II) complex comprising an inert bidentate moiety, preferably ethane-1,2-diamine.
11. A binding molecule according to any one of the previous numbered embodiments 1-10s, wherein the binding molecule comprises a half-life extender.
12. A binding molecule according to embodiment 11, wherein the half-life extender comprises or consists of one or more groups, residues, moieties or binding units that provide the binding molecule with increased half-life compared to a binding molecule without a half-life extender.
13. A binding molecule according to embodiment 12, wherein the groups, residues, moieties or binding units that provide the binding molecule with increased half-life are an albumin binding domain, an albumin binding VHH or an antibody Fc tail or a fragment thereof.
14. A binding molecule according to any one of the previous numbered embodiments 1-13, wherein binding of the binding molecule to said receptor enables induction of ligand internalization or antibody internalization.
15. A binding molecule according to any one of the previous numbered embodiments 1-14, wherein the at least one single variable antibody domain is, and/or the at least two single variable antibody domains are, independently from one another, selected from the group consisting of an immunoglobulin single variable domain antibody (ISVD), a variable domain of a heavy chain (VH), a variable domain of a heavy chain only antibody (VHH), a domain antibody (dAb), and a single domain antibody (sdAb).
16. A binding molecule according to any one of the previous numbered embodiments 1-15, wherein the at least two single variable antibody domains are both single variable domain antibodies, preferably variable domains of heavy chain only antibodies.
17. A binding molecule according to embodiment 16, wherein binding of one of the single variable antibody domains to its antigen modulates the binding of the other single variable antibody domain to its antigen.
18. A binding molecule according to any one of the previous numbered embodiments 1-17, comprising at least two single variable antibody domains capable of specifically binding to PDGFRB.
19. A binding molecule according to any one of the previous numbered embodiments 1-18, comprising at least two single variable antibody domains capable of specifically binding to IGF2R.
20. A binding molecule according to any one of the previous numbered embodiments 1-19, comprising at least one single variable antibody domain capable of specifically binding to PDGFRB and at least one single variable antibody domain capable of specifically binding to IGF2R.
21. A binding molecule according to any one of the previous numbered embodiments 1-20 comprising at least a first single variable antibody domain that is able to compete with a single domain antibody having any of the sequence of SP14P (SEQ ID NO: 81) or SP02P (SEQ ID NO: 9) or SP05P (SEQ ID NO: 25) or SP12P (SEQ ID NO: 65) or SP26P (SEQ ID NO: 153) or SP28P (SEQ ID NO: 169) in specific binding to the PDGFRB.
22. A binding molecule according to any one of the previous numbered embodiments 1-21, comprising at least one single variable antibody domain that is able to compete with a single domain antibody having the sequence of SP02P (SEQ ID NO: 9) or SP26P (SEQ ID NO: 153) in specific binding to the PDGFRB.
23. A binding molecule according to any one of the previous numbered embodiments 1-22 comprising at least a second single variable antibody domain that is able to compete with a single domain antibody having any of the sequence of SP14P or SP02P or SP05P or SP12P, wherein preferably the first and the second single variable antibody domains do not both compete with the same single domain antibody having the sequence of SP14P or SP02P or SP05P or SP12P.
24. A binding molecule according to any one of the previous numbered embodiments 1-23, comprising at least one single variable antibody domain that is able to compete with a single domain antibody having the sequence of 13E8 (SEQ ID NO: 249) or 13F11 (SEQ ID NO: 273) in specific binding to the IGF2R.
25. A binding molecule according to the previous numbered embodiments 1-24, comprising at least a second single variable antibody domain that is able to compete with a single domain antibody having the sequence of 13E8 or 13F11, wherein preferably the first and the second single variable antibody domains do not both compete with the same single domain antibody having the sequence of 13E8 or 13F11.
26. A binding molecule according to any one of the previous numbered embodiments 1-25, comprising at least one single variable antibody domain capable of specifically binding to PDGFRB, the at least one single variable antibody domain comprising a CDR 1 sequence according SEQ ID NO: 83, a CDR 2 sequence according to SEQ ID NO: 85 and a CDR 3 sequence according to SEQ ID NO: 87; or a CDR 1 sequence according SEQ ID NO: 3, a CDR 2 sequence according to SEQ ID NO: 5 and a CDR 3 sequence according to SEQ ID NO: 7; or a CDR 1 sequence according SEQ ID NO: 11, a CDR 2 sequence according to SEQ ID NO: 13 and a CDR 3 sequence according to SEQ ID NO: 15; or a CDR 1 sequence according SEQ ID NO: 35, a CDR 2 sequence according to SEQ ID NO: 37 and a CDR 3 sequence according to SEQ ID NO: 39; or a CDR 1 sequence according SEQ ID NO: 75, a CDR 2 sequence according to SEQ ID NO: 77 and a CDR 3 sequence according to SEQ ID NO: 79; or a CDR 1 sequence according SEQ ID NO: 27, a CDR 2 sequence according to SEQ ID NO: 29 and a CDR 3 sequence according to SEQ ID NO: 31; or a CDR 1 sequence according SEQ ID NO: 43, a CDR 2 sequence according to SEQ ID NO: 45 and a CDR 3 sequence according to SEQ ID NO: 47; or a CDR 1 sequence according SEQ ID NO: 51, a CDR 2 sequence according to SEQ ID NO: 53 and a CDR 3 sequence according to SEQ ID NO: 55; or a CDR 1 sequence according SEQ ID NO: 115, a CDR 2 sequence according to SEQ ID NO: 117 and a CDR 3 sequence according to SEQ ID NO: 119; or a CDR 1 sequence according SEQ ID NO: 67, a CDR 2 sequence according to SEQ ID NO: 69 and a CDR 3 sequence according to SEQ ID NO: 71; or a CDR 1 sequence according SEQ ID NO: 155, a CDR 2 sequence according to SEQ ID NO: 157 and a CDR 3 sequence according to SEQ ID NO: 159; or any of the combinations of CDR1, CDR2 and CDR3 sequences wherein, independently, at most 4 amino acids, more preferably at most 3, more preferably at most 2, most preferably at most 1, has been conservatively substituted.
27. A binding molecule according to any one of the previous numbered embodiments 1-27, comprising at least one single variable antibody domain capable of specifically binding to IGF2R, the at least one single variable antibody domain comprising a CDR 1 sequence according SEQ ID NO: 251, a CDR 2 sequence according to SEQ ID NO: 253 and a CDR 3 sequence according to SEQ ID NO: 255; or a CDR 1 sequence according SEQ ID NO: 275, a CDR 2 sequence according to SEQ ID NO: 277 and a CDR 3 sequence according to SEQ ID NO: 279; or a CDR 1 sequence according SEQ ID NO: 259, a CDR 2 sequence according to SEQ ID NO: 261 and a CDR 3 sequence according to SEQ ID NO: 263; or a CDR 1 sequence according SEQ ID NO: 291, a CDR 2 sequence according to SEQ ID NO: 293 and a CDR 3 sequence according to SEQ ID NO: 295; or a CDR 1 sequence according SEQ ID NO: 299, a CDR 2 sequence according to SEQ ID NO: 301 and a CDR 3 sequence according to SEQ ID NO: 303; or a CDR 1 sequence according SEQ ID NO: 307, a CDR 2 sequence according to SEQ ID NO: 309 and a CDR 3 sequence according to SEQ ID NO: 311, or any of the combinations of CDR1, CDR2 and CDR3 sequences wherein, independently, at most 4 amino acids, more preferably at most 3, more preferably at most 2, most preferably at most 1, has been conservatively substituted.
28. A binding molecule according to any one of the previous numbered embodiments 1-27, comprising at least one single variable antibody domain capable of specifically binding to PDGFRB, wherein the at least one single variable antibody domain comprises or consists of any one of SEQ ID NOs: 81, 1, 9, 33, 73, 25, 41, 49, 65, or 113.
29. A binding molecule according to any one of the previous numbered embodiments 1-28, comprising at least one single variable antibody domain capable of specifically binding to IGF2R, wherein the at least one single variable antibody domain comprises or consists of any one of SEQ ID NOs: 249, 273, 257, 289, 297, or 305.
30. A binding molecule according to the previous embodiment comprising at least two single variable antibody domains, wherein at least one single variable antibody domain comprises or consists of SEQ ID NOs: 81 and at least one single variable antibody domain comprises or consists of any one of SEQ ID NOs: 1, 9, 33, 73, 25, 41, 49, 65, or 113; or wherein at least one single variable antibody domain comprises or consists of any one of SEQ ID NOs: 1, 9, 33, or 73 and at least one single variable antibody domain comprises or consists of any one of SEQ ID NOs: 25, 41, 49, 65, or 113; or wherein at least one single variable antibody domain comprises or consists of any one of SEQ ID NOs: 25, 41, 49 and at least one single variable antibody domain comprises or consists of any one of SEQ ID NOs: 65 or 113.
31. A binding molecule according to embodiment 29 comprising at least two single variable antibody domains, wherein at least one single variable antibody domain comprises or consists of SEQ ID NOs: 249 and at least one single variable antibody domain comprises or consists of any one of SEQ ID NOs: 273, 257, 289, 297, or 305.
32. A binding molecule according to any one of the previous numbered embodiments 1-31, comprising at least two single variable antibody domains, wherein at least one single variable antibody domain comprises or consists of any one of SEQ ID NOs: 81, 1, 9, 33, 73, 25, 41, 49, 65, or 113 and at least one single variable antibody domain comprises or consists of any one of SEQ ID NOs: 249, 273, 257, 289, 297, or 305.
33. A binding molecule according to any one of the previous numbered embodiments 1-32 comprising at least two single variable antibody domains, wherein the at least two single variable antibody domains are separated by a linker amino acid sequence.
34. A binding molecule according to any one of the previous numbered embodiments 1-33, wherein the at least one single variable antibody domain is, or the at least two single variable antibody domains are, independently from one another, capable of specifically binding to their respective target with a dissociation constant (K_{D}) of 10E-5 to 10E-12 moles/liter or less, and preferably of 10E-7 to 10E-12 moles/liter or less and more preferably of 10E-9 to 10E-12 moles/liter.
35. A binding molecule according to any one of the previous numbered embodiments 1-34, wherein the therapeutic molecule is able to inhibit any one or more of contractile activity, fibrogenic activity, chemotactic activity and pro-inflammatory activity of the effector cell . . .
36. A binding molecule according to any one of the previous numbered embodiments 1-35, wherein the therapeutic molecule is a kinase inhibitor, preferably selected from Rho-kinase inhibitors, JAK-2 inhibitors or neprilysin inhibitors, or Angiotensin II Receptor antagonists, such as losartan.
37. A binding molecule according to any one of the previous numbered embodiments 1-36, wherein the therapeutic molecule is a cytotoxic molecule.
38. Binding molecule according to any one of the previous numbered embodiments 1-37, wherein binding of the binding molecule to the transmembrane receptor does not induce the receptor's intracellular signaling cascade.
39. Binding molecule according to any one of the previous numbered embodiments 1-38, wherein binding of the binding molecule to the transmembrane receptor leads to receptor mediated endocytosis and cytosolic release of the drug or cytotoxic molecule.
40. A binding molecule according to any one of the previous numbered embodiments 1-39, wherein the binding molecule comprises an N-terminal or a C-terminal cysteine or histidine residue, preferably an N-terminal or a C-terminal cysteine residue.
41. A dimeric binding molecule comprising at least two binding molecules according to the previous embodiment 40, wherein the at least two binding molecules have formed a dimer, preferably through the N-terminal or C-terminal cysteine residues.
42. Binding molecule according to any one of the previous numbered embodiments 1-41, for use as a medicament.
43. Binding molecule according to any one of the embodiments 1-41, for use in a method for the prevention and/or treatment of a disease or disorder associated with or characterized by the upregulation of PDGFRB and/or IGF2R expression.
44. Binding molecule for use according to embodiment 43, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, preferably any one or more of: lung fibrosis, renal fibrosis, systemic sclerosis/scleroderma, inflammatory bowel disease and a cancer relating to a (sclerosing) malignant tumor such as breast cancer, lung cancer, colon cancer and prostate cancer.
45. Binding molecule for use according to embodiment 44, wherein the prevention and/or the treatment is the prevention and/or the treatment of a (secondary) clinical manifestation that is attributable to said liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.
46. Binding molecule for use according to embodiment 45, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.
47. Nucleic acid that encodes at least part of a binding molecule according to any of the embodiments 1-41.
48. A host cell for expression of at least part of a binding molecule according to any one of the embodiments 1-41, comprising a nucleic acid according to embodiment 47.
49. A method for producing a binding molecule according to any one of the embodiments 1-41, comprising culturing a host cell according to embodiment 48, allowing for expression of at least part of said binding molecule, harvesting the binding molecule, and coupling the therapeutic molecule or the diagnostic molecule to said part of said binding molecule, optionally through a linker as defined in any one of the previous embodiments.
50. Pharmaceutical composition comprising at least one binding molecule according to any one of the embodiments 1-41 and at least one pharmaceutically acceptable excipient.
51. Pharmaceutical composition of embodiment 50, for use in a method for the prevention and/or treatment of a disease or disorder associated with or characterized by the upregulation of PDGFRB and/or IGF2R expression.
52. Pharmaceutical composition for use according to embodiment 51, wherein the disease or disorder is any one or more of: liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, preferable any one or more of: lung fibrosis, renal fibrosis, systemic sclerosis/scleroderma, inflammatory bowel disease and a cancer relating to a (sclerosing) malignant tumor such as breast cancer, lung cancer, colon cancer and prostate cancer.
53. Pharmaceutical composition for use according to embodiment 51 or 52, wherein the prevention and/or the treatment is the prevention and/or the treatment of a (secondary) clinical manifestation that is attributable to said liver fibrosis, cirrhosis, lung fibrosis, renal fibrosis, systemic sclerosis/scleroderma, inflammatory bowel disease and a cancer relating to a (sclerosing) malignant tumor such as breast cancer, lung cancer, colon cancer and prostate cancer, such as any one or more of the (secondary) clinical manifestations: portal hypertension in liver fibrosis and/or in cirrhosis and pulmonary hypertension such as pulmonary arterial hypertension and/or pulmonary venous hypertension in pulmonary fibrosis.
54. Pharmaceutical composition for use of embodiment 53, wherein the prevention and/or the treatment of a (secondary) clinical manifestation is the prevention and/or treatment of an acute complication of such (secondary) clinical manifestation, such as any one or more of: oesophageal bleeding in portal hypertension and/or cardiovascular failure, and/or pulmonary failure in pulmonary hypertension such as pulmonary arterial hypertension and/or pulmonary venous hypertension.
55. Pharmaceutical composition comprising at least one binding molecule according to any one of the embodiments 1-41 and at least one pharmaceutically acceptable excipient, further comprising at least one other compound useful in the treatment of a clinical manifestation of any one or more of liver fibrosis, lung fibrosis, renal fibrosis, systemic sclerosis/scleroderma, inflammatory bowel disease and an (sclerosing) malignant tumor.
56. A pharmaceutical composition according to embodiment 55, for use as an adjuvant treatment of secondary hemodynamic manifestations of fibrotic disease, such as portal hypertension and pulmonary hypertension.
57. A pharmaceutical composition according to embodiment 55, for use in the treatment of an acute manifestation of: portal hypertension such as acute esophageal bleeding or pulmonary hypertension such as acute cardiac decompensation.
58. A binding molecule according to any one of the embodiments 1-41, comprising a diagnostic molecule, wherein the diagnostic molecule is an imaging agent.
59. Diagnostic composition comprising at least one binding molecule according to the previous embodiment 58 and a diluent and/or excipient.

Particularly preferred binding molecules of the invention are biparatopic binding molecules comprising either a first and a second VHH domain binding to IGF2R, or comprising a first and a second VHH domain binding to PDGFRB. Preferably, such binding molecule comprises a half-life extender, preferably an albumin binding domain (ABD), for example an albumin binding VHH. For a preferred binding molecule of the invention, the therapeutic molecule is a kinase inhibitor, preferably selected from Rho-kinase inhibitors, JAK-2 inhibitors or neprilysin inhibitors, or Angiotensin II Receptor antagonists, such as losartan. More preferably, for the binding molecule of the invention the therapeutic molecule is a Rho-kinase inhibitor or a JAK-2 inhibitor. According to the invention, typically suitable therapeutic molecules for conjugating to the VHH domains of the binding molecule are Y27632 which is a ROCK (Rho-kinase) inhibitor, and pacritinib which is a JAK-2 inhibitor. For the binding molecule of the invention comprising a therapeutic molecule, the ratio between the therapeutic molecule and the two VHH domains can be 1 to 1 or for example any ratio selected from 2:1 and 64:1, such as 3:1, 4:1, 5:1, 6:1, 7:1, 8:1, 10:1, 12:1, 16:1, 20:1, 24:1, 30:1, 32:1, 40:1, 50:1 and any ratio in between. That is to say, a binding molecule comprises a single copy of the therapeutic molecule, or a binding molecule comprises multiple copies of the therapeutic molecule, such as two, three, four, six, eight, ten, twelve, sixteen copies.

Preferred biparatopic binding molecules of the invention, comprising two VHH domains, are 13E8-13F11, 13F11-13E8, 13E8-13F11-ABD, 13F11-13E8-ABD, 13E8-13F11 (Y27632), 13E8-13F11-ABD (Y27632), 13E8-13F11 (pacritinib), 13E8-13F11-ABD (pacritinib), 13F11-13E8 (Y27632), 13F11-13E8-ABD (Y27632), 13F11-13E8 (pacritinib), 13F11-13E8-ABD (pacritinib), comprising optionally (and preferred) an albumin binding domain (ABD) and optionally (and preferred) a therapeutic molecule such as Y27632 or pacritinib as indicated. Preferred are 13E8-13F11-ABD (pacritinib), 13E8-13F11-ABD (Y27632), 13F11-13E8-ABD (Y27632) and 13F11-13E8-ABD (pacritinib), more preferred are 13F11-13E8 (Y27632), 13F11-13E8-ABD (Y27632), 13F11-13E8 (pacritinib) and 13F11-13E8-ABD (pacritinib). Particularly preferred is the binding molecule 13F11-13E8 (Y27632) or 13F11-13E8-ABD (Y27632).

Preferred biparatopic binding molecules of the invention, comprising two VHH domains, are SP02P-SP26P, SP26P-SP02P, SP02P-SP26P-ABD, SP26P-SP02P-ABD, SP02P-SP26P(Y27632), SP02P-SP26P-ABD (Y27632), SP02P-SP26P (pacritinib), SP02P-SP26P-ABD (pacritinib), SP26P-SP02P(Y27632), SP26P-SP02P-ABD (Y27632), SP26P-SP02P (pacritinib), SP26P-SP02P-ABD (pacritinib), comprising optionally (and preferred) an albumin binding domain (ABD) and optionally (and preferred) a therapeutic molecule such as Y27632 or pacritinib as indicated. Preferred are SP02P-SP26P-ABD (Y27632), SP02P-SP26P-ABD (pacritinib), SP26P-SP02P-ABD (Y27632) and SP26P-SP02P-ABD (pacritinib), more preferred are SP02P-SP26P(Y27632), SP02P-SP26P-ABD (Y27632), SP02P-SP26P (pacritinib) and SP02P-SP26P-ABD (pacritinib).

Surprisingly, the inventors provided relatively high-affinity biparatopic binding molecules comprising a first and a second VHH domain, wherein the high affinity was apparent despite of the respective lower binding affinities of the two corresponding monomeric VHHs to respective substrates (target receptor), demonstrating a synergistic contribution of each of the two monomeric VHHs to the binding characteristics of the resulting biparatopic constructs, and hence superiority of the biparatopic design over monomeric VHHs. In particular the synergy is apparent for binding molecules targeting the IGF2R, such as binding molecules comprising VHH domains 13E8 and 13F11.

### EXAMPLES

### Example 1: (Over-) Expression of PDGFRB and/or IGF2R in Various Clinical and Experimental Fibrotic Afflictions

Literature search provided ample evidence confirming the (over-) expression of PDGFRB and/or IGF2R by putative fibrogenic effector cells in various clinical and experimental fibrotic afflictions.

### 1.1: Over-Expression of PDGFRB and IGF2R in Human Liver Fibrosis.

For over-expression of PDGFRB in liver fibrosis see Lambrecht et al (EBioMedicine, 2019; 43:501-512. Over-expression of IGF2R in experimental Liver Fibrosis has been demonstrated by Van Beuge et al, (Pharm. Res., 2011; 28:2045-54). IGF2R over expression in fibrotic rats has been demonstrated by Greupink et al (Pharm. Res., 2006; 23:1827-34).

### 1.2: Over-Expression of PDGFRB in Human Glomerular and Interstitial Renal Fibrosis.

Over-expression of PDGFRB in human renal fibrosis has been demonstrated by Boor et al (EMBO Mol. Med., 2020; 12: e11021).

### 1.3. Over-Expression of PDGFRB in Lung Fibrosis.

Over-expression of PDGFRB in idiopathic and secondary pulmonary hypertension has been demonstrated by Overbeek et al (Arthritis Res. Ther. 2011; 13: R61). Over-expression of PDGFRB in human idiopathic pulmonary fibrosis has been demonstrated by Yamaguchi et al (Biochem. Biophys. Res. Commun. 2020; 528:269-275).

### 1.4. Over-Expression of PDGFRB in Systemic Sclerosis/Scleroderma.

Over-expression of PDGFRB in scleroderma/systemic sclerosis has been demonstrated by Klareskog et al (Arthritis Rheum. 1990; 33:1534-41).

### 1.5. Over-Expression of PDGFRB in Inflammatory Bowel Disease (IBD)

Over-expression of PDGFRB has been demonstrated by Yamaguchi et al, (Tohoku J. Exp. Med. 2001; 195:21-33).

### 1.6. Over-Expression of PDGFRB in Stroma of Human Malignant Tumors.

Over-expression of PDGFRB has been demonstrated by Paulsson et al, (Am. J. Pathol. 2009; 175:334-341).

### Example 2: Selection of Human Antibody Fragments Specific for IGF2R or PDGFRB

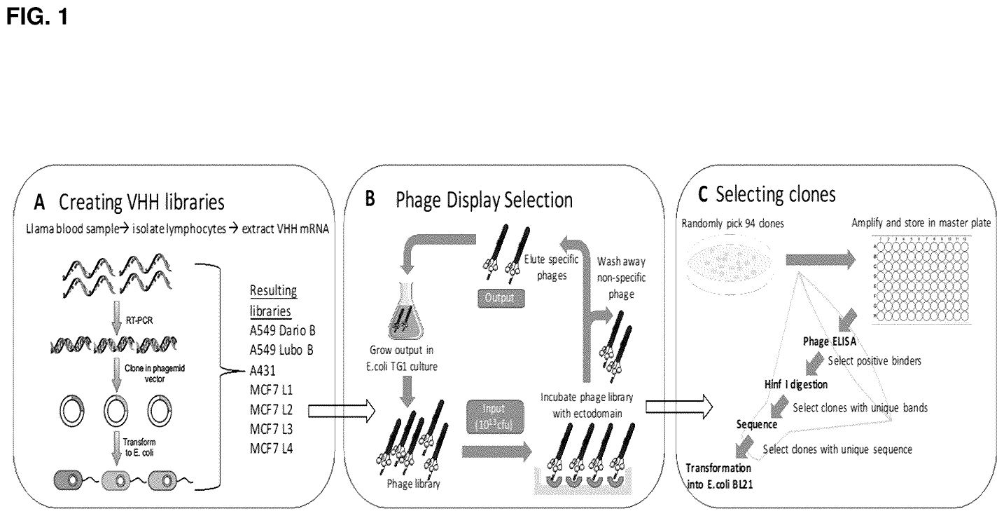

Llama immunization, cell collection, phage VHH selections and the constitution of libraries. VHH selection based on target affinity and HSC internalization: IGF2R (rat and human). VHH selection based on target affinity and HSC internalization: IGF2R (rat and human). A general procedure for generating and characterizing antigen-specific VHHs is depicted in FIG. 1.

### 2.1. IGF2R

Our aim was to obtain VHHs that bind specifically and with high affinity to insulin-like growth factor 2 receptor (IGF2R). The starting material was a previously generated Llama VHH cDNA phagemid library. The library consisted of a large pool of phagemids containing VHH sequences extracted from peripheral blood mononuclear cells of llamas immunized with human A549 cells which endogenously express human IGF2R (hIGF2R) (as in FIG. 1A). Library size, as assessed by means of colony titrations upon DNA transformation was approximately 108 colony forming units (cfu).

Phages were prepared from the VHH cDNA library and the phase library was subjected to two subsequent enrichment (selection-) rounds (as in FIG. 1B). As the selection substrate, immobilized recombinant human extracellular domain of IGF2R (hIGF2R-ECD) was used. In each round, approximately 1011 colony forming units (cfu) of phages were incubated on different concentrations of hIGF2R-ECD, eluted, amplified and then used for the next round of selections. The phage output after the second selection round was clearly higher than in the first round, at 1.2×10⁹ and 3.3×10⁷, respectively, demonstrating that VHH binders had been enriched. After the selection rounds (FIG. 1B), clones are selected (FIG. 1C).

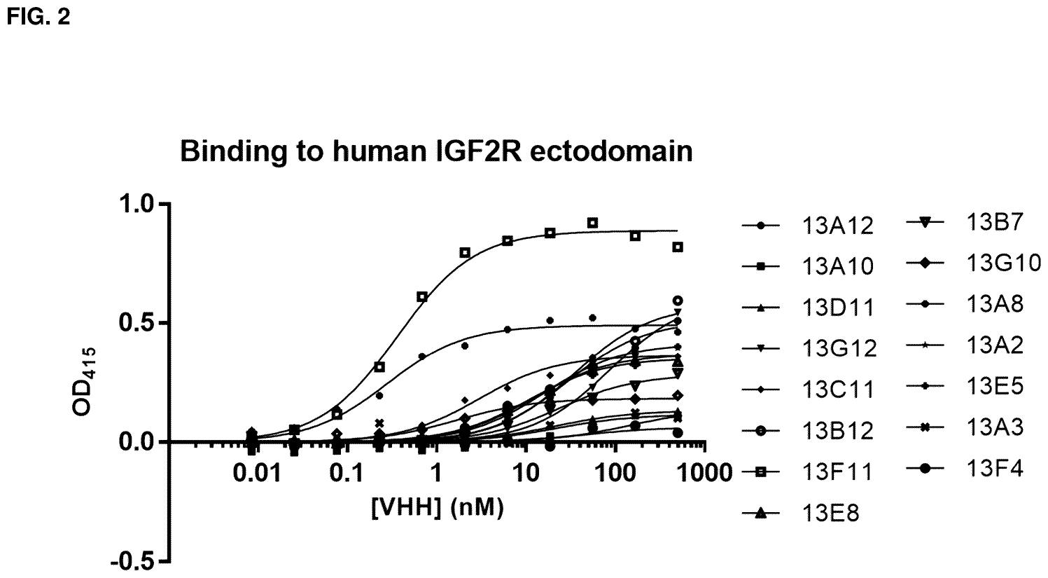

After the second selection round, 94 phage clones were randomly picked and assessed for binding to hIGF2R-ECD in a phage ELISA. Clones showing affinity were analyzed by restriction enzyme analysis using Hinfl and then categorized according to their restriction pattern. From each category, multiple clones were selected, and the amino acid sequences of these binders were identified (Table 2). Finally this resulted in a panel of 15 unique hIGF2R binders (FIG. 2).

### 2.2 PDGFRB

Our aim was to obtain VHHs that bind specifically and with high affinity to rat- and human platelet-derived growth factor receptor beta (rPDGFRB and hPDGFRB, respectively). To obtain VHH against hPDGFRB, two llamas (SNL152 and SNL153) were immunized with recombinant hPDGFRB ectodomain (hPDGFRB-ECD) and murine SCC VII cells which transgenically expressed the full-length human PDGFRB receptor (SCC-hPDGFRB) Simultaneously, to obtain VHH against rPDGFRB, two llamas (SNL154 and SNL155) were immunized with recombinant rPDGFRB ectodomain (rPDGFRB-ECD) and murine SCC VII cells which transgenically expressed the full-length rat PDGFRB receptor (SCC-rPDGFRB). Immunizations were performed in a five-step immunization scheme (Table 3). It must be noted that llamas SNL154 and SNL155 also received injections of SCC VII cells transgenically expressing rat IGF2R (SCC-rIGF2R), as a means to generate a VHH cDNA library from which additional rIGF2R-binding VHHs might be isolated (complementary to the approach outlined in example 2.1). Eight days after the last injection, blood was collected, peripheral blood lymphocytes (PBLs) were purified, and their RNA was extracted.

TABLE 3
Immunization schedule

| | | Llamas SNL152 and | Llamas SNL154 and |
|---|---|---|---|
| Injection | Day | SNL153 | SNL155 |
| 1 | 0 | 25 μg hPDGFRB ECD | 25 μg rPDGFRB ECD and |
| | | | SCC-rIGF2R |
| 2 | 14 | 25 μg hPDGFRB ECD | 25 μg rPDGFRB ECD and |
| | | | SCC-rIGF2R |
| 3 | 28 | 25 μg hPDGFRB ECD | 25 μg rPDGFRB ECD and |
| | | | SCC-rIGF2R |
| 4 | 43 | 25 μg hPDGFRB ECD | 25 μg rPDGFRB ECD and |
| | | | SCC-rIGF2R |
| 5 | 70 | SCC-hPDGFRB | SCC-rPDGFRB and |
| | | | SCC-rIGF2R |

Per Llama, a VHH cDNA library was created according to the following procedure. The extracted RNA (40 μg, 4 reactions of 10 μg each) was transcribed into cDNA using the Invitrogen reverse transcriptase kit and the cDNA was cleaned with a QIAquick PCR purification kit (Qiagen) both according to manufacturers' protocol. IG H fragments (conventional and heavy chain) were amplified using primers annealing at the leader sequence region and at the CH2 region. The PCR product was loaded on a 1% agarose gel, after which the 700 bp fragment was cut out from the gel and purified with the QIAquick kit (Qiagen) according to manufacturers' protocol. Subsequently, 80 ng of the purified DNA was used as a template for a nested PCR to introduce restriction sites (end volume 800 μL). The amplified fragment was cleaned with a QIAquick PCR purification kit according manufacturers' protocol and eluted in 120 μL elution buffer. The eluted DNA was digested with BstEII and SfiI after which the sample was run on a 1.5% TAE-agarose gel and the 400 bp fragment was isolated from the gel. The DNA fragments were isolated by the QIAquick cleanup kit (Qiagen) according to manufacturers' protocol and eluted in 100 μL elution buffer.

To obtain the phagemid libraries, the cDNA fragments (330 ng) were ligated into a linearized and dephosphorylated phagemid vector (pHEN1 derivative, 1 μg) and ligation product was used to transform TG1 bacterial cells. The numbers of transformants were calculated from dilutions of plated out TG1 cells (8 mL). The titer of the library (Table 4) was calculated by counting colonies in the highest dilution and using the formula below: Library size=(amount of colonies)×(dilution)×8 mL/0.005 mL (spotted volume). The insert frequency was determined by picking 24 different clones from each of the library transformations and running a colony PCR. The insert frequency was 100% for library 1, 2 and 4, and 96% for library 3.

TABLE 4
Calculated library sizes

| Library | 1 | 2 | 3 | 4 |
|---|---|---|---|---|
| Llama | SNL152 | SNL153 | SNL154 | SNL155 |
| Size | 6.4 × 10⁷ | 1.4 × 10⁷ | 9.6 × 10⁶ | 4.8 × 10⁶ |
| OD₆₀₀ | 42.3 | 51.8 | 49.3 | 58.0 |

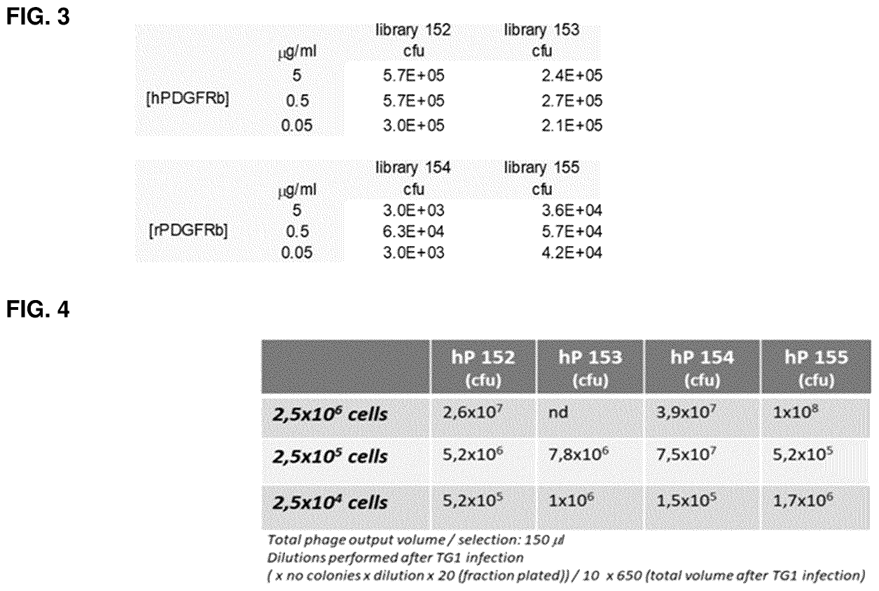

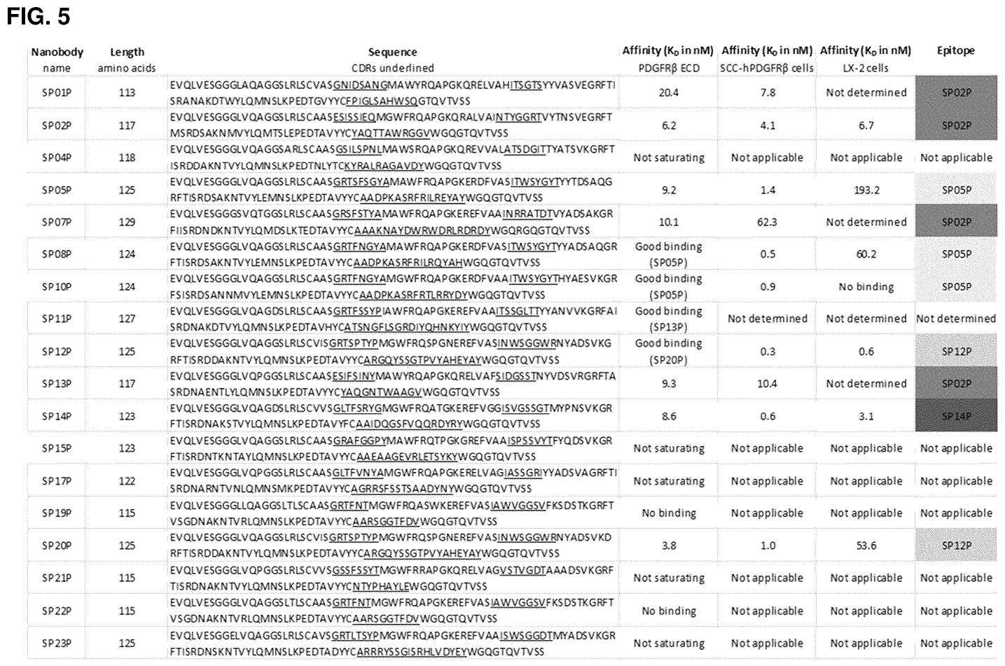

Phages were prepared from the libraries and two enrichment (selection) rounds were executed. In the first selection round, hPDGFRB-ECD and rPDGFRB-ECD were used as the selection substrate for libraries 1&2, and 3&4, respectively). Phages were incubated on different concentration of the substrate, eluted and amplified. The phage outputs of the first selection round are displayed in FIG. 3. In the second selection round, SCC-hPDGFRB cells and SCC-rPDGFRB cells were used as the substrates for libraries 1&2 and 3&4, respectively. In this second step, selection was biased towards internalizing VHH by acid washing the cells to elute bound but not internalized phages. This second selection round was carried out with the output from the first round from the wells with the lowest ECD density. The phage outputs of the second selection round are displayed in FIG. 4.

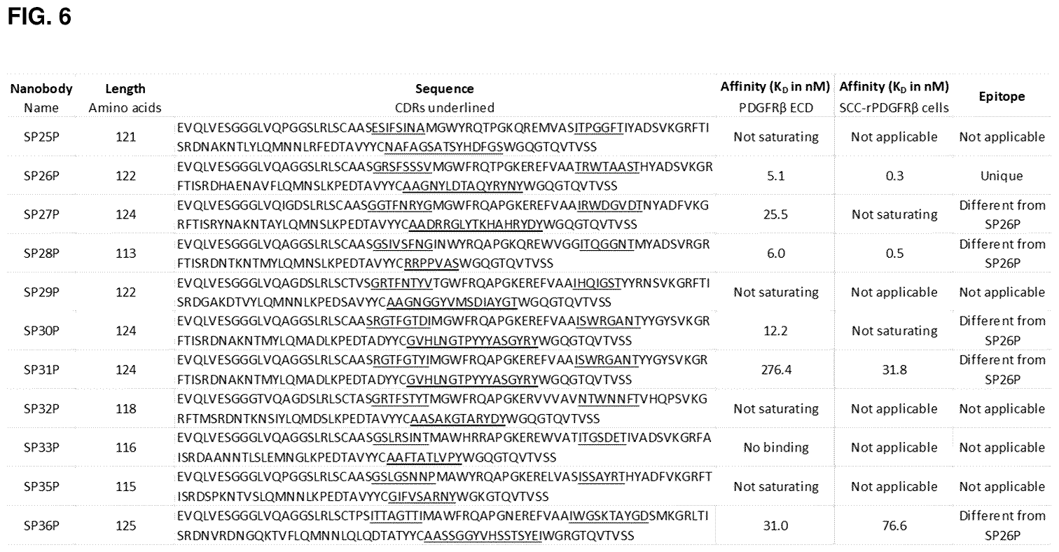

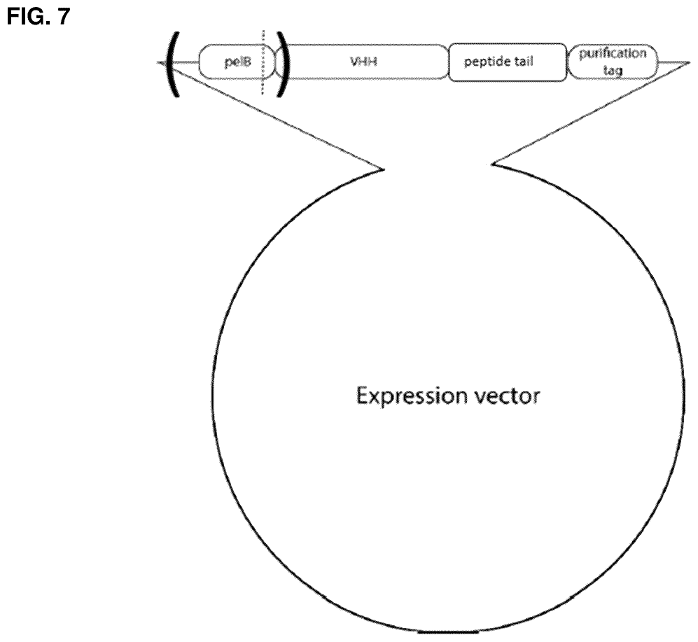

After the second selection round, 94 phage clones were randomly picked and assessed for binding to hPDGFRB-ECD or rPDGFRB-ECD in a phage ELISA. Clones showing high affinity were then further characterized by restriction enzyme analysis using Hinfl and then categorized according to their restriction pattern. One clone from each category was selected, and the amino acid sequences of these binders were identified. Finally, this resulted in 18 unique VHHs from Llamas 152 and 153 immunized with hPDGFRB (FIG. 5), and 11 unique VHHs from Llamas 154 and 155 immunized with rPDGFRB (FIG. 6).

### Example 3: Design of Genes for Production of VHHs and VHH Constructs

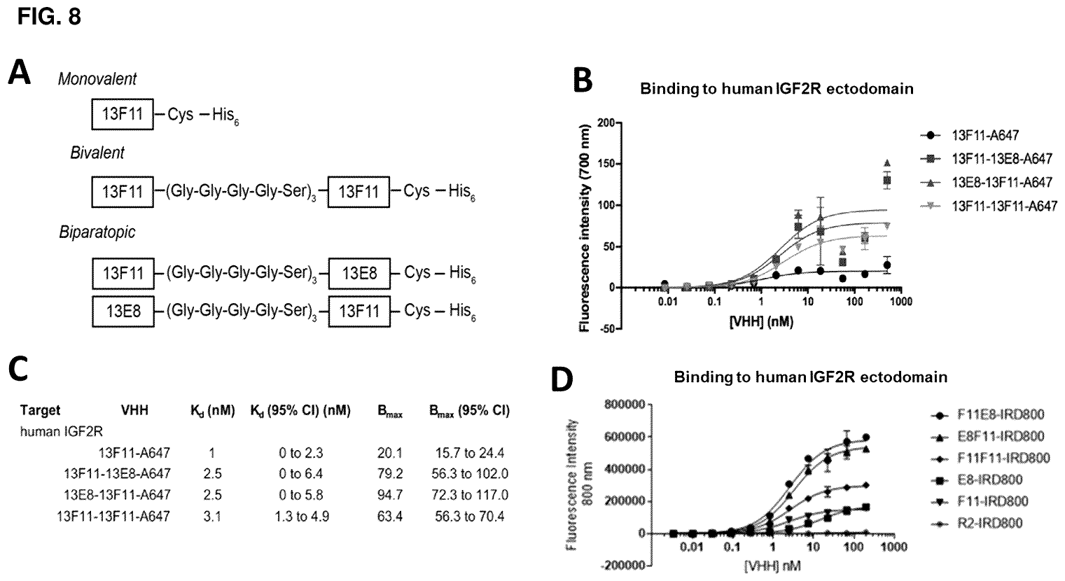

To produce monomeric VHHs and VHH constructs in bacterial cells, genes of the selected VHHs were cloned into a pET-21 or pET-28 vector (FIG. 7). This vector contains the PelB sequence which directs the transport of the produced protein in the bacterial cells to the periplasm, a T7 promotor and terminator, and in between a lac operon which allows induction of the VHH protein upon addition of Isopropyl β-D-1-thiogalactopyranoside (IPTG), an ampicillin/kanamycin resistance gene for selection of transformed cells. Part of the expression cassette is a sequence encoding for one free C-terminal cysteine, which ultimately serves as the conjugation handle in the produced proteins, and a C-terminal Histidine, EPEA or other affinity purification tag with a thrombin cleavage site to remove the tag when desired. For production in mammalian cells, a similar insert in an appropriate mammalian expression vector is used.

### Example 4: The Production of Monomeric, Dimeric, and Multimeric Binding Molecules Comprising VHH Domains

E. coli BL21-DE3 Codonplus (Stratagene) were heat-shock transformed with VHH-encoding plasmid DNA (example 3) and grown in the presence of antibiotics for selection of transformed cells. A single colony was picked and used to inoculate 10 mL 2×YT (supplemented with 2% (w/v) glucose, 35 μg/mL chloramphenicol and 100 μg/mL ampicillin or 30 μg/mL kanamycin) and the inoculated medium was incubated overnight at 37° C. and 180 rpm. Subsequently, the overnight culture was diluted 1/100 in 900 mL Terrific Broth (supplemented with 100 mL KPO buffer, 0.1% (w/v) glucose and 100 μg/mL ampicillin or 30 μg/mL kanamycin) and the culture was incubated at 37° C. and 180 rpm until OD₆₀₀ reached values between 0.5 and 0.8, after which 1 mL of 1 M IPTG was added to the culture to induce protein production and incubation was continued overnight at 25° C. and 180 rpm. The bacterial cells were harvested by spinning down the suspension culture at 4700 rpm for 15 min at 4° C. followed by resuspension of the bacterial cells in PBS (30 mL PBS per 800 mL bacterial culture). The periplasma (containing the VHH) was released from the cells by freeze-thawing the suspension twice and harvesting the supernatant after centrifugation at 4° C. and 4700 rpm. The VHHs were purified from the periplasmic fraction with immobilized metal affinity chromatography (IMAC) purification on Talon metal affinity resin (Clontech) (0.75 mL per L bacterial culture). The resin was prewashed with PBS 3 times after which the resin was added to the periplasm and incubated for 30 min at 4° C. and 15 rpm. The resin was separated from the periplasm by spinning down the suspension for 3 minutes at 4° C. and 900 rpm and washed with 0.05% (v/v) TWEEN20 in PBS, followed by 2 washing steps with PBS. Subsequently, the resin was added to Poly-Prep Chromatography Columns (Bio-Rad) followed by pre-elution of non-specifically bound protein with 1 mL of a 15 mM imidazole solution in PBS after which the protein of interest was eluted with a 150 mM imidazole solution in PBS. The protein concentration of the collected fractions were measured on a NanoDrop 1000 spectrophotometer (ThermoFisher Scientific) at 280 nm (set at 1 Abs=1 mg/mL) and the fractions containing the VHH were pooled and dialyzed against a solution of 1 mM TCEP-HCl in PBS with 3.5 kDa MWCO SnakeSkin Dialysis tubing (ThermoFisher Scientific) overnight at 4° C. Purified protein was aliquoted and stored at −20° C. All clones were successfully purified as seen by a clear singular band around 15 kDa on SDS-PAGE.

### Example 5: Characterization of Monomeric VHH Domains

### 5.1 IGF2R

The 15 unique hIGF2R-binding VHH (Example 2.1, FIG. 2) were produced and purified as described in examples 3 and 4. The apparent affinities were determined through binding assay on immobilized hIGF2R-ECD. Briefly, three-fold serial dilution series, starting from 1 μM, of each VHH were plated onto hIGF2R-ECD coated plates. VHH were allowed to bind for two hours after which plates were washed. Then, bound VHH was detected using a VHH-specific IgG antibody, followed by an IRD800CW-conjugated IgG-specific antibody. Plates were read out by fluorimetry. Exemplary results of the binding assays are displayed in FIG. 2.

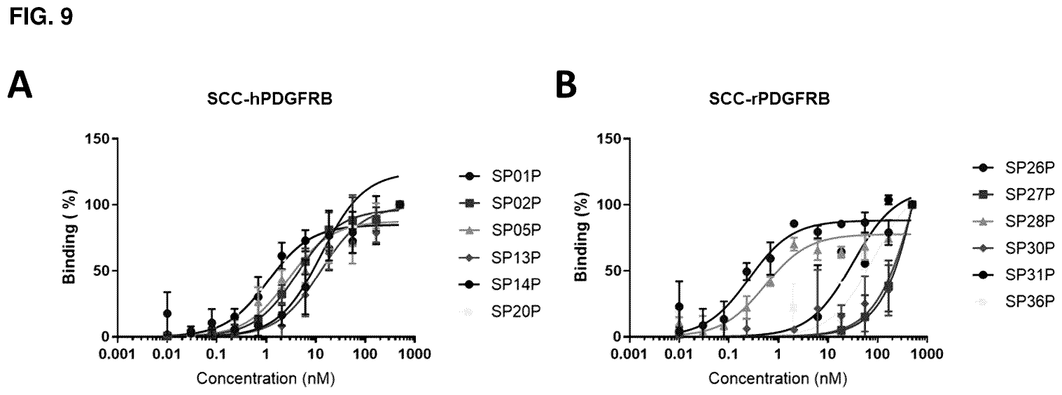

To evaluate functionality of the C-terminal conjugation handle, the VHH 13F11, the bivalent VHH construct 13F11-13F11, and the biparatopic VHH constructs 13F11-13E8 and 13E8-13F11 (FIG. 8A) were conjugated to the fluorophores Alexa Fluor 647 or IRDye 800 using maleimide chemistry (see example 10.2), in order to produce fluorescent conjugates. These conjugates were then subjected to direct binding assays on immobilized hIGF2R-ECD, meaning that plates were read out immediately after washing, without additional detection steps. As shown in FIGS. 8B and 8C, the resulting binding curves demonstrated retained affinity. For detailed analysis, binding parameters K_{D} and Bₘₐₓ were calculated in Graphpad (version 8.3), using one-site specific binding analysis (FIG. 8D). Similar affinities were observed as compared to those of non-conjugated VHH with, e.g., the K_{D} of 13F11-A647 returning at 1.00±0.61 nM (K_{D}±SEM, n=2), which is slightly but not significantly higher than the K_{D} of native 13F11 (0.23±0.05 nM). This demonstrated that site-directed C-terminal conjugation does not significantly alter affinity.

### 5.2 PDGFRB

The 18 unique hPDGFRB binders from libraries 1 and 2, and the 11 unique rPDGFRB binders from libraries 3 and 4 (Example 2.2, FIGS. 5 and 6) were produced and purified as described in examples 3 and 4. Then, the apparent affinities were determined with binding assays on hPDGFRB-ECD and rPDGFRB-ECD, respectively, the setup described in example 5.1. Results are summarized in FIGS. 5 and 6 for the hPDGFRB and rPDGFRB VHHs, respectively.

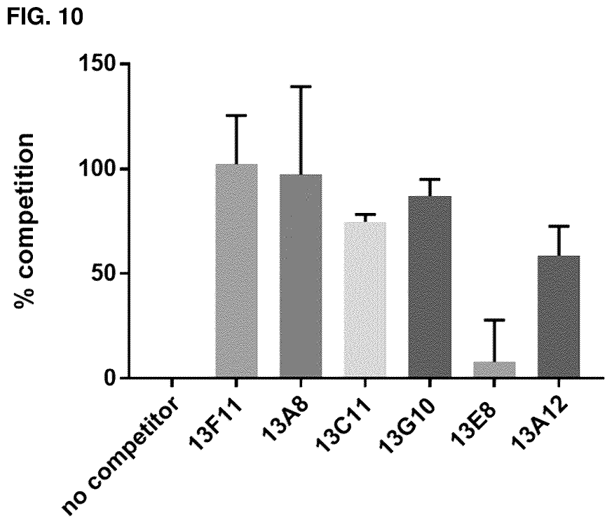

High affine binders to hPDGFRB-ECD or rPDGFRB-ECD were subsequently assessed for binding to SCC VII cells transfected with hPDGFRB and rPDGFRB, respectively. As shown in FIG. 9, the VHHs obtained from libraries 1 and 2 displayed affinity to SCC-hPDGFRB cells, and VHHs obtained from libraries 3 and 4 displayed good binding to SCC-rPDGFRB cells.

### Example 6: Determination of VHHs Binding to Non-Overlapping Receptor Epitopes to Allow Construction of Biparatopic VHH Constructs

### 6.1 IGF2R

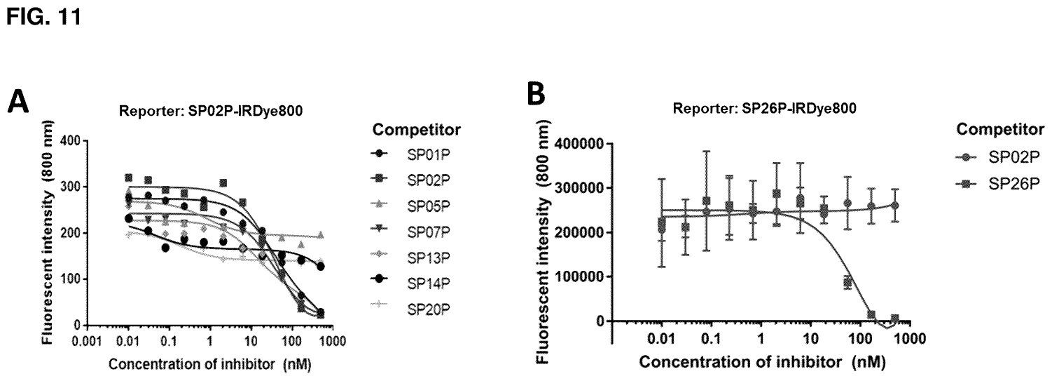

Biparatopic VHH constructs contain two VHH which bind to a different epitope, i.e. which are non-competitive binders. To identify (a) pair(s) of non-competitive binders, competition assays were performed. In the example shown, an Alexa fluor-conjugate of the high affine VHH 13F11 was used as the reporter VHH (13F11-A647, see example 10.2). In the assay, a non-saturating amount (10 nM) of 13F11-A647 was incubated with immobilized hIGF2R-ECD in the presence or absence of 250 nM unconjugated competitor VHH to allow competition to take place. As shown in FIG. 10, unconjugated 13F11 competed with 13F11-A647 demonstrating assay validity. Of the VHHs tested, 13A8, 13A12, 13C11, and 13G10 competed with 13F11 demonstrating that they bound overlapping epitopes, and 13E8 did not compete with 13F11 demonstrating that it bound to a non-overlapping epitope.

### 6.2 PDGFRB

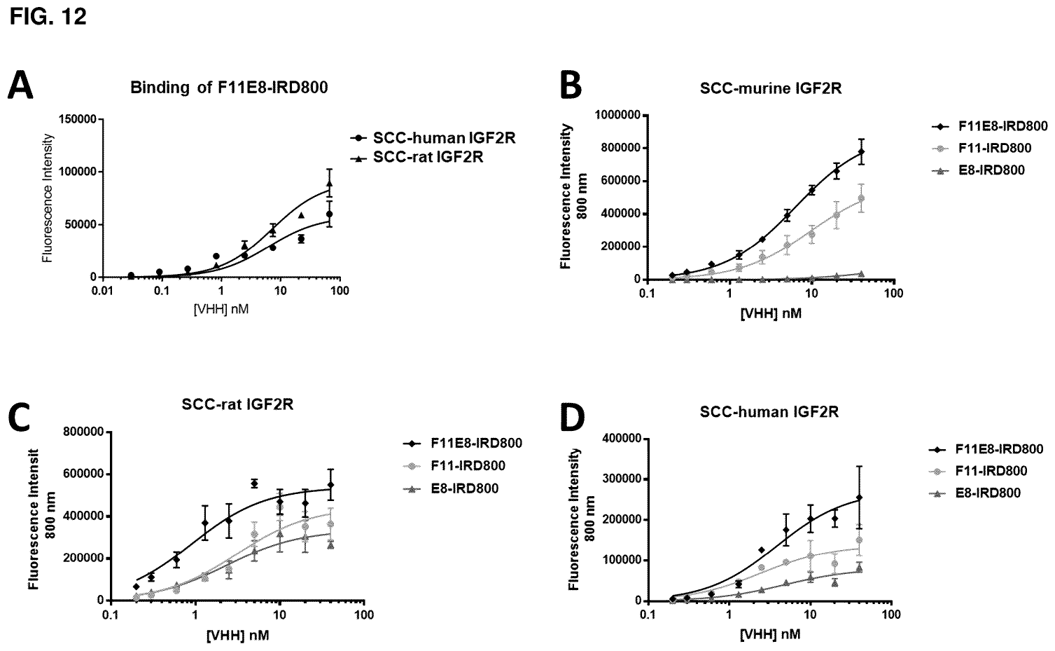

Similar as to in example 6.1, competition assays were performed to identify unique hPDGFRB- or rPDGFRB binders. Across 10 PDGFRB-binding VHHs obtained from libraries 1 and 2 (e.g. the VHH raised against hPDGFRB), four main groups were identified being first SP01P, SP02P, SP07P and SP13P, second SP05P, SP08P and SP10P, third SP12P and SP20P and finally SP14P. All VHH within each of the first three groups mutually competed. Across the three groups, and the lone VHH SP14P, no competition was observed demonstrating that VHH had been raised against at least four different epitopes of the hPDGFRB: those of SP02P, SP05P, SP12P, and SP14P (FIG. 5 rightmost column, exemplary binding curves using SP02P as the reporter VHH are shown in FIG. 11A). Similarly, across the VHH obtained from libraries 3 and 4 (e.g. the VHH raised against rPDGFRB), SP26P showed to have a unique epitope compared to SP27P, SP28P, SP30P, SP31P and SP36P (FIG. 6).

To evaluate whether the obtained VHHs would qualify as building blocks for multivalent VHH constructs that bind hPDGFRB as well as rPDGFRB (e.g. ‘species cross-reactive’ VHH constructs), competition assays were performed using the most promising VHHs SP02 (raised against hPDGFRB) and SP26P (raised against rPDGFRB). Competition assay showed that these two VHH did not compete and hence bound to non-overlapping epitopes (FIG. 11B).

### Example 7: Construction and Characterization of Biparatopic VHHs

The design of biparatopic constructs was led by the following critical traits: First, the constructs must be based on VHH with non-overlapping epitopes (the biparatopic design). Second, the constructs must bind with similar affinity to human- and rodent ligands as this would result in constructs suitable for preclinical as well as clinical pharmaceutical development. Finally, the constructs must display a meaningful in vivo plasma half-life in order to ensure meaningful in vivo exposure.

### 7.1 IGF2R

Based on their high affinity and binding to non-overlapping receptor epitopes, VHHs 13F11 and 13E8 were selected for the design of biparatopic and bivalent VHH constructs. Two VHHs were fused with a flexible linker, consisting of three Gly-Gly-Gly-Gly-Ser repeats (FIG. 8A). The sequences were codon optimized for expression in E. coli and obtained as synthesized DNA fragments from GeneArt. The DNA fragments encoding VHHs were cloned into a vector pET28-based vector introducing a C-terminal free cysteine for site-directed conjugation and His6 tag for IMAC purification (FIG. 8A). The VHH constructs were produced and purified as described in example 4. The VHH constructs were conjugated to Alexa Fluor 647 (conjugation method as described in example 10.2) and binding assays were performed on hIGF2R-ECD (as described in example 5.1) to determine the K_{D} and Bₘₐₓ (calculated in Graphpad (version 8.3), using one-site specific binding analysis) (FIG. 8C). The calculated affinities for 13F11, 13F11-13F11, 13F11-13E8 and 13E8-13F11 were 1.00±0.61 nM (K_{D}±SEM, n=2), 3.10±0.87 nM, 2.5±1.87 nM and 2.5±1.56 nM (K_{D}±SEM, n=2), respectively (FIG. 8C). In a similar binding assay, IR dye 800 (IRD800) conjugates were prepared (as outlined in example 10.2) of the monomeric VHHs 13F11 and 13E8, the biparatopic VHH construct 13F11-13E8, the reciprocal biparatopic construct 13E8-13F11, the bivalent VHH construct 13F11-13F11, and the non-binding (control-) VHH R2. Conjugated constructs were then subjected to binding assay as described above. The biparatopic VHH constructs displayed superior binding characteristics over the monomeric VHHs as well as over the bivalent construct, further demonstrating the superiority of the biparatopic constructs with respect to IGF2R binding (FIG. 8D).

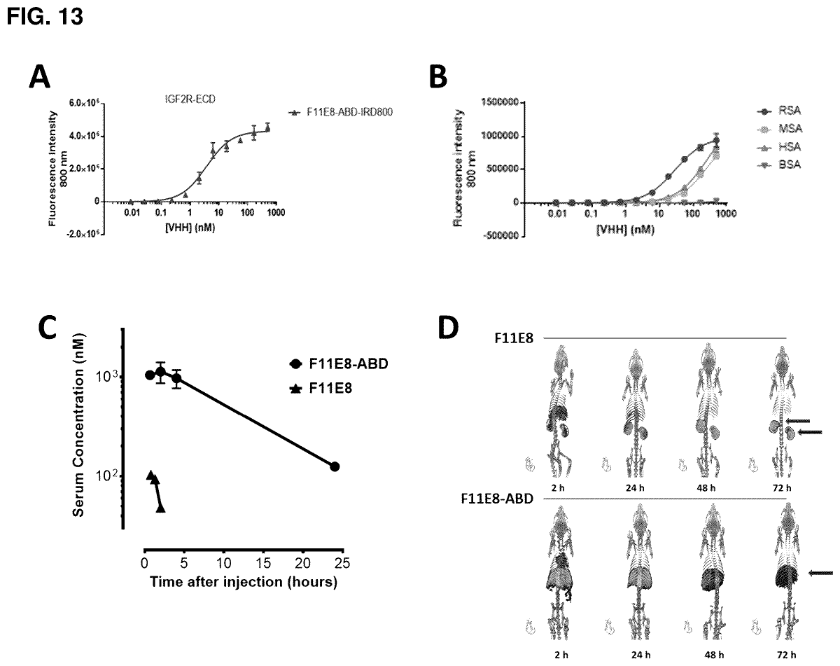

To examine whether 13F11-13E8 is cross-reactive against rodent IGF2R, the construct as well as its corresponding monomeric VHHs were conjugated to IRDye800CW (F11E8-IRD800, conjugation method as described in example 10.2) and the binding affinity was determined using a binding assay on SCC cells that transgenically expressed either murine IGF2R (mIGF2R), rIGF2R, or hIGF2R (FIGS. 12A-D). Clearly, F11E8-IRD800 bound to all three receptors with comparable affinity (<10 nM) confirming species cross-reactivity. This high affinity existed despite of the lower and variable binding affinities of the corresponding monomeric VHHs to the respective substrates, demonstrating the synergistic contribution of the monomeric VHH to the binding characteristics of the resulting biparatopic constructs, and hence the superiority of the biparatopic design over monomeric VHHs. Binding of IRDye800-conjugated 13F11-13E8 (F11E8) to SCC cells transgenically expressing hIGF2R or hIGF2R is displayed in FIG. 12A. FIG. 12A-C shows the binding of IRDye800-conjugated 13F11-13E8 (F11E8) and IRDye800-conjugates of its corresponding monomeric VHHs F11 and E8, to SCC cells transgenically expressing murine-(FIG. 12B), human-(FIG. 12D) or rat (FIG. 12C) IGF2R.

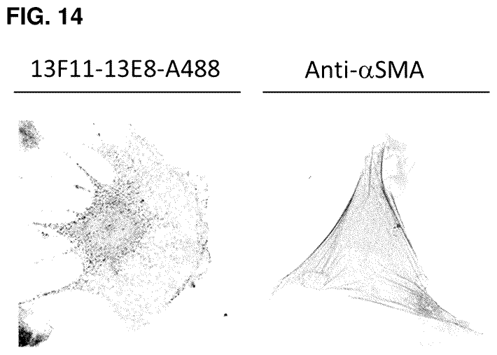

Small proteinaceous molecules such as VHHs or dimeric VHH constructs may be swiftly renally excreted due to their size. This reduces their serum half-life and thus reduces systemic exposure which is a desirable trait in the context of the invention. To confer a meaningful in vivo serum half-life to the construct, 13F11-13E8 was genetically re-engineered to include an in vivo half-life extender in the form of an albumin binding domain (ABD) as described by Johansson et al (Structure, specificity, and mode of interaction for bacterial albumin-binding modules, J. Biol. Chem. 2002 (277): 8114-8120) and subsequently produced and purified as described in Example 4. Subsequently, 13F11-13E8-ABD was conjugated to IRDye800CW (F11E8-ABD-IRD800, conjugation method as described in example 10.2) and the binding affinities of the biparatopic VHH as well as the ABD unit was determined. A low nanomolar binding affinity to hIGF2R-ECD was found for F11-E8-ABD-IRD800 indicating that addition of the ABD domain has no effect on binding of the biparatopic F11-E8 to hIGF2R (FIG. 13A). The binding affinity (K_{D}) of F11-E8-ABD-IRD800 to rat serum albumin (RSA) was 30 nM and to mouse serum albumin (MSA) and human serum albumin (HSA) the binding affinity was approximately 200 nM. No binding was observed to bovine serum albumin (BSA), in line with what is known about this ABD (FIG. 13B).

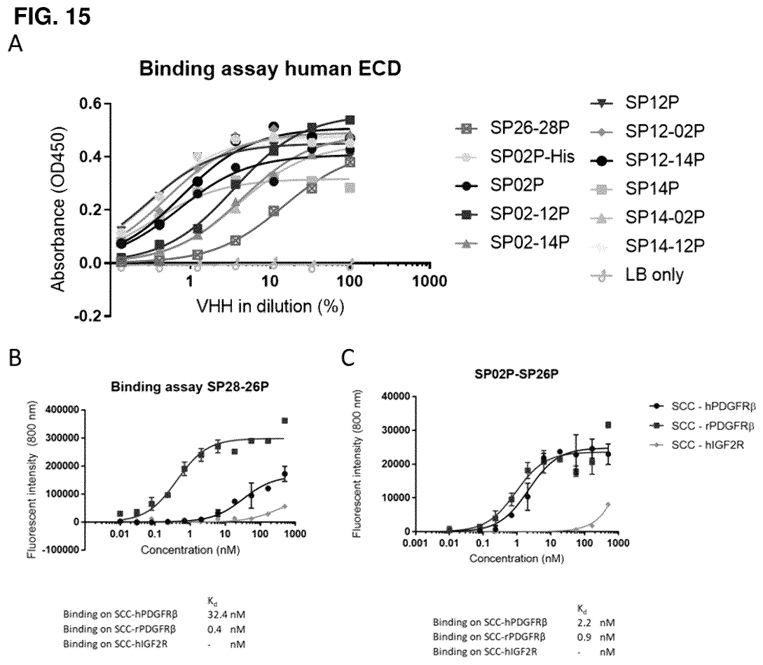

The cross-reactivity of F11-E8-ABD was further evaluated in binding experiments to primary activated rat hepatic stellate cells (HSCs). For this, the construct was conjugated to Alexa Fluor 488 according to the methods described in example 10.2. Activated HSCs were obtained from a perfused liver of a cirrhotic bile duct ligated (BDL) rat through density gradient centrifugation. Cells were kept in culture for 6 days to allow complete activation. As shown in FIG. 14, activated state of the cells was evident from the presence of stress fibers positively stained by aSMA. Simultaneously, binding of the VHH construct to the activated cells was evident, further confirming species cross-reactivity of the construct.

### 7.2 PDGFRB

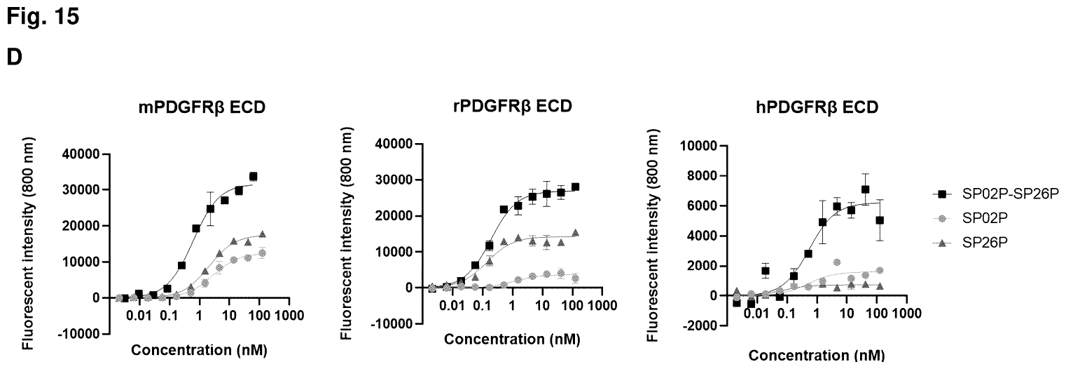

Based on their high affinity and binding to non-overlapping hPDGRB epitopes, various biparatopic constructs were prepared based on VHHs SP02P, SP12P SP14P, SP26P and SP28P. The constructs were produced as described in example 7.1. and subjected to binding assay. All constructs based on VHH that had been raised against hPDGFRB (SP02P, SP12P SP14P) showed high affinity for hPDGFRB. The construct SP26P-SP28P, based on VHH raised against rPDGFRB, displayed the lowest affinity for hPDGFRB (FIG. 15A). Contrary, SP26P-SP28P displayed high affinity for rPDGFRB and low affinity for hPDGFRB (FIG. 15B).

In an effort to pursue a species cross-reactive construct, the VHHs SP02P and SP26P were combined in a biparatopic construct, as these were shown to bind different epitopes (FIG. 11B; SP02P does not compete with binding of fluorescently labelled SP26P to cells expressing PDGFRB). As Shown in FIG. 15C), the binding affinity of this construct for both hPDGFRB and rPDGFRB are below 5 nM. Furthermore, whereas binding of the monomeric VHHs SP02P and SP26P varied across murine PDGFRB ectodomain (mPDGFRB-ECD), rPDGFRB-ECD and hPDGFRB-ECD, the biparatopic construct SP02P-SP26P displayed high affinity regardless of the species. These observations make SP02P (or similar VHH) and SP26P (or similar VHH) particularly promising for use in the present invention. FIG. 15D displays binding of IRDye800-conjugated SP02P-SP26P, and corresponding monomeric VHHs SP02P and SP26P, to murine PDGFRB ECD (left panel), rat PDGFRB ECD (middle panel), or human PDGFRB ECD (right panel).

### Example 8: Improving In Vivo Pharmacokinetics in Rats of 13F11-13E8 Through Addition of an Albumin Binding Domain

The in vivo pharmacokinetic properties of 13F11-13E8-ABD (F11E8-ABD; with ABD) and 13F11-13E8 (F11E8; without ABD) were compared in healthy Sprague-Dawley rats. In brief, rats were dosed intravenously with approximately 3 mg/kg body weight and blood samples were taken at different time points and the concentration of the VHH constructs were determined using an ELISA on immobilized hIGF2R-ECD. The peak serum level (Cₘₐₓ) of 13F11-13E8-ABD exceeded 1000 nM as shown in FIG. 13C. With approximately 20 nmoles infused into the animal, and an estimated serum volume of 10 mL, this Cₘₐₓ would correspond to approximately 50% of the infused dose still being in circulation one hour after dosing demonstrating good bioavailability. On the other hand, 13F11-13E8 was rapidly cleared from circulation and could not be detected anymore within two hours after injection (FIG. 13C). To further confirm the functionality of the ABD, biodistribution of F11E8-ABD and F11E8 were assessed by means of Positron Emission Tomography (PET). In brief, the constructs were radio-labelled with the positron emitter ⁸⁹Zr-desferral according to Verel et al (J Nucl Med. 2003; 44:1271-1281), and administered intravenously to rats after which biodistribution was assessed on subsequent time points by whole-body PET. As evident from FIG. 13D, F11E8 swiftly accumulated in the kidneys (upper panels, arrows) illustrating renal excretion. In contrast, the extended serum half-life of F11E8-ABD allowed it to accumulate in the liver (lower panels, arrow).

### Example 9: Synthesis and Analytical Characterization of Lx Semi-Final Complexes and Maleimide Functionalized Moieties with Small Molecule Kinase Inhibitors: Y27632, Pacritinib, Sacubitril (at), Losartan

9.1. Structures of Y27632-Lx (Semi-Final Moieties) SFMs and Y27632 Maleimide Functionalized Moieties that can be Utilized in the Context of the Present Invention.

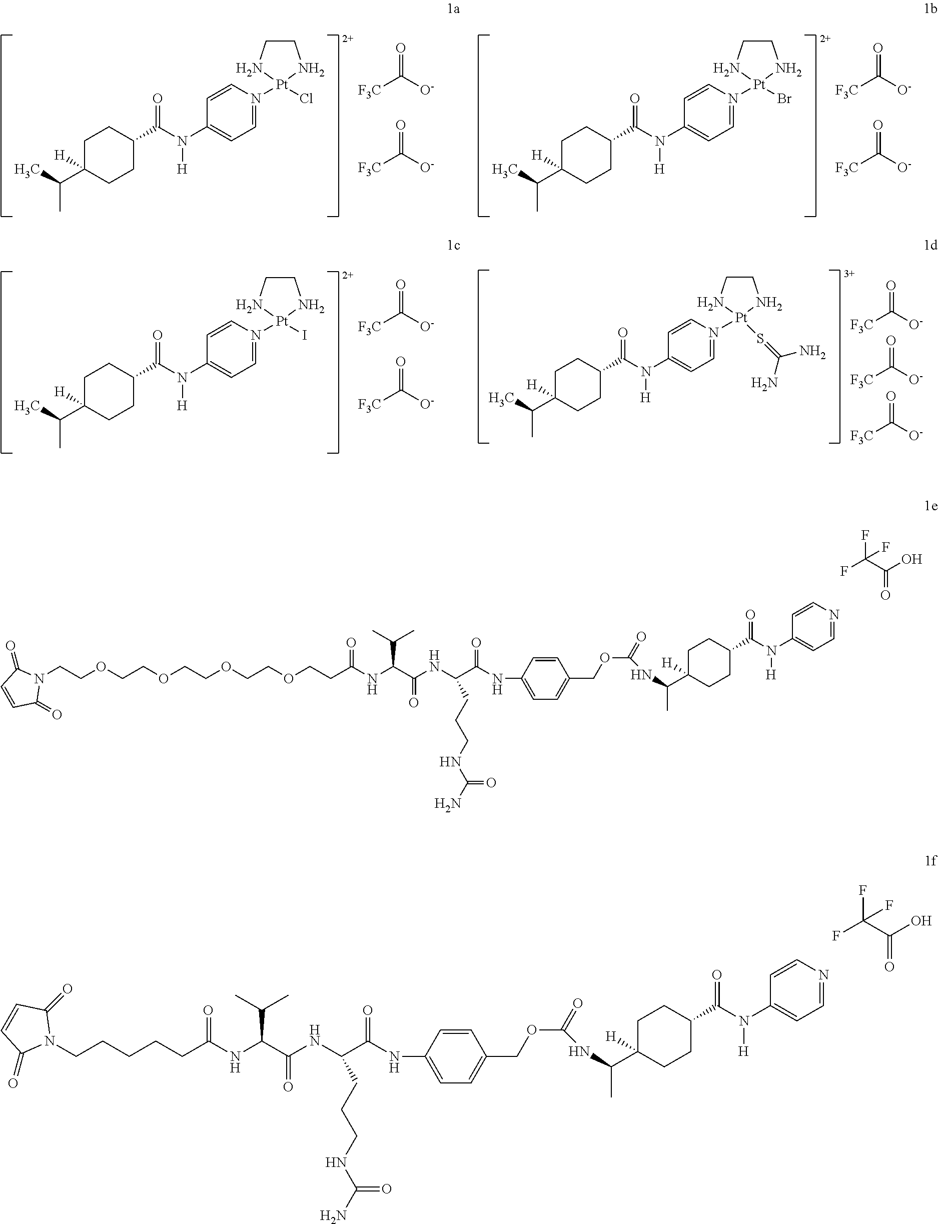

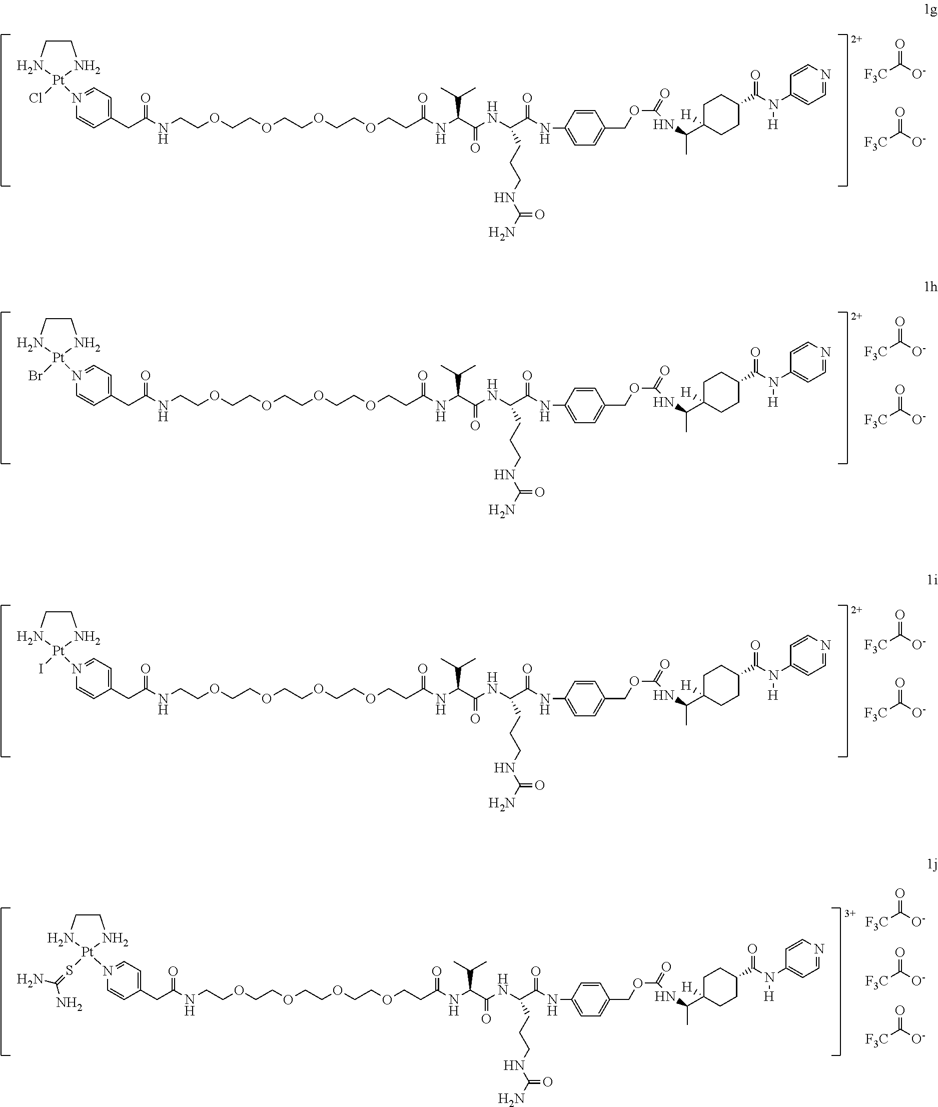

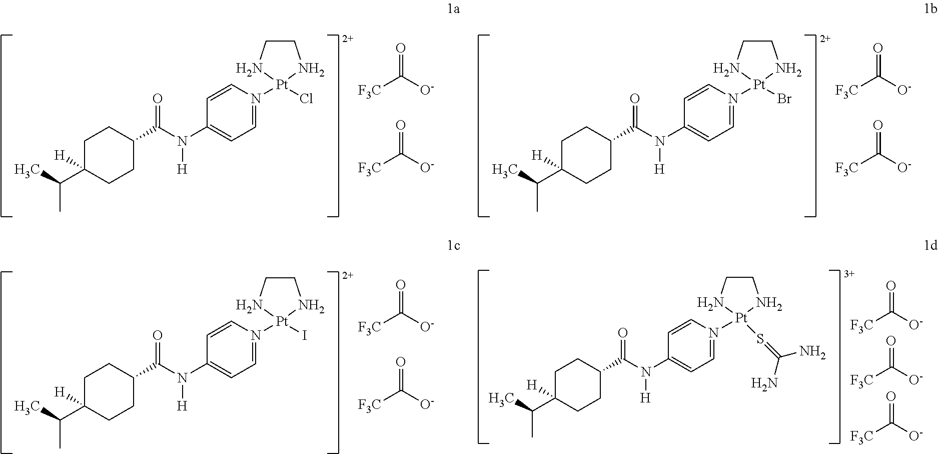

### 9.1.1. Synthesis and Analytical Characterization of Y27632-Lx-Cl (1a)

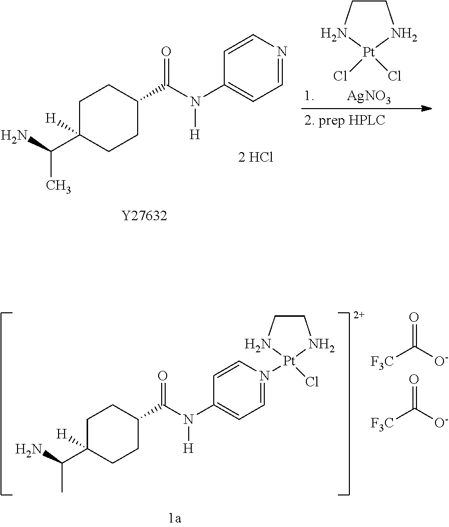

AgNO₃ (85 mg, 500 μmol, 1.0 eq.) was added to a suspension of PtCl₂ (ethane-1,2-diamine) (LxCl₂; 163 mg, 500 μmol, 1.0 eq.) in dry DMF (24.8 mL). The mixture was stirred overnight at room temperature in the dark under argon atmosphere. After that, the suspension was filtered through a 0.2 μm syringe filter to give a 20.2 mM stock solution of activated Pt-complex. Then, to a solution of Y27632×2 HCl (40 mg, 125 μmol, 1.0 eq.) in MilliQ water (14 mL) (pH adjusted to 6.95 using 1 M NaOH), the above prepared 20.2 mM stock solution of the activated Pt-complex (12.4 mL, 250 μmol, 2.0 eq.) was added. The reaction mixture was stirred for 4.5 h at 60° C. in the dark under argon atmosphere. Subsequently, the reaction mixture was filtered through a 0.2 μm filter and 0.9% NaCl was added to the solution (1 mL), after which the solvents were removed under reduced pressure. The residue was dissolved in MilliQ water/MeOH (85:15, 6 mL) and filtered through a 0.2 μm syringe filter. Purification was performed by preparative reverse-phase HPLC (Grace Alltima C18 5 μm column, 22×250 mm; gradient: 15% to 35% MeOH/0.1% TFA in water/0.1% TFA in 36 min.). Product fractions were lyophilized and the product 1a was obtained as a colourless solid (41.8 mg, 43.8% yield).

¹H NMR (400 MHZ, CD₃OD): δ 8.57-8.47 (m, 2H), 7.74-7.68 (m, 2H), 6.09-5.78 (m, 2H), 5.72-5.40 (m, 2H), 3.21-3.11 (m, 1H), 2.80-2.51 (m, 4H), 2.48-2.37 (m, 1H), 2.10-2.01 (m, 2H), 1.98-1.84 (m, 2H), 1.67-1.51 (m, 3H), 1.32-1.15 (m, 5H). 195 Pt NMR (86.0 MHZ, CD₃OD): δ−2512.

HPLC (Grace Alltima C18 5 μm column, 25×4.6 mm) indicated that the product was 95.6% pure (retention time 15.8 min.; gradient: 5% to 25% MeCN/0.1% TFA in water/0.1% TFA in 18 min. measured at a wavelength of 273 nm).

### 9.1.2. Synthesis and Analytical Characterization of Y27632-Lx-I (1c)

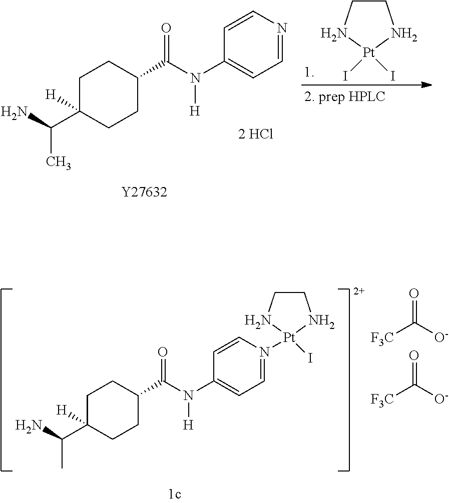

Y27632×2 HCl (10 mg, 31 μmol, 1.0 eq.) in MilliQ water (125 μL) and Pt (ethane-1,2-diamine)I₂ (LxI₂; 14.90 mg, 29 μmol, 0.95 eq.) were dissolved in dry DMF (250 μL) and the reaction mixture was shaken for 48 h at 60° C. The reaction mixture was then diluted with 10 mM NaI in MilliQ/MeOH (1:1, 3 mL) and incubated for 1 h at 25° C. after which the suspension was filtered through a 0.2 μm syringe filter. Purification was performed by preparative reverse-phase HPLC (Grace Alltima C18 5 μm column, 22×250 mm; gradient: 15% to 50% B, whereas eluent A: 95/5 water/MeOH (+0.1% TFA) and eluent B: 5/95 water/MeOH (+0.1% TFA) in 40 min.). Product containing fractions were collected and lyophilized and the product 1c was obtained as a yellow solid (11.0 mg, 41.1% yield).

HRMS (ESI⁺) C₁₆H₂₉N₅OPt [M+H]⁺629.1065, found 629.1092.

HPLC (Grace Alltima C18 5 μm column, 25×4.6 mm) indicated that the product was 99.1% pure (retention time 13.3 min.; gradient: 5% to 50% MeCN/0.1% TFA in water/0.1% TFA in 20 min. measured at a wavelength of 273 nm).

### 9.1.3. Synthesis and Analytical Characterization of Mal-PEG₄-Val-Cit-PAB-Y27632 (1e)

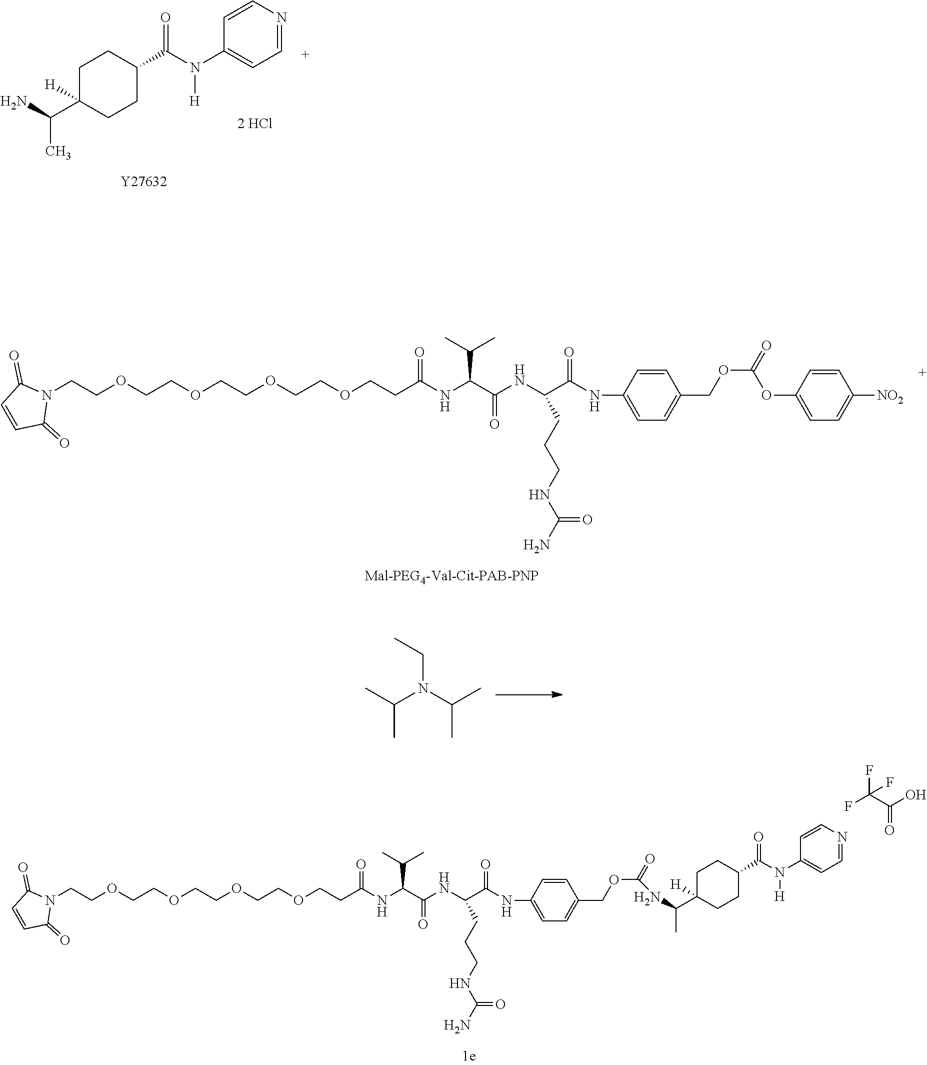

Y27632×2 HCl (18 mg, 56 μmol, 1.0 eq.), Mal-PEG₄-Val-Cit-PAB-PNP (49 mg, 56 μmol, 1 eq.) and triethylamine (19.6 μL, 112 μmol, 2.0 eq.) were dissolved in dry DMSO (1 mL) and the reaction mixture was stirred for 45 min at 25° C. The reaction mixture was then diluted with MilliQ/MeOH (1:1, 3 mL) after which the suspension was filtered through a 0.2 μm syringe filter. Purification was performed by preparative reverse-phase HPLC (Grace Alltima C18 5 μm column, 22×250 mm; gradient: 20% to 40% B, whereas eluent A: 95/5 water/MeCN (+0.1% TFA) and eluent B: 5/95 water/MeCN (+0.1% TFA) in 80 min.). Product containing fractions were collected and lyophilized and the product 1e was obtained as a yellow solid (22.3 mg, 36.3% yield).

HRMS (ESI⁺) C₄₈H₇₀N₉O₁₃ [M+H]⁺980.5088, found 980.5093.

HPLC (Grace Alltima C18 5 μm column, 25×4.6 mm) indicated that the product was 97.1% pure (retention time 10.1 min.; gradient: 20% to 100% MeCN/0.1% TFA in water/0.1% TFA in 20 min. measured at a wavelength of 273 nm).

### 9.1.4. Synthesis and Analytical Characterization of Mal-Val-Cit-PAB-Y27632 (1f)

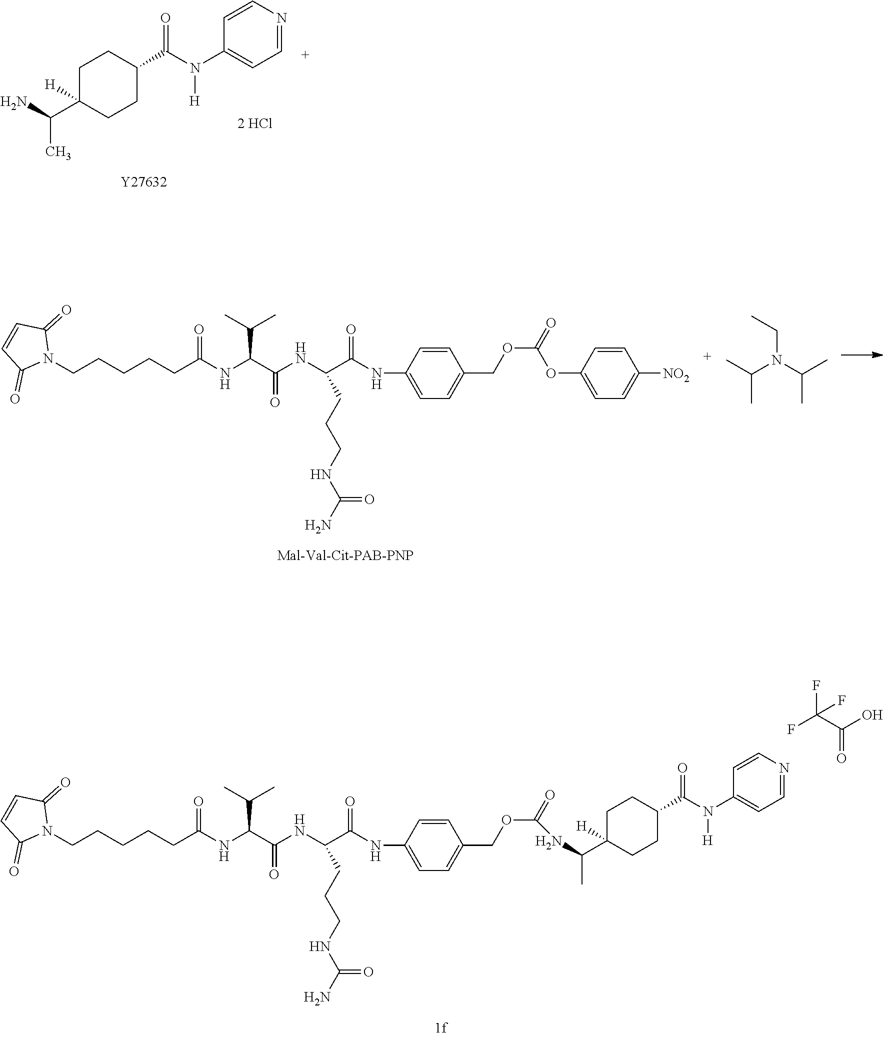

Y27632× 2 HCl (20 mg, 62 μmol, 1.0 eq.), Mal-Val-Cit-PAB-PNP (48 mg, 66 μmol, 1.05 eq.) and triethylamine (22.2 μL, 125 μmol, 2.0 eq.) were dissolved in dry DMSO (1 mL) and the reaction mixture was stirred for 45 min at 25° C. The reaction mixture was then diluted with MilliQ/MeOH (1:1, 3 mL) after which the suspension was filtered through a 0.2 μm syringe filter. Purification was performed by preparative reverse-phase HPLC (Grace Alltima C18 5 μm column, 22×250 mm; gradient: 20% to 40% B, whereas eluent A: 95/5 water/MeCN (+0.1% TFA) and eluent B: 5/95 water/MeCN (+0.1% TFA) in 80 min.). Product containing fractions were collected and lyophilized and the product 1f was obtained as a yellow solid (23.5 mg, 39.2% yield).

HRMS (ESI⁺): C₄₃H₆₀N₉O₉ [M+H]⁺846.4509, found 846.4472.

HPLC (Grace Alltima C18 5 μm column, 25×4.6 mm) indicated that the product was 95.6% pure (retention time 10.5 min.; gradient: 20% to 100% MeCN/0.1% TFA in water/0.1% TFA in 20 min. measured at a wavelength of 273 nm).

9.2. Structures of Sacubitril-Lx SFMs that can be Utilized in the Context of the Present Invention

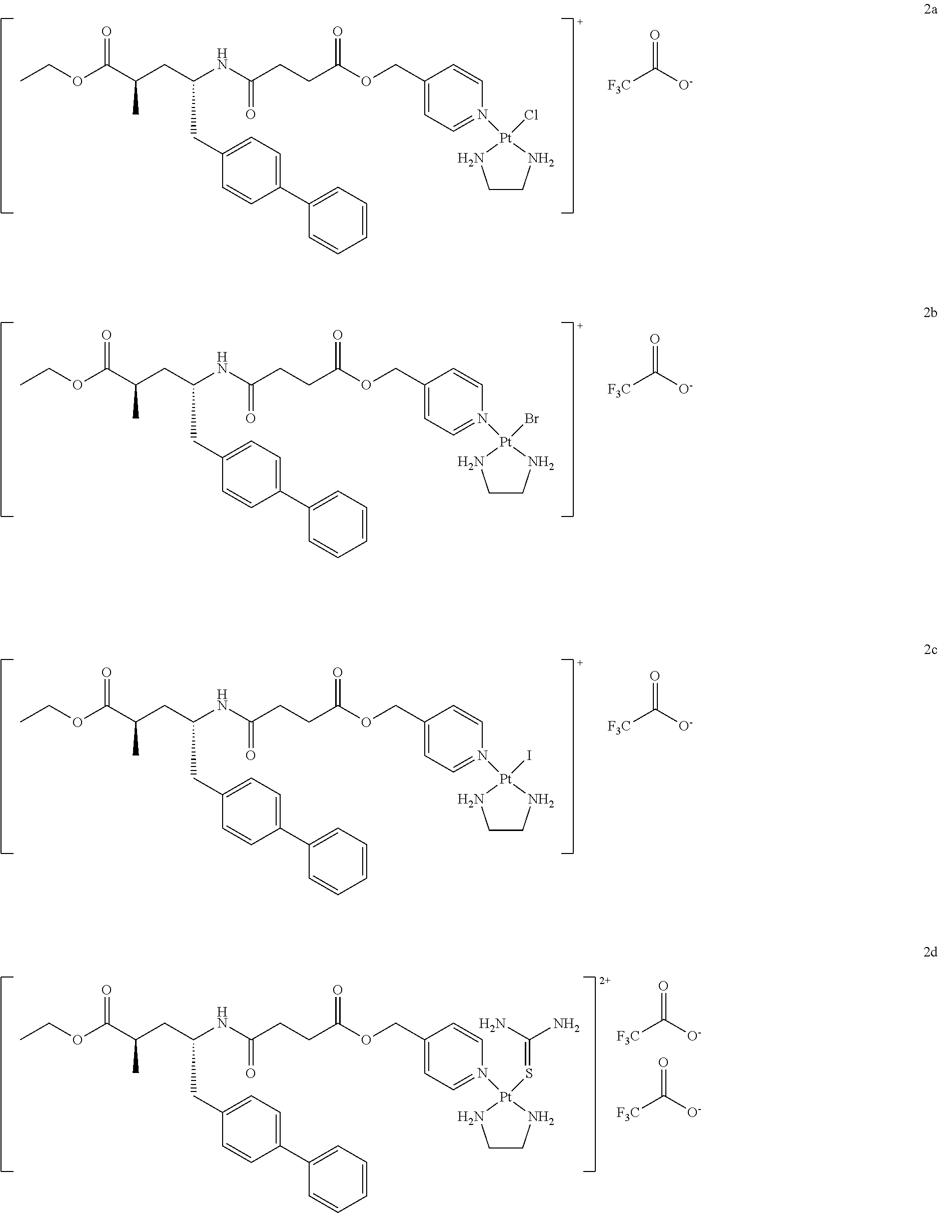

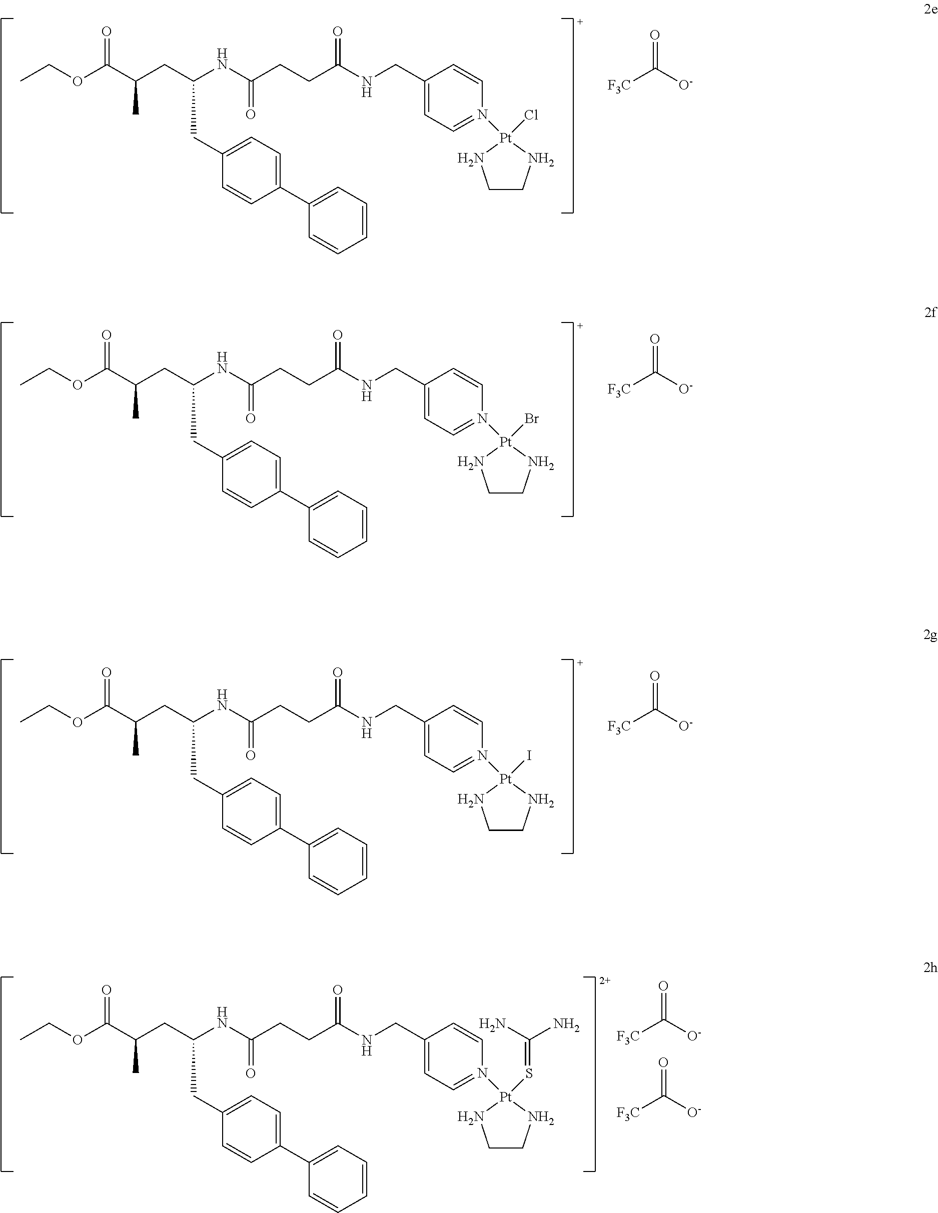

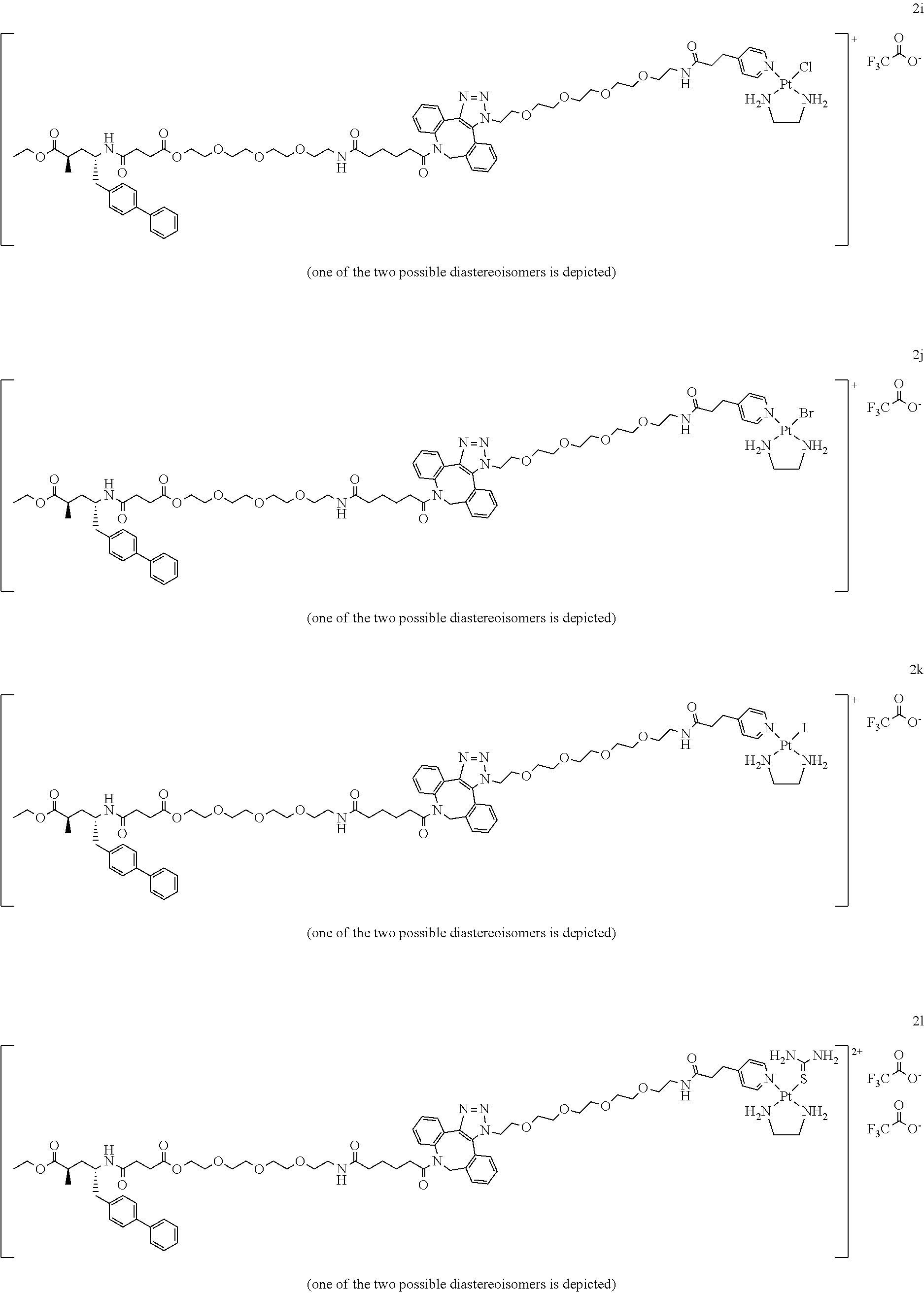

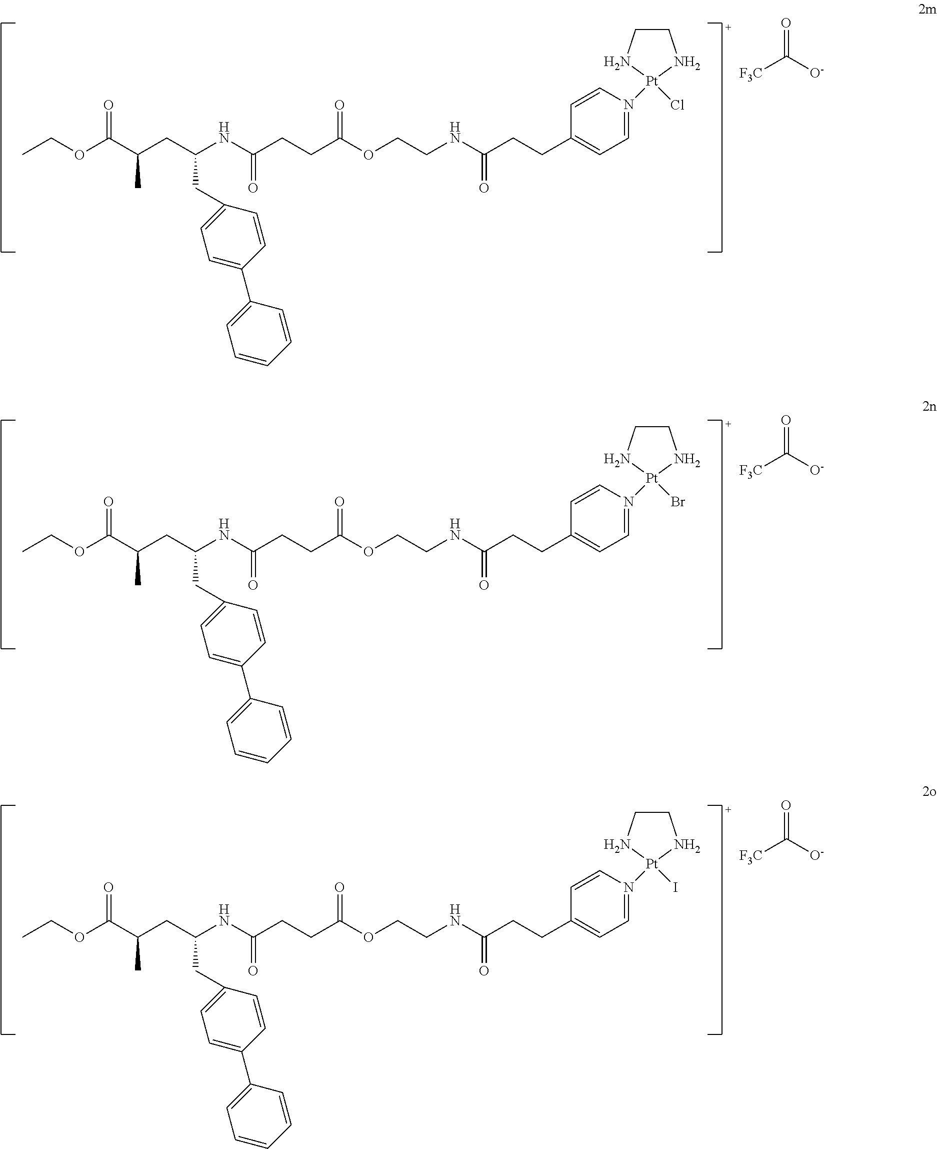

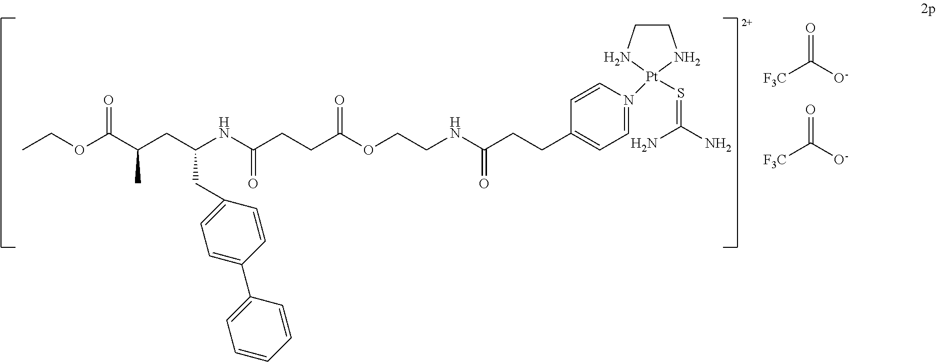

### 9.2.1. Synthesis and Analytical Characterization of Sacubitril-Py-Lx-I (2c)

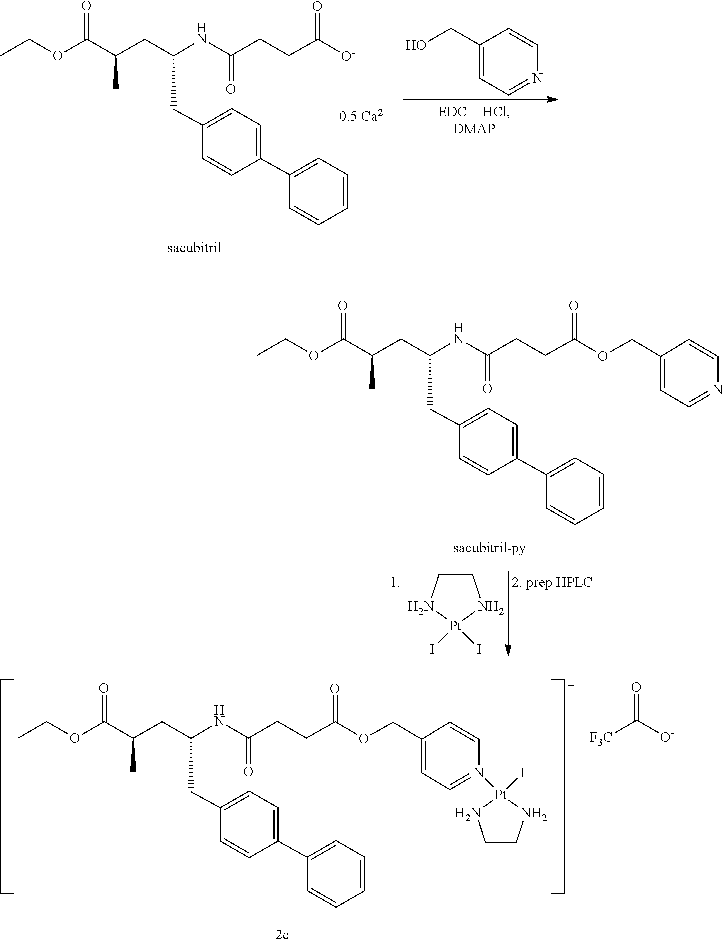

### 9.2.1.1. Synthesis and Analytical Characterization of Sacubitril-Py

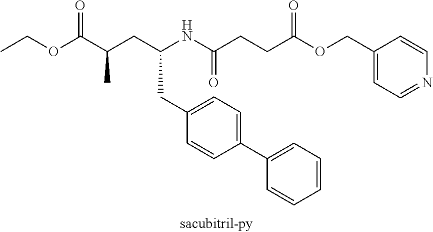 [SMILES: CCOC(=O)[C@H](C)C[C@@H](Cc1ccc(-c2ccccc2)cc1)NC(=O)CCC(=O)OCc1ccncc1]

Sacubitril hemicalcium (100 mg, 0.232 mmol, 1.0 eq.), pyridin-4-ylmethanol (30 mg, 0.279 mmol, 1.2 eq.), and DMAP (3 mg, 0.023 mmol, 0.1 eq.) were dissolved in dry DMF (1.5 mL). Subsequently, EDC×HCl (67 mg, 0.348 mmol, 1.5 eq.) and additional DMF (0.5 mL) were added. The resulting mixture was stirred at room temperature for four days under an argon atmosphere. The reaction mixture was diluted with water (20 mL) and extracted with EtOAc (2×20 mL). The organic phase was dried with Na₂SO₄, filtered, and the solvents were removed under reduced pressure. The residue was purified by column chromatography on silica (eluent: 0-3% MeOH/DCM) affording a colourless solid (73 mg, 62.4% yield).

¹H NMR (400 MHZ, CDCl₃): δ 8.58 (d, 2H), 7.60-7.54 (m, 2H), 7.54-7.49 (m, 2H), 7.46-7.40 (m, 2H), 7.37-7.30 (m, 1H), 7.27-7.22 (m, 4H), 5.62 (d, 1H), 5.13 (s, 2H), 4.31-4.19 (m, 1H), 4.13 (q, 2H), 2.89-2.80 (m, 2H), 2.76-2.69 (m, 2H), 2.66-2.50 (m, 2H), 2.49-2.42 (m, 2H), 1.99-1.90 (m, 1H), 1.59-1.49 (m, 1H), 1.24 (t, 3H), 1.16 (d, 3H).

HRMS (ESI⁺) C₃₀H₃₅N₂O₅ [M+H]⁺ calc 503.2540, found 503.2599.

### 9.2.1.2. Synthesis and Analytical Characterization of Sacubitril-Py-Lx-I (2c)

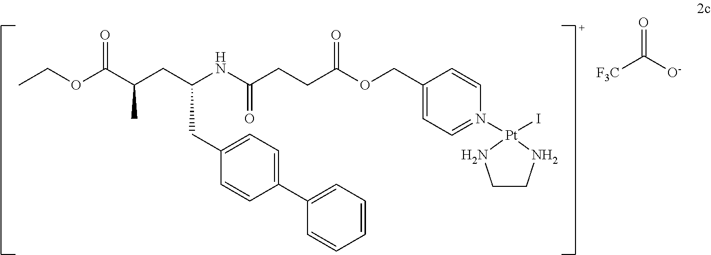

Pt (ethane-1,2-diamine)I₂ (LxI₂; 76.0 mg, 149.2 μmol, 5.0 eq.) was dissolved in a solution of sacubitril-py (15.0 mg, 29.8 μmol, 1.0 eq.) in dry DMF (200 μL) and the reaction mixture was shaken for 22 h at 25° C. The reaction mixture was then diluted with water/MeOH (1:1, 3 mL) and filtered through a 0.2 μm syringe filter. Purification was performed by preparative reverse-phase HPLC (Grace Alltima C18 5 μm column, 22× 250 mm; gradient: 45% to 100% B, whereas eluent A: 95/5 water/MeOH (+0.1% TFA) and eluent B: 5/95 water/MeOH (+0.1% TFA) in 40 min.). Product containing fractions were collected and lyophilized resulting in a yellow solid (14 mg, 47.0% yield).

HRMS (ESI⁺) C₃₂H₄₂IN₄O₅¹⁹⁵Pt [M]⁺ calc 884.1845, found 884.1856.

HPLC (Grace Alltima C18 5 μm column, 25×4.6 mm) indicated that the product was 95.8% pure (retention time 15.9 min.; gradient: 20% to 100% MeCN/0.1% TFA in water/0.1% TFA in 20 min. measured at a wavelength of 273 nm).

### 9.2.2. Synthesis and Analytical Characterization of Sacubitril-CH2CH2NHCO-Py-Lx-I (20)

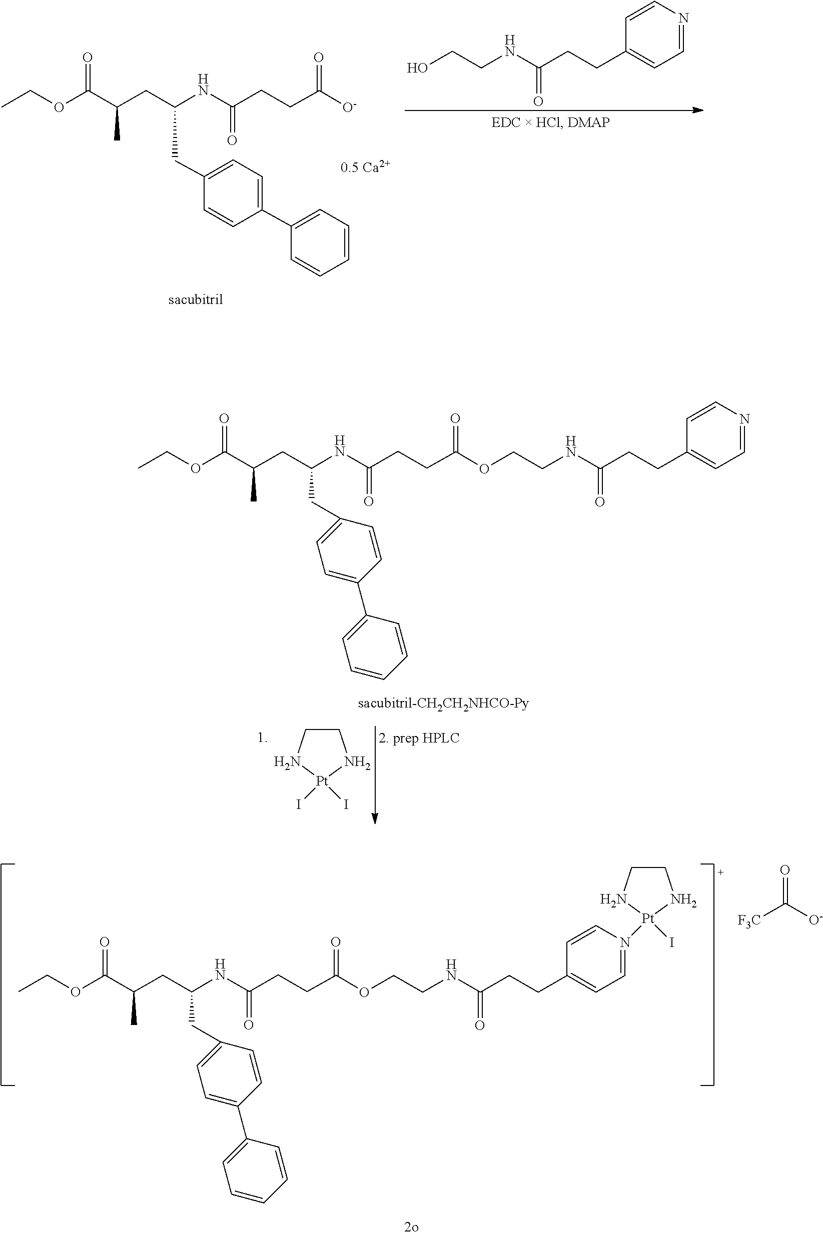

9.2.2.1. Synthesis and Analytical Characterization of 2,3,5,6-tetrafluorophenyl 3-(pyridin-4-yl) propanoate

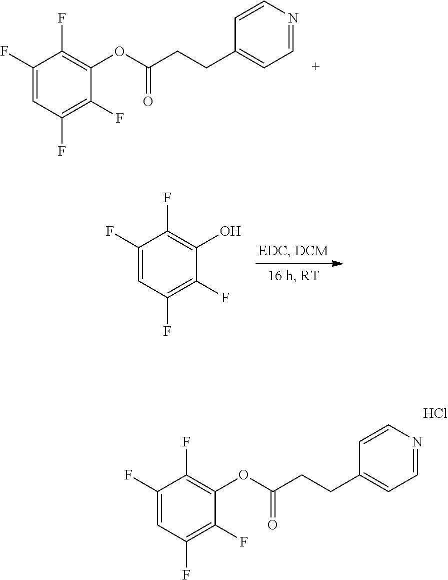 [SMILES: O=C(CCc1ccncc1)Oc1c(F)c(F)cc(F)c1F ; Oc1c(F)c(F)cc(F)c1F ; Cl ; O=C(CCc1ccncc1)Oc1c(F)c(F)cc(F)c1F]

3-(Pyridin-4-yl) propionic acid (500 mg, 3.3 mmol, 1.0 eq.), 2,3,5,6-tetrafluorophenol (604 mg, 3.6 mmol, 1.1 eq.) and EDC×HCl (761 mg, 4.0 mmol, 1.2 eq.) were dissolved in dry DCM (10 mL). The resulting mixture was stirred at room temperature for 16 h. Subsequently, the mixture was extracted with 0.1 M HCl (2×), brine (1×) and dried with Na₂SO₄ after which the solvent was removed under reduced pressure. The reaction afforded a white solid (530 mg, 47.7% yield).

¹H NMR (400 MHZ, CDCl₃): δ 8.49 (m, 2H), 7.91 (m, 1H), 7.35 (d, 2H), 3.21 (t, 2H), 3.02 (t, 2H).

9.2.2.2. Synthesis and Analytical Characterization of N-(2-hydroxyethyl)-3-(pyridin-4-yl) propanamide

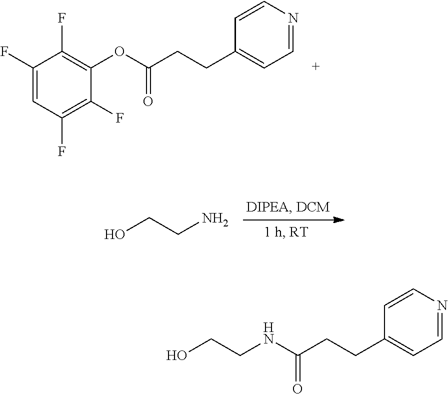

3-(Pyridin-4-yl) propionic-TFP (478 mg, 1.6 mmol, 1.0 eq.), 2-amino-ethanol (98 mg, 1.6 mmol, 1.0 eq.) and DIPEA (222 μL, 1.6 mmol, 1.0 eq.) were dissolved in dry DCM (10 mL). The resulting mixture was stirred at room temperature for 1 h. Subsequently, the solvent was removed under reduced pressure and the residue was purified by column chromatography on silica (eluent: 0-3% MeOH/DCM) affording a colourless solid (260 mg, 83.3% yield).

1H NMR (400 MHz, CD₃OD): δ 8.36-8.44 (m, 2H), 7.31 (d, 2H), 3.54 (t, 2H), 3.26 (t, 2H), 2.97 (t, 2H), 2.55 (t, 2H).

9.2.2.3. Synthesis and Analytical Characterization of sacubitril-CH2CH2NHCO-py

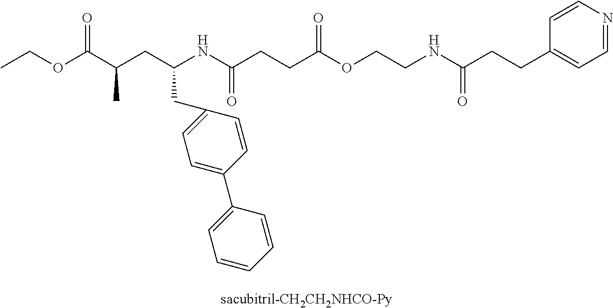 [SMILES: CCOC(=O)[C@H](C)C[C@@H](Cc1ccc(-c2ccccc2)cc1)NC(=O)CCC(=O)OCCNC(=O)CCc1ccncc1]

Sacubitril hemicalcium (200 mg, 0.487 mmol, 1.0 eq.), N-(2-hydroxyethyl)-3-(pyridin-4-yl) propanamide (114 mg, 0.584 mmol, 1.2 eq.), and DMAP (6 mg, 0.049 mmol, 0.1 eq.) were dissolved in dry DMF (1.5 mL). Subsequently, EDC×HCl (140 mg, 0.751 mmol, 1.5 eq.) and additional DMF (0.5 mL) were added. The resulting mixture was stirred at room temperature for 20 h under an argon atmosphere. The reaction mixture was diluted with water (20 mL) and extracted with EtOAc (2×20 mL). The organic phase was dried with Na₂SO₄, filtered, and the solvents were removed under reduced pressure. The residue was purified by column chromatography on silica (eluent: 0-5% MeOH/DCM) affording a colourless solid (99 mg, 33.5% yield).

HRMS (ESI⁺) C₃₅H₄₁IN₃O₆ [M+H]⁺ calc 588.3068, found 588.3083.

HPLC (Grace Alltima C18 5 μm column, 25×4.6 mm) indicated that the product was 100% pure (retention time 14.0 min.; gradient: 20% to 100% MeCN/0.1% TFA in water/0.1% TFA in 20 min. measured at a wavelength of 273 nm).

9.2.2.4. Synthesis and Analytical Characterization of sacubitril-CH2CH2NHCO-py-Lx-I (20)

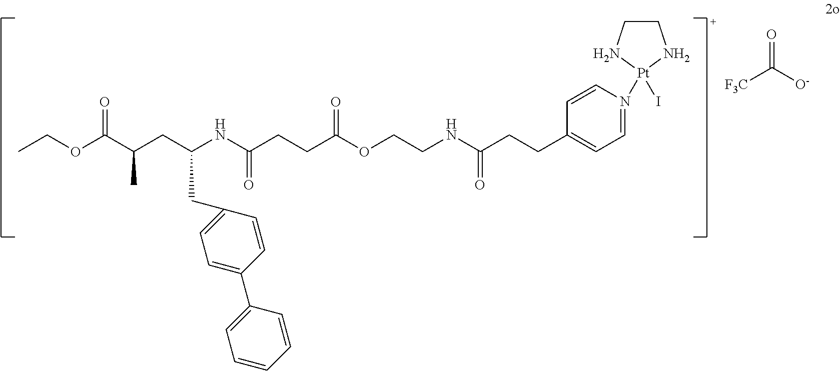

Pt (ethane-1,2-diamine)I₂ (LxI₂; 34.6 mg, 272.8 μmol, 4.0 eq.) was dissolved in a solution of sacubitril CH₂CH₂NHCO-py (40.0 mg, 68.2 μmol, 1.0 eq.) in dry DMF (416 μL) and the reaction mixture was shaken for 27 h at 50° C. The reaction mixture was then diluted with water/MeOH (1:1, 3 mL) and filtered through a 0.2 μm syringe filter. Purification was performed by preparative reverse-phase HPLC (Grace Alltima C18 5 μm column, 22×250 mm; gradient: 65% to 90% B, whereas eluent A: 95/5 water/MeOH (+0.1% TFA) and eluent B: 5/95 water/MeOH (+0.1% TFA) in 40 min.). Product containing fractions were collected and lyophilized resulting in a yellow solid (26.7 mg, 36.2% yield).

HRMS (ESI⁺) C₃₆H₄₉IN₅O₆¹⁹⁵Pt [M]⁺ calc 969.2370, found 969.2388.

HPLC (Grace Alltima C18 5 μm column, 25×4.6 mm) indicated that the product was 96.4% pure (retention time 15.2 min.; gradient: 20% to 100% MeCN/0.1% TFA in water/0.1% TFA in 20 min. measured at a wavelength of 273 nm).

9.3. Structures of Pacritinib-Lx SFMs and Maleimide Functionalized Pacritinib Moieties that can be Utilized in the Context of the Present Invention

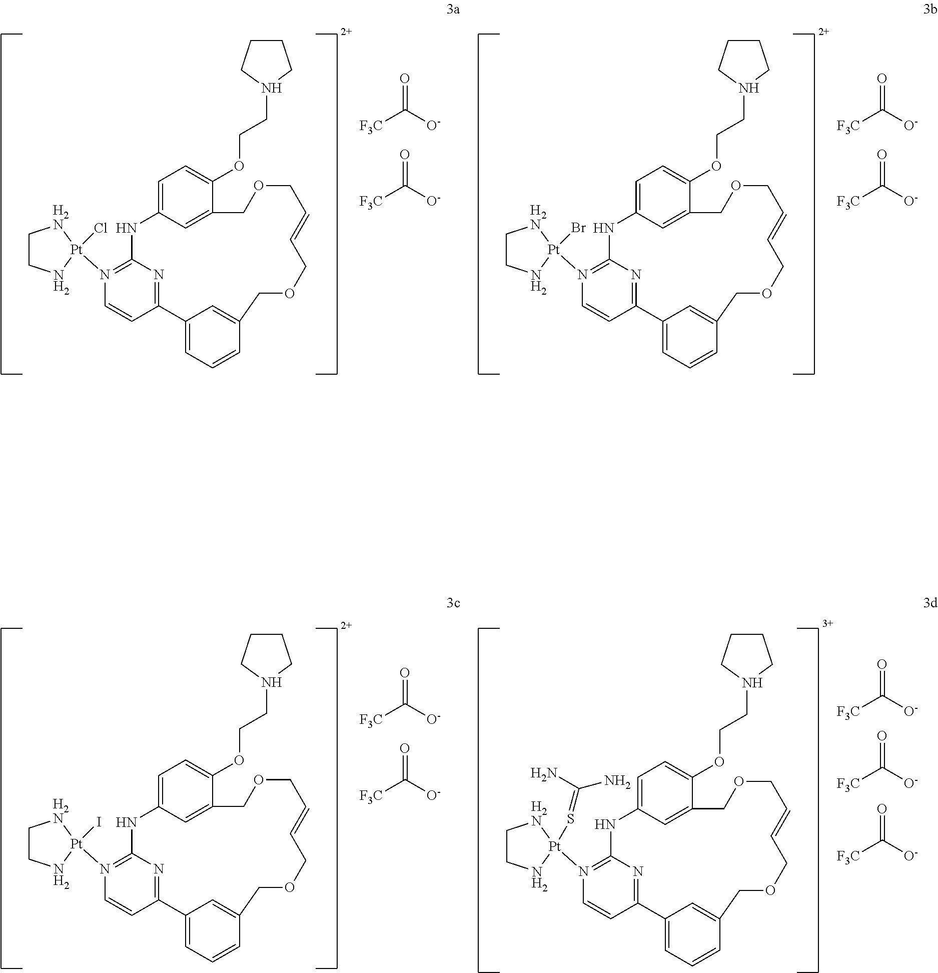

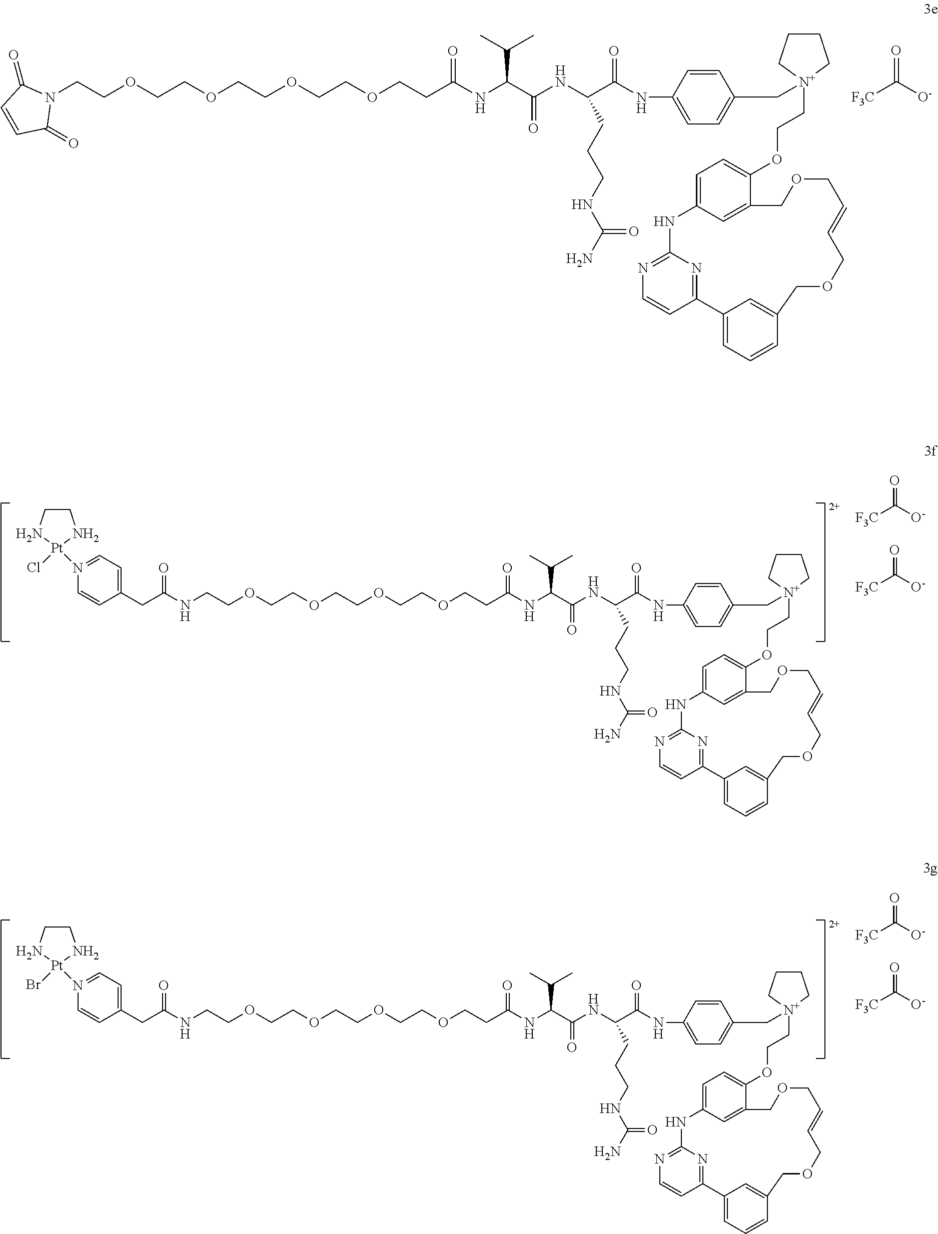

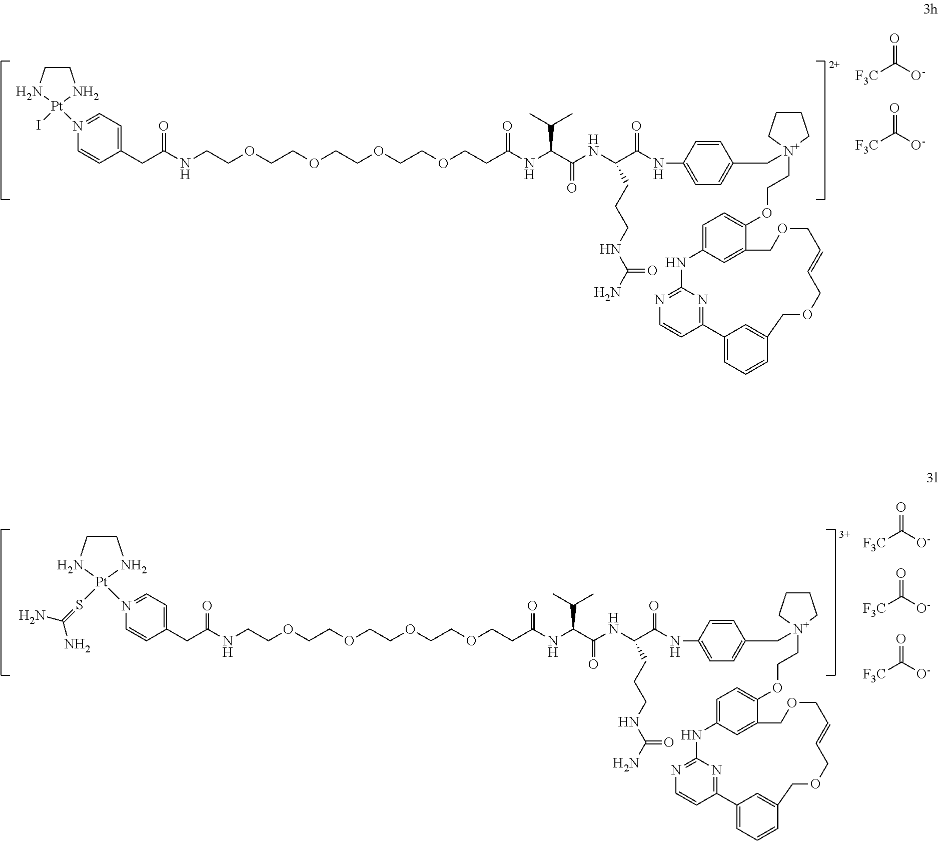

### 9.3.1. Synthesis and Analytical Characterization of Pacritinib-Lx-I (3c)

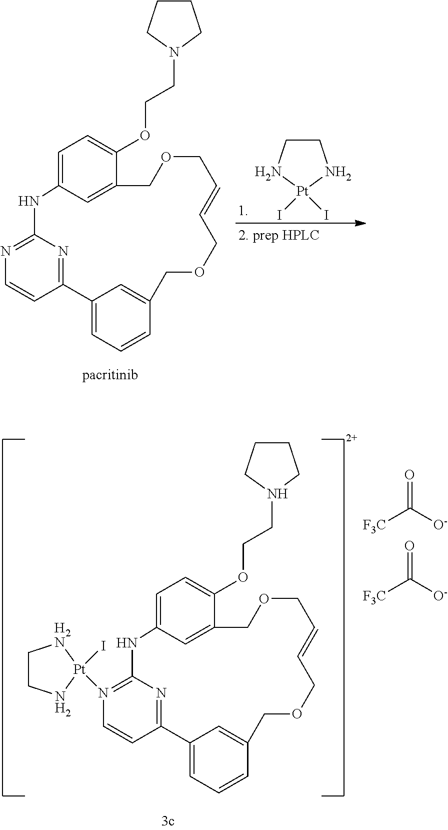

Pacritinib (20 mg, 42.3 μmol, 1 eq.) and Pt (ethane-1,2-diamine)I₂ (LxI₂; 96.9 mg, 190.4 μmol, 4.5 eq.) were dissolved in dry DMF (250 μL) and the reaction mixture was shaken for 48 h at 60° C. The reaction mixture was then diluted with water/MeOH (1:1, 3 mL) and filtered through a 0.2 μm syringe filter. Purification was performed by preparative reverse-phase HPLC (Grace Alltima C18 5 μm column, 22× 250 mm; gradient: 45% to 80% B, whereas eluent A: 95/5 water/MeOH (+0.1% TFA) and eluent B: 5/95 water/MeOH (+0.1% TFA) in 40 min.). Product containing fractions were collected and lyophilized and the product 3c was obtained as a yellow solid (13.1 mg, 28.6% yield).

HRMS (ESI⁺) C₃₀H₄₁IN₆O₃¹⁹⁵ Pt [M]²⁺ calc 427.5962, found 427.5999.

HPLC (Grace Alltima C18 5 μm column, 25×4.6 mm) indicated that the product was 92.7% pure (retention time 10.7 min.; gradient: 20% to 100% MeCN/0.1% TFA in water/0.1% TFA in 20 min. measured at a wavelength of 273 nm).

### 9.3.2. Synthesis and Analytical Characterization of Mal-PEG4-Val-Cit-PAB-pacritinib (3e)

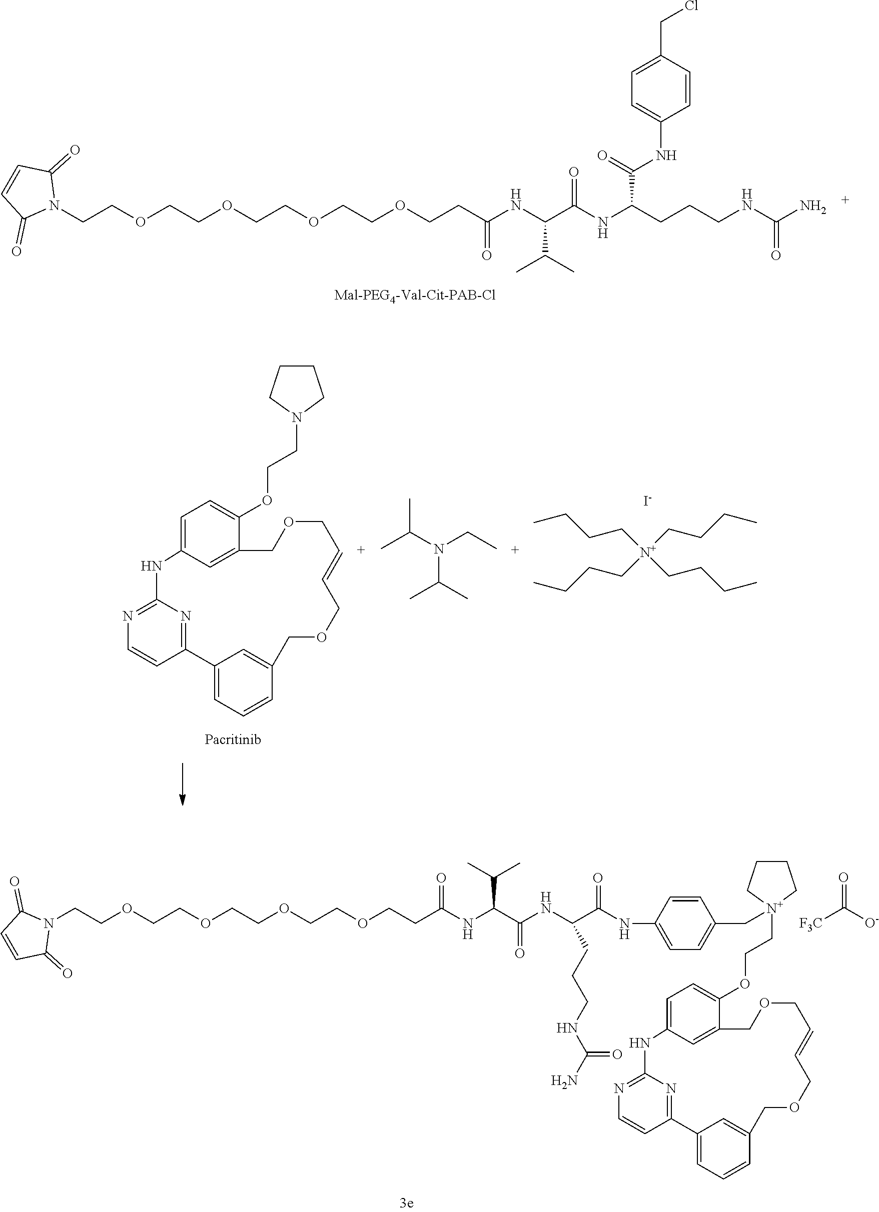

### 9.3.2.1. Synthesis and Analytical Characterization of Mal-PEG4-Val-Cit-PAB-CI

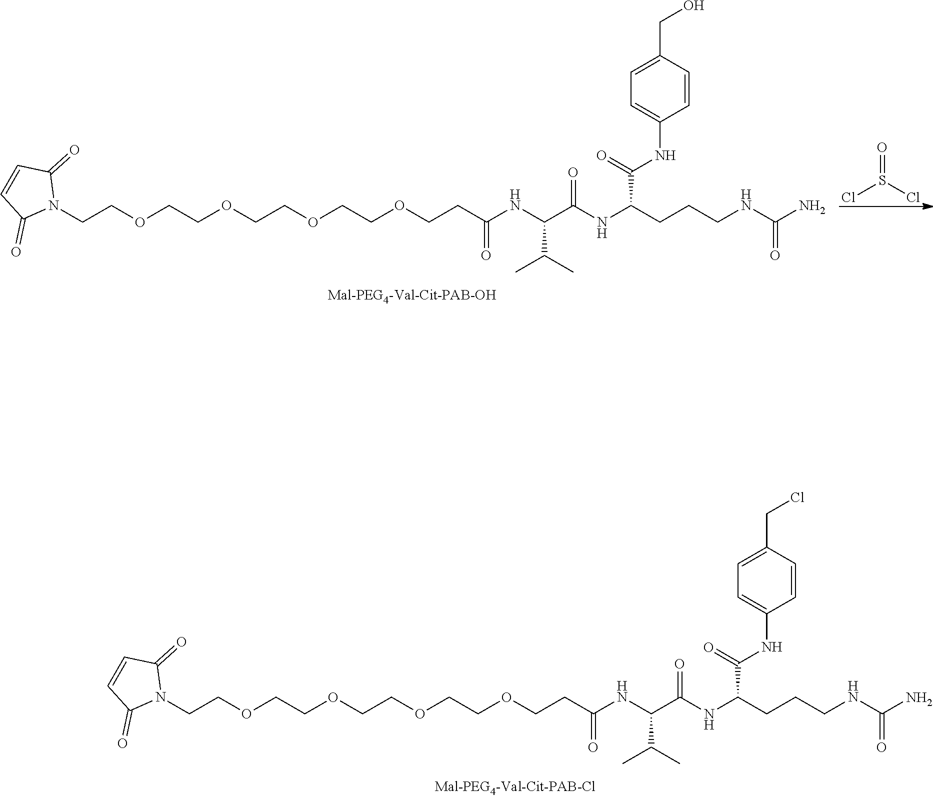

Mal-PEG₄-Val-Cit-OH (10 mg, 14.2 μmol, 1.0 eq.) was dissolved in DMF (200 μL) and cooled to 0° C. in an ice/water bath. Subsequently, thionyl chloride (1.9 mg, 15.6 μmol, 1.1 eq.) was added and the mixture was stirred for 15 min. at 0° C. after which the solvent was removed under reduced pressure.

HRMS (ESI⁺) C₃₃H₄₉³⁵ClN₆O₁₀ [M+H]⁺ calc 725.3271, found 725.3298.

HPLC (Grace Alltima C18 5 μm column, 25×4.6 mm) indicated that the product was >90% pure (retention time 9.6 min.; gradient: 20% to 50% B, whereas eluent A: 95/5 water/MeCN (+0.1% TFA) and eluent B: 5/95 water/MeCN (+0.1% TFA) in 20 min. measured at a wavelength of 273 nm).

### 9.3.2.2. Synthesis and Analytical Characterization of Mal-PEG4-Val-Cit-PAB-pacritinib (3e)

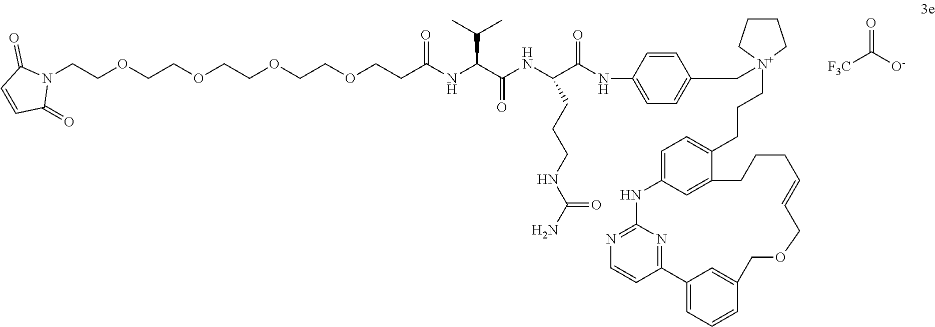 [SMILES: CC(C)[C@H](NC(=O)CCOCCOCCOCCOCCN1C(=O)C=CC1=O)C(=O)N[C@@H](CCCNC(N)=O)C(=O)Nc1ccc(C[N+]2(CCCc3ccc4cc3CCC/C=C/COCc3cccc(c3)-c3ccnc(n3)N4)CCCC2)cc1 ; O=C([O-])C(F)(F)F]

Pacritinib (9.9 mg, 21.0 μmol, 1.0 eq.) was dissolved in DMF (250 μL) and added to Mal-PEG4-Val-Cit-CI (15.2 mg, 21.0 μmol, 1.0 eq.) dissolved in DMF (312.5 μL). Subsequently, DIPEA (6.8 mg, 52.4 μmol, 2.5 eq.) and tetrabutylammonium iodide were added (1.6 mg, 4.2 μmol, 0.2 eq.) and the mixture was stirred for 21 h at 30° C.

The reaction mixture was then diluted with dry DMSO (1.5 mL) and filtered through a 0.2 μm syringe filter. Purification was performed by preparative reverse-phase HPLC (Grace Alltima C18 5 μm column, 22×250 mm; gradient: 20% to 50% B, whereas eluent A: 95/5 water/MeCN (+0.1% TFA) and eluent B: 5/95 water/MeCN (+0.1% TFA) in 40 min.). Product containing fractions were collected and lyophilized and the product 3e was obtained as a yellow solid (15.3 mg, 57.3% yield).

HRMS (ESI⁺) C₆₁H₈₁N₁₀O₁₃ [M+H]⁺ calc 1161.5979, found 1161.5873.

HPLC (Grace Alltima C18 5 μm column, 25×4.6 mm) indicated that the product was 96.3% pure (retention time 12.1 min.; gradient: 20% to 100%, whereas eluent A: 95/5 water/MeCN (+0.1% TFA) and eluent B: 5/95 water/MeCN (+0.1% TFA) in 20 min, measured at a wavelength of 273 nm).

9.4. Structures of Losartan-Lx SFMs that can be Utilized in the Context of the Present Invention

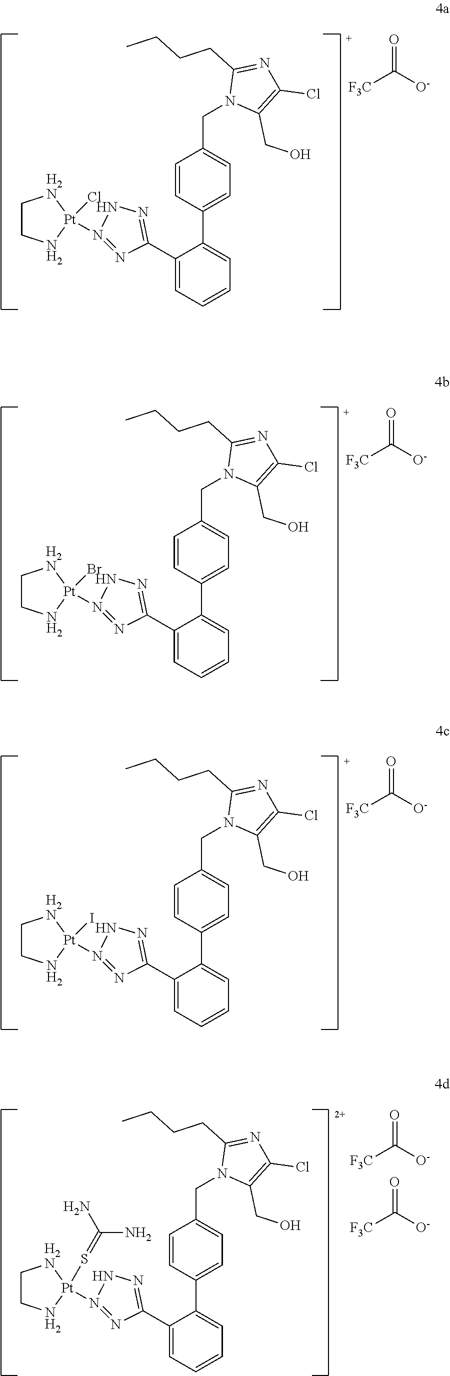

### 9.4.1. Synthesis and Analytical Characterization of Losartan-Lx-Cl (4a)

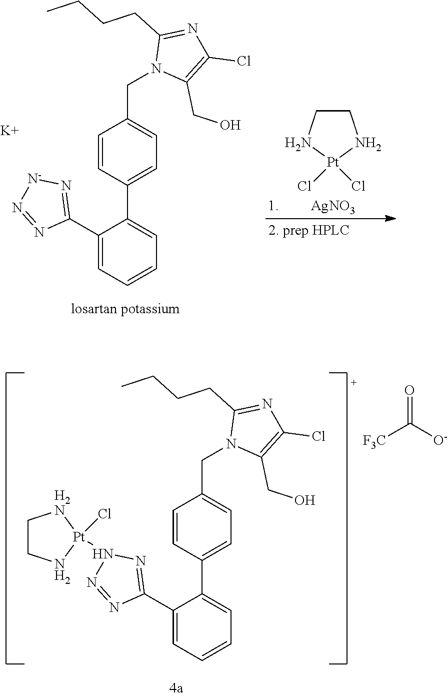

AgNO₃ (26.1 mg, 153.3 μmol, 1.0 eq.) was added to a suspension of PtCl₂ (ethane-1,2-diamine) (LxCl₂; 50 mg, 153.3 μmol, 1.0 eq.) in dry DMF (8.3 mL) and stirred overnight at room temperature in the dark under argon atmosphere. The suspension was then filtered through a 0.2 μm syringe filter to give a 18.5 mM stock solution of the activated Pt-complex. Then, to a solution of losartan potassium (20 mg, 43.4 μmol, 1.0 eq.) in dry DMF (500 μL), the above prepared 18.5 mM stock solution of the activated Pt-complex (1.76 mL, 32.5 μmol, 0.75 eq.) was added. The reaction mixture was stirred for 1.5 h at 22° C. Subsequently, 1 M HCl (70.4 μL, 2 eq.) and 0.9% NaCl (1 mL) were added to the mixture, after which the solvents were removed under reduced pressure. The residue was dissolved in MilliQ water/MeOH (1:1, 3 mL) and filtered through a 0.2 μm syringe filter. Purification was performed by preparative reverse-phase HPLC (Grace Alltima C18 5 μm column, 22×250 mm; gradient: 20% to 50% B, whereas eluent A: 95/5 water/MeCN (+0.1% TFA) and eluent B: 5/95 water/MeCN (+0.1% TFA) in 40 min.). Product containing fractions were lyophilized and the product 4a was obtained as a colourless solid (11 mg, 47.4% yield).

HRMS (ESI⁺) C₂₄H₃₁³⁵Cl₂N₈O¹⁹⁵Pt [M]⁺ calc 712.1641, found 712.1628.

HPLC (Grace Alltima C18 5 μm column, 25×4.6 mm) indicated that the product was 94.3% pure (retention time 9.6 min.; gradient: 5% to 25% MeCN/0.1% TFA in water/0.1% TFA in 18 min. measured at a wavelength of 273 nm).

### Example 10: The Conjugation of a Semi-Final Complex and of a Maleimide Functionalized Moiety to a Binding Molecule of the Invention

### 10.1. Conjugation of Lx SFMs to a Binding Molecule

Biparatopic 13F11-13E8 (MW˜27.9 kD, 105 μL, 5 nmol, 1.33 mg/mL, 1.0 eq.) was diluted with borate buffer (20 μL, 250 mM sodium borate, 250 mM NaCl, and 10 mM diethylenetriaminepentaacetic acid, pH 8.0) and H₂O (200 μL), after which a solution of tris(2-carboxyethyl) phosphine hydrochloride (TCEP×HCl; 2 μL, 10 mM in H₂O, 10 nmol, 2.0 eq.) was added. The mixture was incubated in a thermoshaker at 37° C. for 2 h. Simultaneously, a solution of the SFM 1c (10 μL, 5 mM in 20 mM NaI, 50 nmol, 10.0 eq.) was mixed with an aqueous solution of thiourea (10 μL, 20 mM) and incubated in a thermoshaker at 37° C. for 2 h. Subsequently, the above prepared solutions of a binding molecule of the invention and the thiourea treated SMF were mixed and incubated in a thermoshaker at 37° C. for 1 h. The conjugates were purified by spin filtration using 10 kDa MWCO filters (washed 4× with PBS), after which they were reconstituted and stored in PBS. The Y27632-to-13F11-13E8 ratio as determined by SEC-MS was 1.0.

### 10.2. Conjugation of a Maleimide Functionalized Moieties to a Binding Molecule

Biparatopic 13F11-13E8 (MW˜27.9 kD, 105 μL, 5 nmol, 1.33 mg/mL, 1.0 eq.) was diluted with borate buffer (20 μL, 250 mM sodium borate, 250 mM NaCl, and 10 mM diethylenetriaminepentaacetic acid, pH 8.0) and H₂O (200 μL), after which a solution of tris(2-carboxyethyl) phosphine hydrochloride (TCEP×HCl; 2 μL, 10 mM in H₂O, 120 nmol, 2.0 eq.) was added. The mixture was incubated in a thermoshaker at 37° C. for 2 h. Subsequently, the above prepared solution of a binding molecule of the invention was mixed with the maleimide functionalized moiety 1e (2.5 μL, 10 mM in DMSO, 50 nmol, 10.0 eq.) and incubated at 0° C. for 1 h. The conjugates were purified by spin filtration using 10 kDa MWCO filters (washed 4× with PBS), after which they were reconstituted and stored in PBS. The Y27632-to-13F11-13E8 ratio as determined by SEC-MS was 1.0.

### Example 11: Internalization of VHHs

A biparatopic design should confer superior internalization ability to VHH constructs when compared to its monomeric VHH elements. Superior internalization is key to the present invention.

### 11.1. IGF2R VHH

Internalizing ability of IGF2R-binding VHH constructs and (their) corresponding monomeric VHHs was assessed in various approaches.

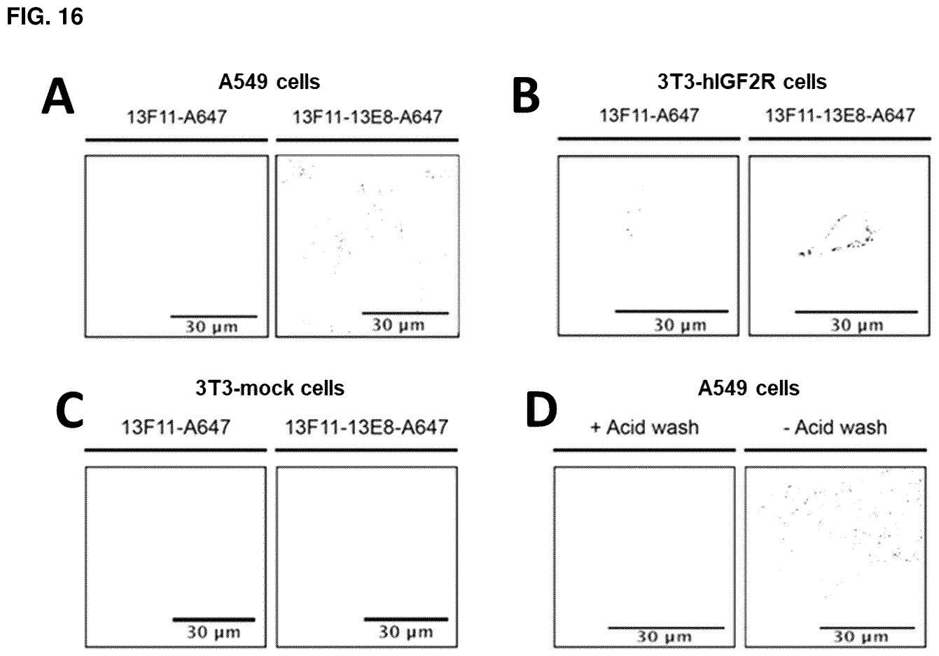

First, Alexa fluor 647-conjugates of the VHH 13F11 and the biparatopic VHH construct 3F11-13E8 were added to A549 cells that endogenously express hIGF2R (FIG. 16A), NIH 3T3 2.2 cells that were transiently transfected with hIGF2R (FIG. 16B), or mock transfected NIH 3T3 2.2 cells (as a negative control). For this, the cells were incubated with 25 nM of the VHH or the VHH construct. Before fluorescent microscopy readout, to ensure that only signal of internalized VHH or mouse anti-IGF2R was present, an acid wash (0.2M glycine-HCl, 150 mM NaCl, pH 2.3) was used to remove VHH or mouse anti-IGF2R bound to the target receptors on the cell surface. The efficiency of the acid wash procedure was verified by incubating the hIGF2R transfected NIH 3T3 2.2 cells with 13F11-13E8-A647 on ice and subsequently treating these cells with an acid wash while remaining on ice to prevent internalization (FIG. 16D). In A549 cells, 13F11-13E8 clearly internalized whereas for monovalent 13F11 no internalization was observed (FIG. 16A). In NIH 3T3 2.2 cells transiently transfected with hGF2R, 13F11 as well as 13F11-13E8 internalized but the biparatopic construct showed superior internalization (FIG. 16B). As expected, no internalization was observed in mock transfected NIH 3T3 2.2 cells (FIG. 16C). The acid wash control experiment (using 13F11-13E8) demonstrated that the acid wash step was fully effective in removing membrane-bound VHH, validating the assay to be selective for internalization (FIG. 16D). Summarizing, biparatopic 13F11-13E8 clearly internalizes in cells expressing hIGF2R on the cell surface.

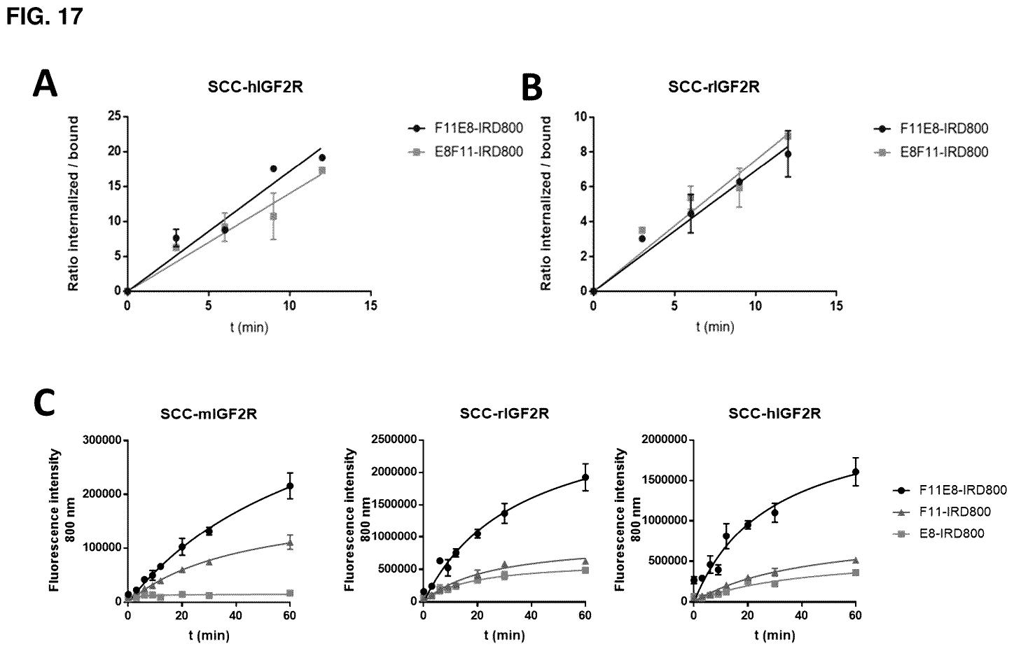

The rate of internalization (endocytic rate constant, kₑ) of biparatopic constructs 13F11-13E8 and 13E8-13F11 was determined with a kinetic internalization assay on SCC-hIGF2R and SCC-rIGF2R cells. Briefly, 10 nM of IRD800CW conjugated 13F11-13E8 or 13E8-13F11 (conjugation method as described in example 10.2) was added to hIGF2R expressing SCC cells or rIGF2R expressing SCC cells and incubated at 37° C. for different time points up to 12 minutes. Internalization was then purposely halted by putting the cells on ice. Subsequently, cells were washed with cold PBS and the non-internalized VHH construct (bound fraction) was separated from the internalized construct (internalized fraction) using an acid-wash, as described above, after which the fluorescence of both fractions were measured. Both constructs internalize with similar endocytic rate constants in SCC-hIGF2R cells (kₑ around 1.5 min-1) and in SCC-rIGF2R cells (kₑ around 0.7 min-1) (FIGS. 17A and B, respectively).

Internalization of 13F11-13E8 and the corresponding monomeric VHHs 13F11 and 13E8 was assessed in a similar assay (FIG. 17A-C). As shown in FIG. 17, as compared to the monomeric VHHs, the biparatopic construct showed superior internalization in SCC cells expressing mIGF2R, rIGF2R and hIGF2R (FIG. 17 C, left, middle and right panel, respectively).

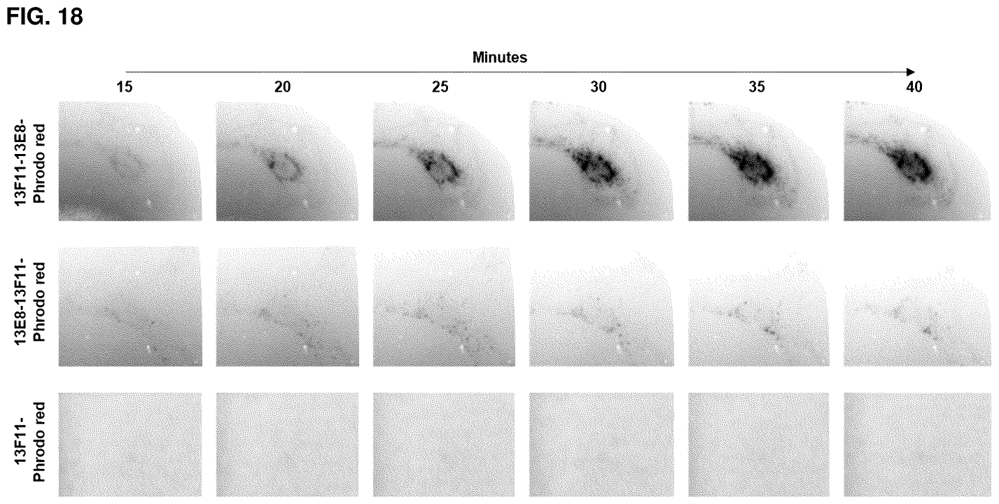

Internalization of monovalent 13F11 and biparatopics 13F11-13E8 and 13E8-13F11 was finally assessed in LX2 cells (an activated hepatic stellate cell line). For live imaging of internalization, the pH dependent Phrodo probe was used (ThermoFisher Scientific). This pH dependent probe becomes more fluorescent at acidic pH. Endosomes and lysosomes have a pH maintained at 6.5 and 4.5, respectively, compared to cytoplasmic pH of 7.0, and thus internalization of a VHH-pHrodo construct can be visualized when it internalizes via the endosomal pathway. LX-2 cells were incubated at 37° C. for 2 hours with 50 nM of pHrodo-red conjugated 13F11, 13F11-13E8 and 13E8-13F11 (conjugation method as described in example 10.2). Images were taken at 5 min. intervals using a wide-field PEXScope microscope (Nikon) with a mCherry filter and 40× oil objective. Compared to the monovalent construct, a more pronounced increase in intracellular fluorescence was observed from 15 min. onwards for both biparatopic constructs 13F11-13E8 and 13E8-13F11 (FIG. 18). Biparatopic 13F11-13E8 clearly shows higher increase of fluorescence over the 40-min. time period compared to 13E8-13F11 indicating a higher rate of internalization. Additionally, both internalized 13F11-13E8 and 13E8-13F11 accumulate into perinuclear compartments (endosomes/lysosomes).

### 11.2. PDGFRB VHH

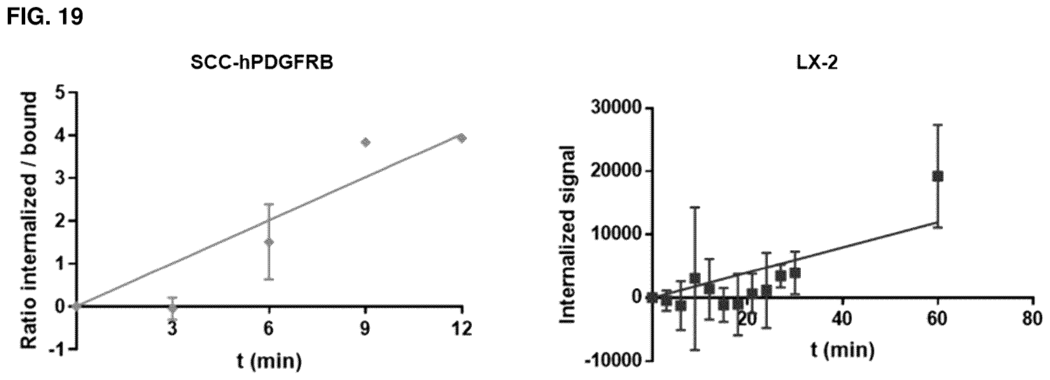

To determine the rate of internalization of the VHH construct SP02P-SP26P, kinetic internalization assays on hPDGFRB expressing SCC cells and LX-2 cells were performed using a similar method as described for the IGF2R constructs in example 11.1. Internalization of SP02P-SP26P was observed in SCC-hPDGFRB cells (left panel of FIG. 19) as well as in LX-2 cells (right panel of FIG. 19).

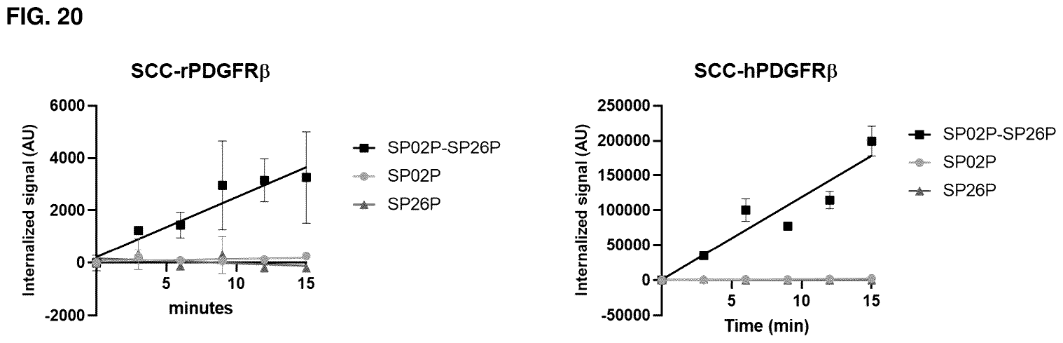

To confirm improved internalization of biparatopic VHH constructs compared to the monomeric individual VHH components, kinetic internalization assays were performed using SCC-rPGDFRB and on SCC-hPDGFRB cells, using a similar method as described for the IGF2R constructs in example 11.1. The biparatopic construct SP02P-SP26P internalized 5-10 fold faster when compared to the corresponding monomeric VHH SP02 and SP26 (FIG. 20; left panel for cells expressing rat PDGFRB, right panel for cells expressing human PDGFRB).

### Example 12: NDC-Lx-Pacritinib Induces Relaxation of Activated Hepatic Stellate Cells (HSCs) in a Contraction Assay

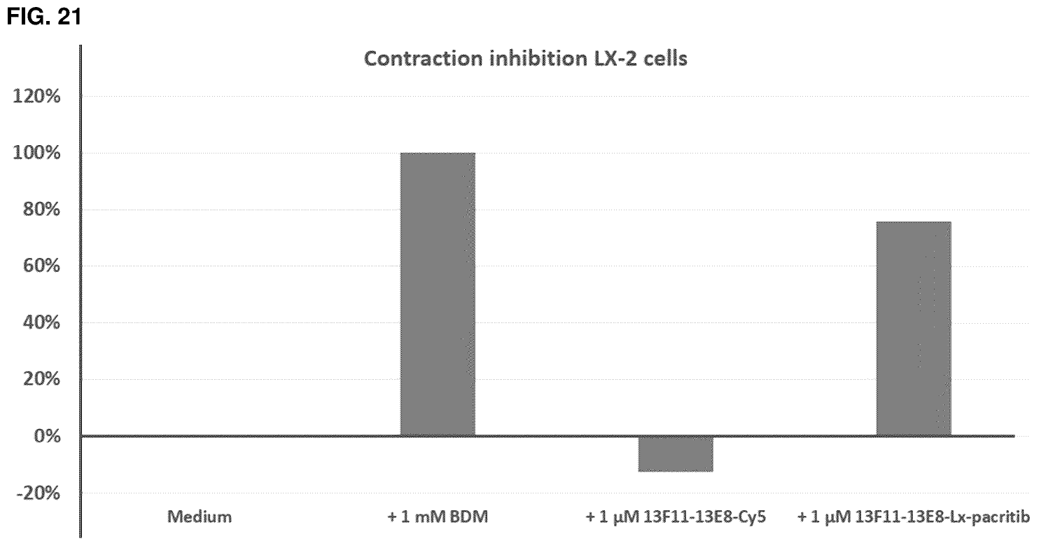

The relaxing potency of biparatopic VHH 13F11-13E8 conjugated to the JAK2 inhibitor pacritinib (3c) (conjugation method as described in example 10.1.) was assessed in vitro using a contraction assay with the human HSC line LX-2. Wells of a 24-wells plate were filled with 0.5 mL BSA and left for at 37° C. for 1 h after which the wells were washed with PBS and dried. Subsequently, a mixture of 1M NaOH, 10×PBS, sterile water, 2×DMEM, 0.2M HEPES and 1 mg/mL type I collagen was added to the wells and incubated at 37° C. for 1 h allowing gelation of the collagen. The LX-2 cells, which medium was replaced with DMEM supplemented with 2% FBS 24 h prior to the assay, were harvested and resuspended at 2×10⁵ cells per mL medium (2% FCS in DMEM) and 500 μl was added to the gel and incubated at 37° C. for 3 h. After attachment of the cells to the collagen gel, the medium was removed and replaced with 500 μl of fresh medium with 1 μM 13F11-13E8-Lx-pacritinib, 1 μM 13F11-13E8-Cy5 (conjugation method as described in 10.2., negative control) or 1 μM contraction inhibitor 2,3-butanedione monoxime (BDM, positive control). The collagen gels were detached from the sides of the wells by moving a pipette tip or spatula around the edges and the cells were incubated at 37° C. up to 7 days. Every 24 h, pictures of the collagen gels were taken with a camera (JAI CV-A55-IR) at a fixed distance or height. The areas of the collagen gels were quantified in pixels using Adobe Photoshop (CC 2017). The percentage of contraction inhibition was determined setting the gel incubated without any inhibitor at 0% contraction inhibition and the gel incubated at 1 μM BDM (positive control) at 100% contraction inhibition. Construct 13F11-13E8-Lx-pacritinib was able to induce contraction inhibition up to 76%, whereas a nanobody construct not equipped with a kinase inhibitor (13F11-13E8-mal-Cy5) did not inhibit contraction (FIG. 21).

### Example 13: Detection of IGF2R in the Cirrhotic Rat Liver

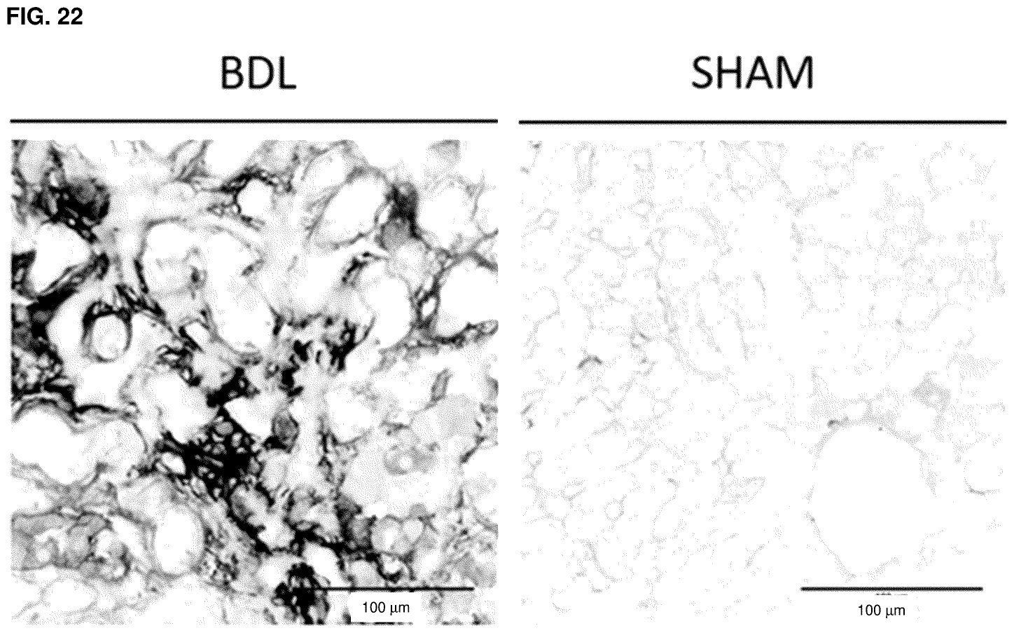

To evaluate whether F11E8-ABD bound to IGF2R as (over-) expressed in the cirrhotic liver, various cryo-sections of tissues (liver, kidney, spleen, heart, ileum, brain) obtained from both healthy (SHAM) and cirrhotic (BDL) rats (preparation procedure as outlined in example 15) were stained with 13F11-13E8-ABD conjugated to Alexa Fluor 647 (13F11-13E8-ABD-A647, conjugation method as described in example 10.2.). Staining was performed with 50 nM 13F11-13E8-ABD-AF647 during overnight incubation at 4° C. Subsequently, the sections were washed and stained with DAPI, mounted with mowiol mounting medium (Merck) and imaged on a Zeiss LSM700 laser scanning confocal microscope with a 20× objective. 13F11-13E8-ABD-AF647 strongly stains activated fibroblasts in the cirrhotic liver whereas in the normal liver only background signal was detected (FIG. 22). In the tissues other than the liver, no upregulation of IGF2R was observed. These observations demonstrate binding of the VHH construct to rIGF2R in its biological context, and confirm selective over-expression of IGF2R by activated fibroblasts, in line with what is reported elsewhere (see example 1).

### Example 14: Detection of PDGFRB in the Cirrhotic Rat Liver

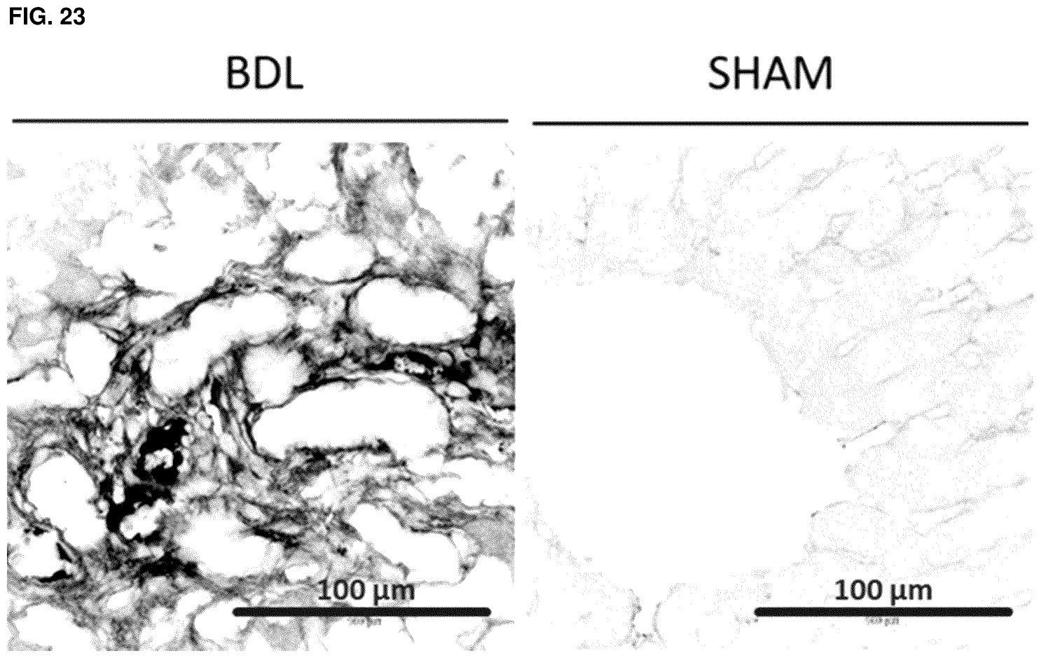

To evaluate whether SP02-SP26-ABD bound to PDGFRB as (over-) expressed in the cirrhotic liver, various cryo-sections of tissues (liver, kidney, spleen, heart, ileum, brain) obtained from both healthy (SHAM) and cirrhotic (BDL) rats (preparation procedure as outlined in example 15) were stained with SP02-SP26-ABD conjugated to Alexa Fluor 647 (SP02-SP26-ABD-A647, conjugation method as described in example 10.2.). Staining was performed with 50 nM SP02-SP26-ABD-AF647 during overnight incubation at 4° C. Subsequently, the sections were washed and stained with DAPI, mounted with mowiol mounting medium (Merck), and imaged on a Zeiss LSM700 laser scanning confocal microscope with a 20× objective. SP02-SP26-ABD-AF647 strongly stains activated fibroblasts in the cirrhotic liver whereas in the normal liver only background signal was detected (FIG. 23). In the tissues other than the liver, no upregulation of PDGFRB was observed. These observations demonstrate binding of the VHH construct to rPDGFRB in its biological context, and confirm selective over-expression of PDGFRB by activated fibroblasts, in line with what is reported elsewhere (see example 1).

### Example 15: Selective Lowering of Portal Hypertension by Targeting Activated Hepatic Stellate Cells In Vivo with an IGF2R-Binding Molecule of the Invention Conjugated to a Small Molecule Kinase Inhibitor

To evaluate in vivo functionality, 13F11-13E8-ABD was conjugated to a Y27632-maleimide modified functional moiety (1e) (the test conjugate) as outlined in example 10.2. To provide a comparator, a similar amount of 13F11-13E8-ABD was conjugated to N-ethylmaleimide in a parallel and similar preparation (the mock conjugate).

To establish animals displaying portal hypertension (Klein et al, Fibrosis: Methods and Protocols. Methods in Molecular Biology. 2017 (1627): 91-122.), Sprague-Dawley rats were subjected to bile duct ligation (BDL) through median laparotomy under ketamine/xylazine-induced anaesthesia i.p. (BDL rats). To provide healthy controls, a second cohort of rats was subjected to the same surgical procedure except for actual ligation of the bile duct (SHAM rats). Surgery was followed-up with pain control medication (carprofen s.c.). Approximately five weeks after surgery, ketamine/xylazine anesthetized rats were subjected to median laparotomy, after which one catheter was inserted through an ileocecal vein to the portal vein, and one catheter was inserted into the femoral artery. The catheters were connected to pressure recording equipment, for assessment of portal pressure and mean arterial pressure, respectively.

After catheterization and hemodynamic stabilization, rats were dosed with 5 mg/kg test conjugate or mock conjugate i.v. through the lateral tail vein and pressures were monitored continuously (example 15.1.). One hour after dosing, animals were sacrificed after which tissues were isolated for ex vivo analysis (example 15.2.).

### 15.1. Selective Lowering of Portal Pressure by an IGF2R-Binding Molecule of the Invention Conjugated to a Small Molecule Kinase Inhibitor

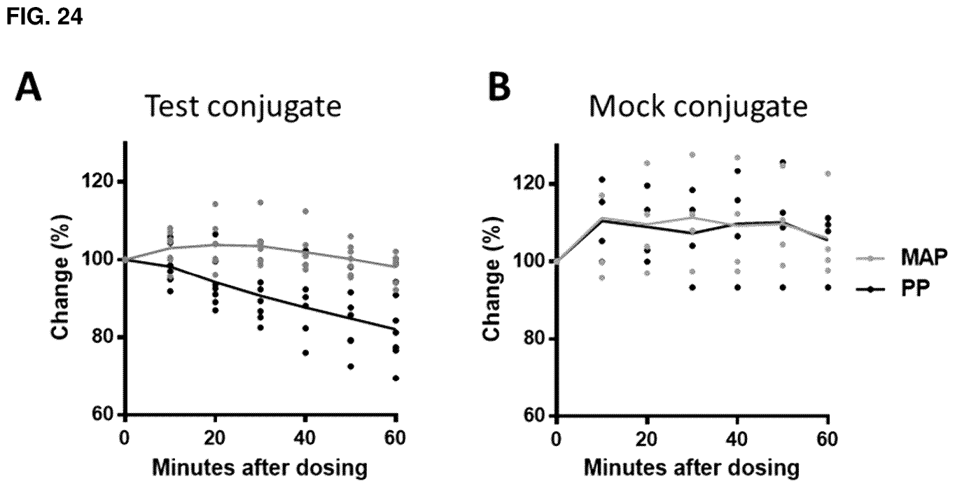

In BDL rats, infusion of the test conjugate, but not of the mock conjugate, resulted in a gradual decrease of portal pressure (PP) while arterial pressure remained constant (MAP), indicating selective relief of portal hypertension (FIG. 24A). In animals infused with the mock conjugate, both pressures remained constant, indicating that the lowering of portal pressure by the test conjugate was mediated by the small molecule kinase inhibitor (FIG. 24B). In SHAM rats, neither the test conjugate nor the mock conjugate affected portal or arterial pressure, indicating that the effect observed in BDL rats was specific to the diseased condition.

### 15.2. In Vivo Targeting of the IGF2R by an IGF2R-Binding Molecule of the Invention Conjugated to a Small Molecule Kinase Inhibitor

After isolation, livers obtained from test conjugate-infused BDL and SHAM rats were fixated with paraformaldehyde, soaked in 30% (w/v) sucrose in PBS, and sectioned using cryo-microtome.

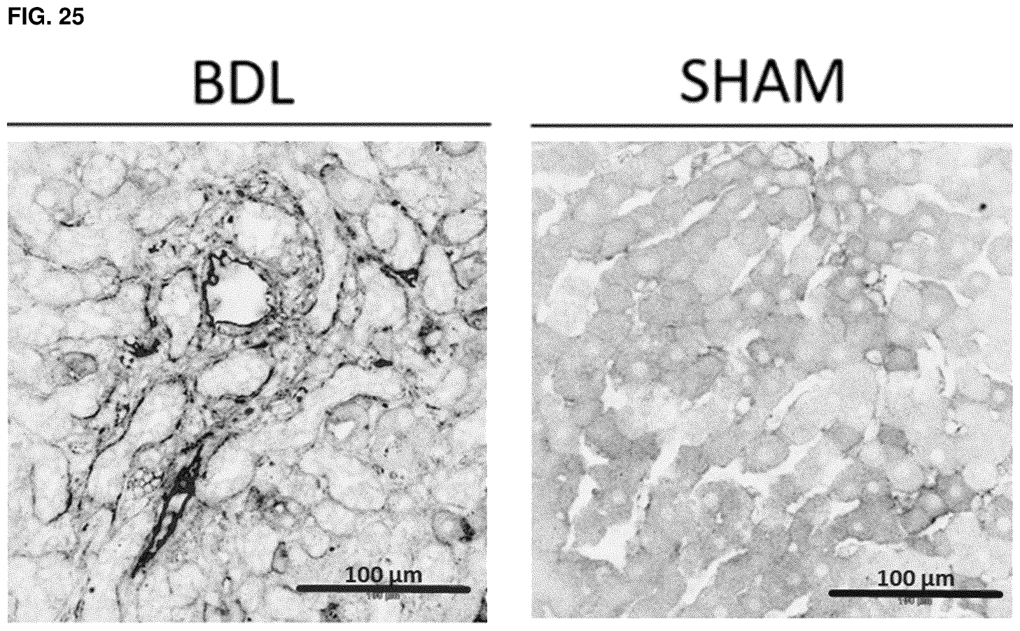

To detect the test conjugate in situ, tissue sections were stained with a rabbit polyclonal anti-VHH antiserum recognizing the 13F11-13E8 moiety of the conjugate, followed by an anti-rabbit antibody conjugated to the fluorophore Alexa-488 for visualization by confocal fluorescence microscopy (cf. example 14). As shown in FIG. 25, the test conjugate was detected in the liver of BDL rats and not in the liver of SHAM rats. Furthermore, a second staining of the same sections with excess 13F11-13E8 conjugated to the fluorophore Alexa-647 (13F11-13E8-A647, conjugation method as described in example 10.2.) demonstrated that the distribution of the test conjugate in the liver of BDL rats overlapped with the (tissue) distribution of IGF2R (not shown). These results indicate that the presence of test conjugate in the liver was driven by the (cirrhosis-specific) expression of IGF2R, and in vivo targeting of the molecular target had in fact been achieved.

1.-48. (canceled)

49. A binding molecule comprising
at least one diagnostic or therapeutic moiety, and
at least two single variable domains of a heavy-chain-only antibody (VHH), each one able to specifically bind to a platelet-derived growth factor receptor beta (PDGFRB) that is expressed on a fibrogenic effector cell identifiable by the expression of alpha smooth muscle actin and/or stress fibers discernable by immune fluorescence microscopy,
wherein a first one of the at least two single variable domains comprises a CDR 1 sequence according SEQ ID NO: 11, a CDR 2 sequence according to SEQ ID NO: 13 and a CDR 3 sequence according to SEQ ID NO: 15;
and
wherein a second one of the at least two single variable domains comprises:
a CDR 1 sequence according SEQ ID NO: 67, a CDR 2 sequence according to SEQ ID NO: 69 and a CDR 3 sequence according to SEQ ID NO: 71, or
a CDR 1 sequence according SEQ ID NO: 83, a CDR 2 sequence according to SEQ ID NO: 85 and a CDR 3 sequence according to SEQ ID NO: 87, or
a CDR 1 sequence according SEQ ID NO: 155, a CDR 2 sequence according to SEQ ID NO: 157 and a CDR 3 sequence according to SEQ ID NO: 159;
or
wherein a first one of the at least two single variable domains comprises at least one single variable antibody domain comprising a CDR 1 sequence according to SEQ ID NO: 67, a CDR 2 sequence according to SEQ ID NO: 69 and a CDR 3 sequence according to SEQ ID NO: 71;
and
wherein a second one of the at least two single variable domains comprises a CDR 1 sequence according SEQ ID NO: 83, a CDR 2 sequence according to SEQ ID NO: 85 and a CDR 3 sequence according to SEQ ID NO: 87;
wherein any one of the first one and/or of the second one of the at least two single variable domains comprises at most 4 amino acids that have been conservatively substituted per any one of the CDR sequences, independently.

50. The binding molecule according to claim 49, wherein the first one of the at least two single variable domains comprises a CDR 1 sequence according SEQ ID NO: 11, a CDR 2 sequence according to SEQ ID NO: 13 and a CDR 3 sequence according to SEQ ID NO: 15;
and wherein the second one of the at least two single variable domains comprises a CDR 1 sequence according SEQ ID NO: 155, a CDR 2 sequence according to SEQ ID NO: 157 and a CDR 3 sequence according to SEQ ID NO: 159,
wherein any one of the first one and/or of the second one of the at least two single variable domains comprises at most 4 amino acids that have been conservatively substituted per any one of the CDR sequences, independently.

51. The binding molecule according to claim 50, wherein the first one of the at least two single variable domains comprises or consists of a VHH sequence according to SEQ ID NOs: 9 (SP02P), and wherein the second one of the at least two single variable domains comprises or consists of a VHH sequence according to any one of SEQ ID NOs: 153 (SP26P).

52. The binding molecule according to claim 49, wherein any one of the first one and/or of the second one of the at least two single variable domains comprises at most 3 amino acids that have been conservatively substituted per any one of the CDR sequences, independently.

53. The binding molecule according to claim 49, wherein any one of the first one and/or of the second one of the at least two single variable domains comprises at most 2 amino acids that have been conservatively substituted per any one of the CDR sequences, independently.

54. The binding molecule according to claim 49, wherein any one of the first one and/or of the second one of the at least two single variable domains comprises at most 1 amino acids that has been conservatively substituted per any one of the CDR sequences, independently.

55. The binding molecule according to claim 49, wherein the binding molecule is biparatopic.

56. The binding molecule according to claim 49, wherein the at least two single variable domains are separated by a linker amino acid sequence.

57. The binding molecule according to claim 49, wherein the binding molecule comprises an N-terminal or a C-terminal cysteine residue.

58. The binding molecule according to claim 1, wherein the binding molecule comprises a half-life extender that provide the binding molecule with increased half-life compared to a binding molecule without a half-life extender.

59. The binding molecule or the method according to claim 58, wherein the half-life extender comprises an albumin binding domain or an albumin binding VHH or a fragment thereof.

60. The binding molecule according to claim 49, wherein the therapeutic molecule is able to inhibit any one or more of contractile activity, fibrogenic activity, chemotactic activity and pro-inflammatory activity of the fibrogenic effector cell.

61. The binding molecule according to claim 49, wherein the therapeutic moiety is a kinase inhibitor.

62. The binding molecule according to claim 61, wherein the kinase inhibitor selected from Rho-kinase inhibitors, JAK-2 inhibitors or neprilysin inhibitors, or Angiotensin II Receptor antagonists, such as losartan, preferably wherein the therapeutic moiety is a Rho-kinase inhibitor or a JAK-2 inhibitor.

63. The binding molecule according to claim 49 comprising a diagnostic moiety.

64. The binding molecule according to claim 63, wherein the diagnostic moiety is an imaging agent.
